(19) United States
(12) Patent Application Publication
(10) Pub. No.: US 2008/0261206 A1
(43) Pub. Date: Oct. 23, 2008

(54) Oligonucleotide for Detection of a Microorganism, Diagnostic Kits and Methods for Detection of Microorganisms Using the Oligonucleotide

(75) Inventors: Cheol-Min Kim, Busan-city (KR); Hee-Kyung Park, Seoul (KR); Eun-Sil Song, Busan-city (KR); Jun-Hyung Park, Busan-city (KR); Hyun-Jung Jang, Busan-city (KR); Byeong-Chul Kang, Busan-city (KR)

Correspondence Address: THE RAFFERTY PATENT LAW FIRM, 5641 BURKE CENTRE PKWY, SUITE 100, BURKE, VA, 22015-2259, US

(73) Assignee: GENEIN CO., LTD., Busan (KR)

(21) Appl. No.: 11/574,405

(22) PCT Filed: Aug. 28, 2005
(86) PCT No.: PCT/KR05/02825

§ 371 Date: Feb. 27, 2007

(30) Foreign Application Priority Data
Aug. 28, 2004 (KR) 10-2004-0068313

(51) Int. Cl. C12Q 1/68; C40B 40/06; C07H 21/00

(52) U.S. Cl. 435/6; 506/16; 536/24.32

(57) ABSTRACT

The present invention relates to a method so called Bacterial Digitalcode System (BaDis) that identifies microorganism by using bacterial-specific, genus-specific and species-specific oligonucleotides from a variety of samples or specimens for detection and differential diagnosis of microorganism. Particularly, the present invention relates to bacterial-specific, genus-specific and species-specific oligonucleotides designed by the target nucleotide sequences of 23S rDNA or ITS gene, polymerase chain reaction (hereinafter, referred to as “PCR”) kits using the oligonucleotides as a primer, the microarray containing the oligonucleotides as a probe, and methods for detecting microorganism by using the oligonucleotides. Therefore, the present invention can be applied to detect the presence of microorganism and diagnose differentially all microorganism such as pathogenic bacteria of infectious diseases, bacteria inducing food poisoning, bacteria contaminating biomedical products and environmental pollutants.

### TECHNICAL FIELD

The present invention relates to oligonucleotides useful for detection (herein, also referred to as differential diagnosis) of microorganisms (herein, also referred to as bacteria) and methods for detecting microorganisms by using the same, more particularly to bacterial-specific, genus-specific and species-specific oligonucleotides designed from the target nucleotide sequences of 23S rDNA gene or ITS for the differential diagnosis, diagnostic kits using the oligonucleotides as primers or probes, and methods for detecting microorganisms by using the oligonucleotides.

### BACKGROUND ART

Conventional cell culture methods and biochemical methods for identifying bacteria require a long time period, difficult analytic procedures and complicated manipulations (J. Clin. Microbiology, 12: 3674˜3679, 1998). In the last decade, the methods for detecting microorganisms have advanced to exploit antibodies and fluorescence, enzyme-linked immunosorbent assay (ELISA) and the like. However, there are several disadvantages. They fail to catch minor microorganisms, spend a great deal of cost and time, and need trained workers. Accordingly, it is necessary to develop a rapid and reliable process. Recently, several nucleic acid amplifications based upon the molecular biological method are spotlighted to have the sensitivity and specificity by polymerase chain reactions (PCR) and DNA chips. The PCR method is so efficient to amplify a particular domain of gene exponentially by using very small amount of DNA. It is applied widely to detect minor microorganisms through a molecular biological technique because of a high diagnostic capacity. The DNA chips are a technique based upon the hybridization principle of probes. It is reported to analyze a lot of genes onto a solid substrate simultaneously, because tens or ten thousands kinds of genetic material are attached densely in a very small amount. Also, it is advantageous to identify a genotype, isolate a mutant, and analyze the gene expression and the like. Especially, the identification of genotype in such a biotechnological diagnosis is a highly advanced technique to detect any microbe of clinical specimen at a time rapidly and sensitively, even though the microbe grows slowly, is cultured hardly or not described yet.

Referring to several literatures, gene probes are designed on a basis of 16S rDNA containing a conservative sequence in overall microorganisms and utilized in order to identify a pathogenic microbe of infectious disease (J. Microbiol. Methods, 55: 541˜555, 2003; Pediatrics, 95: 165˜169, 1995; Appl. Environ. Microbiol., 64: 795˜799, 1998; J. Clin. Microbiol., 32: 335˜351, 1994; Microbiol., 148: 257˜266, 2002). However, this gene is disadvantageous to diagnose particular microorganism due to lacking in small variable region. Recently, several new probes are designed to detect microorganisms on basis of ITS (internal transcribed spacer region) containing a hyper-variable region and 23S rDNA not fully determined in the nucleotide sequence yet (J. Clin. Microbiol., 38: 4080˜4085, 2000; Microbiol., 142: 3˜16, 1996; GENE, 238: 241˜252, 1999; FEMS Microbiol. Letters, 187: 167˜173, 2000; J. Clin. Microbiol., 38: 781˜788, 2000; J. Microbiol. Methods, 53: 245˜252, 2003). However, these genes may not discriminate several different bacteria or several species of pathogens belonging to the same genus presently. In practice, it is necessary to detect all the bacteria together, because several microorganisms belonging to different genera contaminate a biological medicine produced from a cell tissue or whole blood. The DNA chips enable overall microorganisms to be diagnosed at a time.

To overcome the foregoing limitation in traditional methods, a novel diagnostic method should be developed to identify unknown microorganisms in a clinical specimen or in a natural specimen separated from environment and to screen several kinds of microorganisms simultaneously. In order to settle above-mentioned problems, the present inventors have tried to manufacture novel primers or probes which exploit 23S rDNA gene useful to design bacterial-specific and bacterial genus-specific primers or their probes and ITS useful to design bacterial species and subspecies-specific primers or their probes and completed the invention successfully.

### DISCLOSURE OF INVENTION

The main object of the present invention is to provide bacterial-specific oligonucleotides derived from 23S rDNA gene to examine the presence of general microorganism by the primary screening; bacterial genus-specific oligonucleotides derived from 23S rDNA gene by the secondary screening; and bacterial species or subspecies-specific oligonucleotide derived from ITS by the tertiary screening for a microbial diagnosis.

In addition, another object of the present invention is to provide a diagnostic PCR kit and a microarray comprising the oligonucleotides of the present invention as a primer and a probe for a microbial diagnosis.

In addition, another object of the present invention is to provide a method for detecting and diagnosing microorganism by using the diagnostic PCR kit and the microarray of the present invention. The method for detecting microorganism can omit a complicated manipulation, reduce a diagnostic cost and detect even hardly cultured microorganisms for diagnosis. Further, the method for detecting microorganism can identify a pathogenic microbe exactly and prevent the abuse of antibiotics caused by delayed diagnosis and mis-diagnosis.

Bacterial Digitalcode System (BaDis) is referred to an identification and differential diagnosis system for microorganism, comprising all or a part of primers or probes specific for general bacteria, bacterial genus, bacterial species and subspecies.

In order to achieve the object of the present invention, the present invention provides a bacterial-specific oligonucleotide, which contains one or more sequences selected among SEQ ID NO: 1 to 19 or their complementary sequences and enables a diagnosis of bacteria. Any oligonucleotide selected above can be used to primarily detect the presence of bacteria, since it amplifies and hybridizes the 23S rDNA gene of all bacteria.

In order to achieve another object, the present invention provides a bacterial genus-specific oligonucleotide, which contains one or more sequences selected among SEQ ID NO: 20 to 189 or their complementary sequences and enables a differential diagnosis of a specific bacterial genus. Any oligonucleotide selected above can be used to detect and identify a specific genus to which a pathogenic microbe belongs, since it amplifies and hybridizes 23S rDNA gene of different genuses specifically.

Particularly, the oligonucleotides of SEQ ID NO: 20 to 22 can detect and identify genus Acinetobacter specifically; the oligonucleotides of SEQ ID NO: 23 to 28, genus Aeromonas; the oligonucleotides of SEQ ID NO: 29 to 34, genus Bacillus; the oligonucleotides of SEQ ID NO: 35 to 41, genus Bacteroides; the oligonucleotides of SEQ ID NO: 42 to 44, genus Bordetella; the oligonucleotides of SEQ ID NO: 45 to 47, genus Borrelia; the oligonucleotides of SEQ ID NO: 48 to 50, genus Brucella; the oligonucleotides of SEQ ID NO: 51 to 53, genus Burkholderia; the oligonucleotides of SEQ ID NO: 54 to 56, genus Campylobacter, the oligonucleotides of SEQ ID NO: 57 to 59, genus Chlamydia; the oligonucleotides of SEQ ID NO: 60 to 65, genus Citrobacter, the oligonucleotides of SEQ ID NO: 66 to 71, genus Clostridium; the oligonucleotides of SEQ ID NO: 72 to 74, genus Corynebacterium; the oligonucleotides of SEQ ID NO: 75, genus Enterbacter, the oligonucleotides of SEQ ID NO: 76 to 80, genus Enterococcus; the oligonucleotides of SEQ ID NO: 81 to 86, genus Fusobacterium; the oligonucleotides of SEQ ID NO: 87 to 89, genus Haemophilus; the oligonucleotides of SEQ ID NO: 90 to 96, genus Helicobacter, the oligonucleotides of SEQ ID NO: 97 to 102, genus Klebsiella; the oligonucleotides of SEQ ID NO: 103 to 108, genus Legionella; the oligonucleotides of SEQ ID NO: 109 to 114, genus Listeria; the oligonucleotides of SEQ ID NO: 115 to 117, genus Morganella; the oligonucleotides of SEQ ID NO: 118 to 123, genus Mycobacteria; the oligonucleotides of SEQ ID NO: 124 to 129, genus Mycoplasma; the oligonucleotides of SEQ ID NO: 130 to 135, genus Neisseria; the oligonucleotides of SEQ ID NO: 136 to 138, genus Peptococcus; the oligonucleotides of SEQ ID NO: 139 to 141, genus Plesiomonas; the oligonucleotides of SEQ ID NO: 142 to 144, genus Porphyromonas; the oligonucleotides of SEQ ID NO: 145 to 147, genus Propionibacterium; the oligonucleotides of SEQ ID NO: 148 to 151, genus Providencia; the oligonucleotides of SEQ ID NO: 152 to 157, genus Pseudomonas; the oligonucleotides of SEQ ID NO: 158 to 160, genus Salmonella; the oligonucleotides of SEQ ID NO: 161 to 164, genus Shigella; the oligonucleotides of SEQ ID NO: 165 to 170, genus Staphylococcus; the oligonucleotides of SEQ ID NO: 171 to 176, genus Streptococcus; the oligonucleotides of SEQ ID NO: 177 to 179, genus Treponema; the oligonucleotides of SEQ ID NO: 180 to 182, genus Ureaplasma; the oligonucleotides of SEQ ID NO: 183 to 185, genus Vibrio; and the oligonucleotides of SEQ ID NO: 186 to 189, genus Yersinia.

In order to design novel oligonucleotides for a differential diagnosis of microorganism, the present inventors have analyzed the nucleotide sequences of 23S rDNA genes of various microorganisms not disclosed yet. As a result, we have newly determined 37 different kinds of the nucleotide sequences (temporary SEQ NO: 1 to 37; not shown) from the 23S rDNA genes. The oligonucleotides of the present invention are designed on a basis of the multiple alignment and the BLAST analysis in 23S rDNA genes that are derived from various bacteria and include 37 kinds of the nucleotide sequences newly disclosed above. The oligonucleotides can be applied as an amplifiable primer for specific nucleotide sequences in order to detect the presence of microorganism and to enable a bacterial genus-specific diagnosis of pathogens.

In order to achieve another object, the present invention provides a set of amplifiable primers comprising one or more selected among the bacterial-specific and bacterial genus-specific oligonucleotides to enable a differential diagnosis. The set of primers can be used to manufacture the PCR kits of the present invention.

In order to achieve another object, the present invention provides a set of diagnostic probes comprising one or more selected among the bacterial-specific and bacterial genus-specific oligonucleotides to enable a differential diagnosis. The set of probes can be used to manufacture the microarray of the present invention.

In order to achieve another object, the present invention provides a diagnostic kit comprising one or more selected among the bacterial-specific and bacterial genus-specific oligonucleotides to enable a differential diagnosis.

In the diagnostic kit of the present invention, the oligonucleotides can be labeled with radioactive or non-radioactive substance. Preferably, the non-radioactive substance can be selected among biotin, digoxigenin (Dig), FRET (fluorescence resonance energy transfer), fluorescent label such as Cy5, Cy3 and the like. The oligonucleotides can be used as a primer or probe and further, other primers can be added to amplify a target DNA.

In order to achieve another object, the present invention provides a diagnostic PCR kit comprising one set of primers containing the bacterial-specific oligonucleotides and the bacterial genus-specific oligonucleotides for a differential diagnosis.

Preferably, the PCR kit of the present invention is further comprised of bacterial species-specific oligonucleotides as a primer for the differential diagnosis. The bacterial species-specific oligonucleotides can be any oligonucleotide selected from species-specific primers of pathogenic microbes conventionally known in this arts. Preferably, the bacterial species-specific oligonucleotides can be the nucleotide sequence (TGCATGACAACAAAG) specific for Mycobacterium tuberculosis; the nucleotide sequence (GTAAATTAAACCCAAATCCC) specific for Mycoplasma pneumoniae; and the like.

Preferably, the PCR kit of the present invention is further comprised of DNA polymerase, 4 dNTPs (ATP, GTP, CTP, TTP) mixture, PCR buffer solutions, a user's manual and the like. The target nucleotide sequences can be polymerized by performing a Taq DNA polymerase-based amplification, Klenow fragment-based amplification, Phi29 polymerase-based amplification, Helicase-dependent amplification or the like, depending upon the kinds of DNA polymerase.

In order to achieve another object, the present invention provides a microarray comprising the bacterial-specific oligonucleotides and the bacterial genus-specific oligonucleotides attached onto a substrate as a probe.

Preferably, the microarray of the present invention is further comprised of bacterial species-specific oligonucleotides as a primer for a differential diagnosis. The bacterial species-specific oligonucleotides can be any oligonucleotide selected from species-specific primers of pathogenic microbes conventionally known in this arts. Preferably, the bacterial species-specific oligonucleotides can be the nucleotide sequence (TGCATGACAACAAAG) specific for Mycobacterium tuberculosis; the nucleotide sequence (GTAAATTAAACCCAAATCCC) specific for Mycoplasma pneumoniae; and the like.

In the microarray of the present invention, the probe can be a general nucleic acid such as deoxynucleotide (DNA) and ribonucleotide (RNA) and further, a nucleic acid derivative selected among peptide nucleotide (PNA), locked nucleotide (LNA) and dihexynucleotide (HNA). Advantageously, the nucleic acid derivative is resistant to enzymes such as nuclease, has the high specificity for nucleotide sequences structurally and is thermo-resistant.

In the PCR kit and the microarray of the present invention, the primer and probe can be manufactured to have a sense or anti-sense sequence. Preferably, the oligonucleotides of the present invention can contain one or more sequences selected among the above nucleotide sequences of SEQ ID NOS or their complementary sequences.

Preferably, the substrate in the microarray of the present can be made of slide glass, plastic, membrane, semi-conductive chip, silicon, gel, nano material, ceramic, metallic substance, optical fiber or their mixture. Preferably, the microarray of the present can be manufactured by a pin microarray (Microarray printing technology, Don Rose, Ph.D., Cartesian Technologies, Inc., Anal. Biochem., 320(2): 281˜91, 2003); ink jet (Nat. Biotech., 18: 438˜441, 2000; Bioconjug. Chem., 13(1): 97˜103, 2002); photolithography (Cur. Opinion Chem. Biol., 2: 404˜410, 1998; Nature genetics supplement, 21: 20˜24, 1999); or electric array (Ann. Biomed. Eng., 20(4): 423˜37, 1992; Psychiatric Genetics, 12: 181˜192, 2002) techniques conventional in this arts.

Preferably, the microarray of the present invention is further comprised of hybridization reagents, a PCR kit containing primers for the amplification of target genes, a washing buffer for removing non-hybridized DNAs, a cover slip, a staining solution, a washing buffer for removing free dye, a user's manual and the like, if provided with a diagnostic kit.

In order to achieve another object, the present invention provides a diagnostic method for detecting and identifying microorganism, comprising steps as follows: (1) purifying nucleic acids from a specimen; (2) amplifying a target DNA within the nucleic acids by using the diagnostic PCR kit; and (3) analyzing the amplified DNA by performing a gel electrophoresis.

In the diagnostic method for detecting and identifying microorganism, the step (2) amplifying a target DNA within the nucleic acids can be accomplished by a modified PCR procedure selected among Hot-start PCR, Nested PCR, Multiplex PCR, reverse transcriptase PCR (RT-PCR), degenerate oligonucleotide primer PCR (DOP PCR), Quantitative RT-PCR, In-Situ PCR, Micro PCR, or Lab-on a chip PCR, as well as by general PCR procedures.

Advantageously, the modified procedures have a still higher efficiency to detect microorganism. In detail, the RT-PCR can detect transcribed DNAs indicating an activated infection; the In-Situ PCR detects bacteria within a tissue; the Micro PCR amplifies a very small amount of DNA or RNA in a tube or capillary; the Lab-on a chip PCR performs several steps at a time, from DNA extraction, PCR, gel electrophoresis, to DNA quantitation; and the like.

In order to achieve another object, the present invention provides a diagnostic method for detecting and identifying microorganism, comprising steps as follows: (1) purifying nucleic acids from a specimen; (2) amplifying a tyramide signal or other signals using a gold nano-particle probe and Raman-active dye, after or without the step amplifying a target DNA within nucleic acids; and (3) detecting a fluorescent signal from the DNA and RNA amplified above.

In the diagnostic method of the present invention, the tyramide signal amplification (Nucleic Acids Res., 30:e4, 2002) or the signal amplification using a gold nano-particle probe and Raman-active dye (Science, 297: 1536˜1540, 2002) can be accomplished after or without the step amplifying a target DNA within nucleic acids. In detail, first the tyramide signal amplification is comprised of following steps: (1) cultivating a tissue or cell specimen; (2) extracting DNA or RNA from the specimen; (3) performing a PCR amplification; (4) hybridizing onto a microarray; and (5) screening a fluorescent signal. Second, the signal amplification using a gold nano-particle probe and Raman-active dye is comprised of following steps: (1) extracting DNA or RNA from a specimen; (2) performing a PCR amplification; (3) hybridizing onto a microarray attaching modified gold nano-particles with Raman-active fluorescence, Cy3 group; and (5) screening a fluorescent signal in a Raman spectrum.

In order to achieve another object, the present invention provides a diagnostic method for detecting and identifying microorganism, comprising steps as follows: (1) purifying nucleic acids from a specimen; (2) amplifying a target DNA within the nucleic acids; (3) hybridizing the amplified DNA with the probes onto the microarray of the present invention; and (4) detecting a signal generated from the DNA hybrid.

In the diagnostic method of the present invention for detecting and identifying microorganism, the specimen can be blood, body fluid, tissue, sputum, feces, urine, pus or the like. The nucleic acids can be separated by performing a conventional process purifying DNA or RNA or by using a purification kit. The target DNA can be amplified by performing a conventional PCR. The microorganism can be detected by performing a conventional agarose gel electrophoresis. The hybrid signal can be detected with a commercially available scanner after binding a conventional fluorescent dye such as Cy5 or Cy3.

Preferably, the present invention provides the method for detecting and identifying microorganism, wherein one or more bacteria selected from a group comprising genus Acinetobacter (SEQ ID NO: 20 to 22), genus Aeromonas (SEQ ID NO: 23 to 28), genus Bacillus (SEQ ID NO: 29 to 34), genus Bacteroides (SEQ ID NO: 35 to 41), genus Bordetella (SEQ ID NO: 42 to 44), genus Borrelia (SEQ ID NO: 45 to 47), genus Brucella (SEQ ID NO: 48 to 50), genus Burkholderia (SEQ ID NO: 51 to 53), genus Campylobacter (SEQ ID NO: 54 to 56), genus Chlamydia (SEQ ID NO: 57 to 59), genus Citrobacter (SEQ ID NO: 60 to 65), genus Clostridium (SEQ ID NO: 66 to 71), genus Corynebacterium (SEQ ID NO: 72 to 74), genus Enterbacter (SEQ ID NO: 75), genus Enterococcus (SEQ ID NO: 76 to 80), genus Fusobacterium (SEQ ID NO: 81 to 86), genus Haemophilus (SEQ ID NO: 87 to 89), genus Helicobacter (SEQ ID NO: 90 to 96), genus Klebsiella (SEQ ID NO: 97 to 102), genus Legionella (SEQ ID NO: 103 to 108), genus Listeria (SEQ ID NO: 109 to 114), genus Morganella (SEQ ID NO: 115 to 117), genus Mycobacteria (SEQ ID NO: 118 to 123), genus Mycoplasma (SEQ ID NO: 124 to 129), genus Neisseria (SEQ ID NO: 130 to 135), genus Peptococcus (SEQ ID NO: 136 to 138), genus Plesiomonas (SEQ ID NO: 139 to 141), genus Porphyromonas (SEQ ID NO: 142 to 144), genus Propionibacterium (SEQ ID NO: 145 to 147), genus Providencia (SEQ ID NO: 148 to 151), genus Pseudomonas (SEQ ID NO: 152 to 157), genus Salmonella (SEQ ID NO: 158 to 160), genus Shigella (SEQ ID NO: 161 to 164), genus Staphylococcus (SEQ ID NO: 165 to 170), genus Streptococcus (SEQ ID NO: 171 to 176), genus Treponema (SEQ ID NO: 177 to 179), genus Ureaplasma (SEQ ID NO: 180 to 182), genus Vibrio (SEQ ID NO: 183 to 185), and genus Yersinia (SEQ ID NO: 186 to 189), can be diagnosed simultaneously. Accordingly in the present invention, the diagnostic method for detecting several kinds of bacteria from a specimen is provided.

In order to achieve another object, the present invention provides a diagnostic method for detecting and identifying microorganism, wherein SBE (Single base extension), Sequencing, RFLP (Restriction fragment length polymorphism), REA (Restriction endonuclease analysis) or the like are accomplished on a basis of the difference of one nucleotide by using bacterial-specific oligonucleotides designed to detect the presence of bacteria; and bacterial genus-specific oligonucleotides and bacterial species-specific and subspecies-specific oligonucleotides designed to enable the differential diagnosis.

Hereinafter, the present invention will be described more clearly as follows.

The present invention relates to a method for detecting the presence of microorganism and identifying a bacterial genus of pathogens exactly, which is a sort of genetic test using an oligonucleotide for diagnosing bacteria. The method for detecting the presence of microorganism and identifying a bacterial genus of pathogens is comprised of several steps as follows.

First, the PCR process is comprised of steps:
(1) purifying nucleic acids from a cultured or clinical specimen, if necessary;
(2) amplifying whole or a part of the target DNA sequence by using one or more pairs of proper primers, if necessary
(3) performing a gel electrophoresis.

Second, the microarray process is comprised of steps:
(1) purifying nucleic acids from a cultured or clinical specimen, if necessary;
(2) amplifying whole or a part of the target DNA sequence by using one or more pairs of proper primers, if necessary;
(3) hybridizing the nucleic acids obtained in step (1) and/or step (2) with a bacterial-specific, bacterial genus-specific or bacterial species-specific oligonucleotide acting as a probe sequence, reverse probe sequence, or their complementary sequence of probe;
(4) detecting a hybrid reacted in step (3)
(5) diagnosing an plausible infection of microorganism by analyzing a hybrid signal resulted from step (4).

The present inventors have determined the nucleotide sequences of 23S rDNA genes and ITS in order to design oligonucleotides detecting the presence of microorganism and enabling the differential diagnosis for a bacterial genus and species. As a consequence, we have obtained bacterial-specific, genus-specific and species-specific sequences and thus, developed a highly specific and sensitive PCR method and a hybridization method to detect the presence of microorganism and identify a bacterial genus and species. Further, we have found and newly analyzed 37 different kinds of the nucleotide sequences from the 23S rDNA genes of microorganism, which permits more specific and sensitive primers and probes to be designed and thus, enables a bacterial genus and species to be identified exactly.

### BRIEF DESCRIPTION OF THE DRAWINGS

The above and other objects, features and other advantages of the present invention will be more clearly understood from the following detailed description taken in conjunction with the accompanying drawings, in which;

FIG. 1 depicts the overall flowchart of the present invention;

FIG. 2 depicts the target region and the position of primers and probes adopted to amplify a microbial gene from a biological specimen;

FIG. 3 depicts the partial data of multiple alignment of conservative nucleotide sequences in the 23S rDNA gene of each bacterial genus to design a bacterial-specific primer;

FIG. 4 depicts the result of PCR amplification with a pair of primers designed by using a bacterial-specific nucleotide sequence;

FIG. 5a depicts the multiple alignment of conservative nucleotide sequences in the 23S rDNA gene of each Mycobacteria sp. to design Mycobacteria specific primer;

FIG. 5b depicts the multiple alignment of conservative nucleotide sequences in the 23S rDNA gene of each Staphylococcus sp. to design Staphylococcus-specific primer;

FIG. 6a˜6d depict the results of PCR amplification by using a pair of primers designed by a bacterial genus-specific nucleotide sequence, respectively in Aeromonas, Enterococcus, Mycobacteria and Streptococcus;

FIG. 7a depicts the microarray comprising a substrate with one set of probes to detect the presence of microorganism;

FIG. 7b˜6c depict the result of hybridization by using each specific probe after performing the image analysis and estimating the intensity of its image elements;

FIG. 8a depicts the microarray comprising a substrate with one set of probes to detect the presence of microorganism and identify a bacterial genus of pathogens;

FIG. 8b depicts the result of hybridization by using specific probes of Streptococcus sp. after performing the image analysis and estimating the intensity of its image elements;

FIG. 9a depicts the microarray comprising a substrate with one set of probes to detect the presence of microorganism and identify a bacterial genus and species of pathogens together;

FIG. 9b depicts the result of hybridization by using specific probes of genus Mycobacteria and Mycobacterium tuberculosis, after performing the image analysis and estimating the intensity of its image elements;

FIG. 9c depicts the result of hybridization by using specific probes of genus Mycoplasma and Mycoplasma pneumoniae, after performing the image analyses and estimating the intensities of their image elements.

### BEST MODE FOR CARRYING OUT THE INVENTION

Hereinafter, the present invention will be described more clearly with attached drawings as follows.

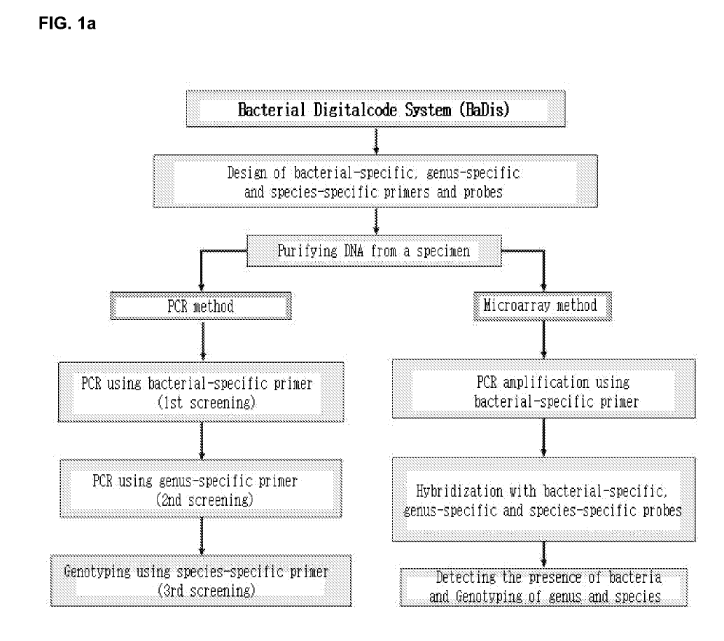

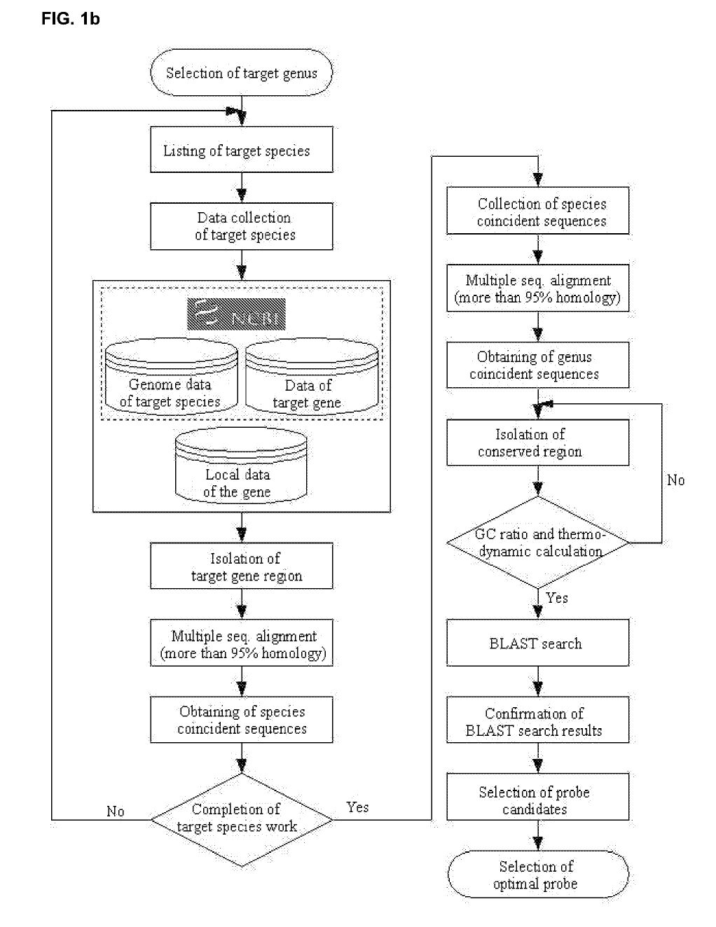

FIG. 1 depicts the overall flowchart of the present invention. FIG. 1a illustrates the flowchart that designs bacterial-specific, genus-specific and species-specific primers and probes by using a microbial identification system so called Bacterial Digitalcode System (BaDis); extracts DNAs from a cultured and clinical specimen; detects the presence of microorganism by the gene amplification such as PCR method and the microarray method; and further, identifies the genotype of microbial genus and species orderly or at a time.

FIG. 1b illustrates the flowchart that accomplishes the multiple alignment of target regions collected from NCBI and our data retained. The multiple alignment is conducted by using Clustal W. The homology is set up at more than 95% of critical value to judge the identical sequence. The resulting sequence is used to separate a conservative region identifying general microorganism or a microbial genus. Then, the conservative sequence region is examined to estimate GC ratio considering thermodynamic problems, and judged by the BLAST analysis whether it detects general microorganism or identifies a microbial genus or not. Finally, the candidate group of probes can be selected.

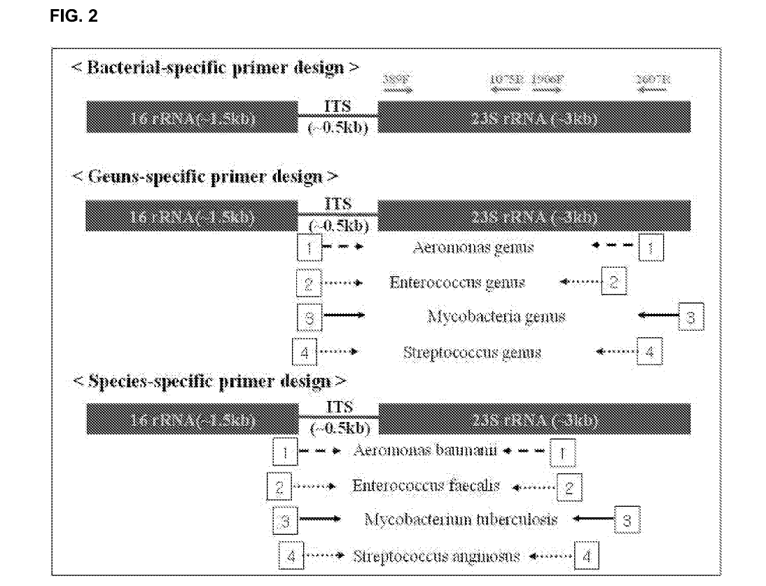

FIG. 2 depicts the target region and the position of primers and probes adopted to amplify a microbial gene from a biological specimen. The general bacterial-specific and the bacterial species-specific primers and probes are designed by using common 16S rDNA gene of almost all bacteria and 23S rDNA gene not fully disclosed yet. In order to identify rare bacteria not discriminated even by using the 23S rDNA gene, the bacterial genus and species-specific nucleotide sequences are designed by combining ITS.

The primers and probes of the present invention for detecting the presence of microorganism and identifying a bacterial species are designed on a basis of the multiple alignment of 23S rDNA nucleotide sequences. The multiple alignment is conducted by using available Clustal W. The identical sequence is separated, if reaching more than 95% of homology in the multiple sequence data. The sequence region having less than 95% is denoted to “N” to isolate the identical sequence entirely.

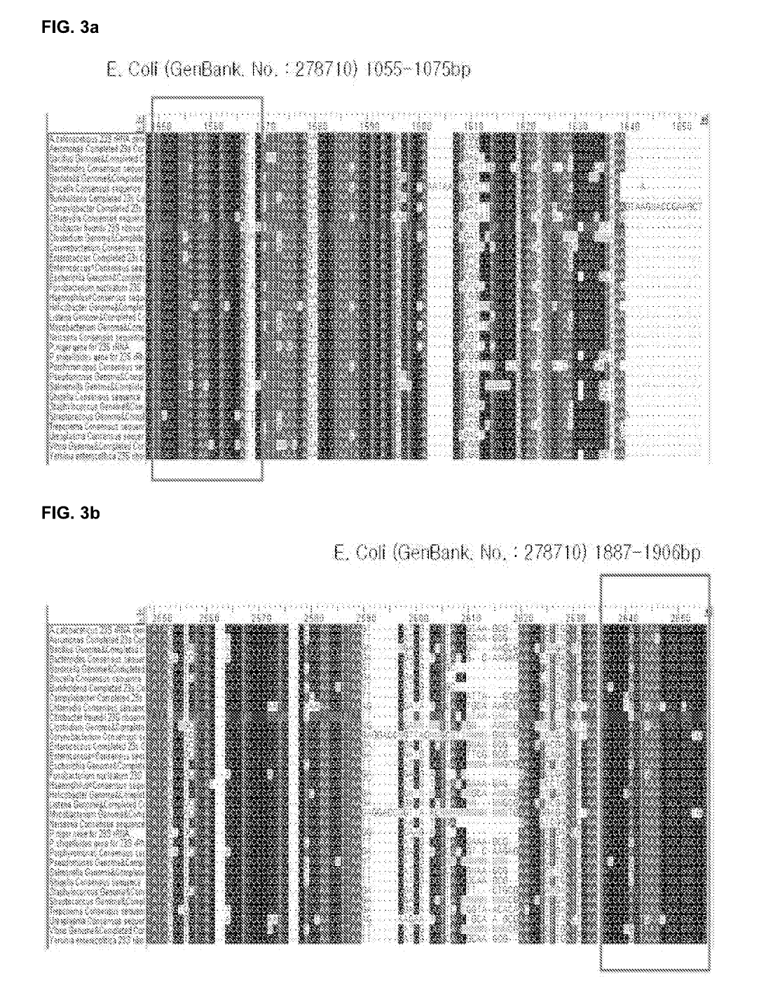

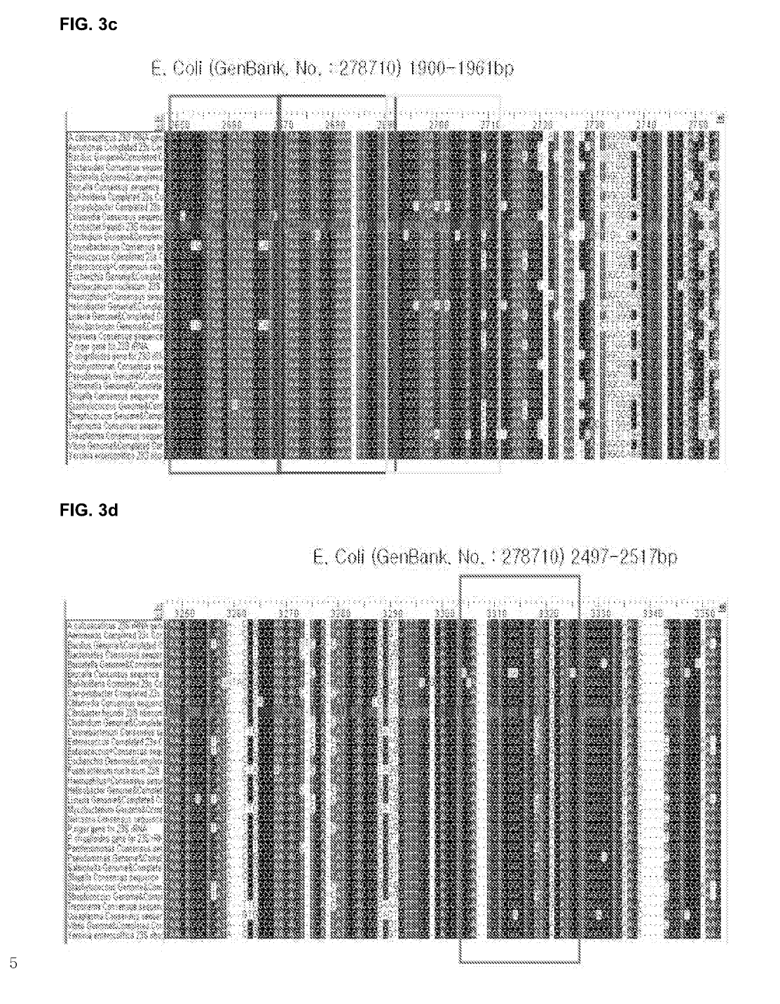

FIG. 3 depicts the multiple alignment of conservative nucleotide sequences in the 23S rDNA gene to design a specific primer detecting the presence of microorganism. The bacterial-specific oligonucleotide is designed by using a conservative sequence found in all microorganisms (in box).

In a preferred embodiment of the PCR method of the present invention, the target sequence of microorganism is amplified in Step (2) by using one or more pairs of proper primers to detect the presence of microorganism. The PCR is performed in a standard strain by using the primers for the amplification described in Example 1.

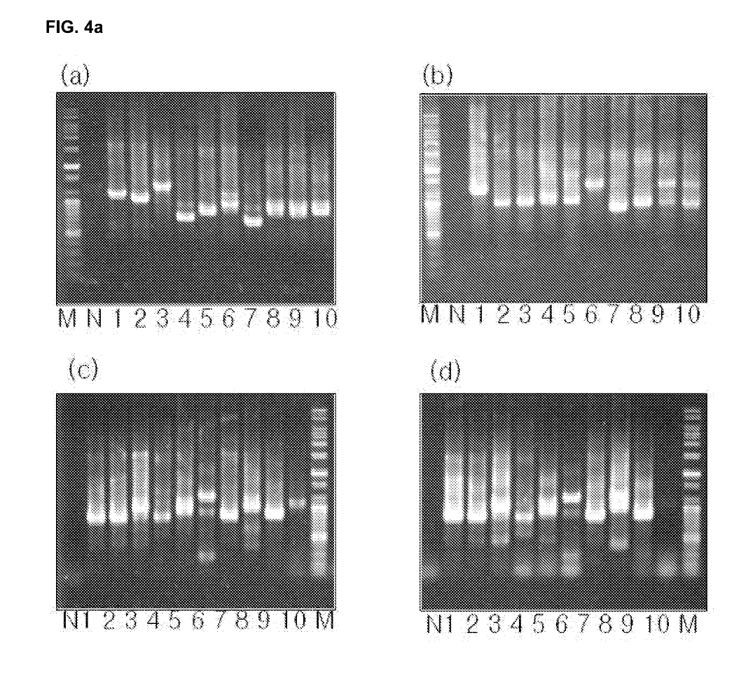

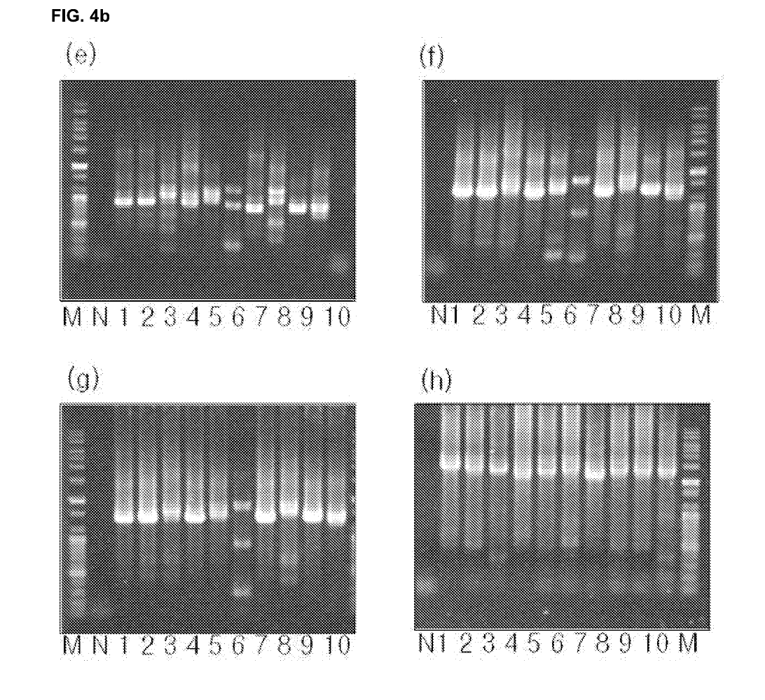

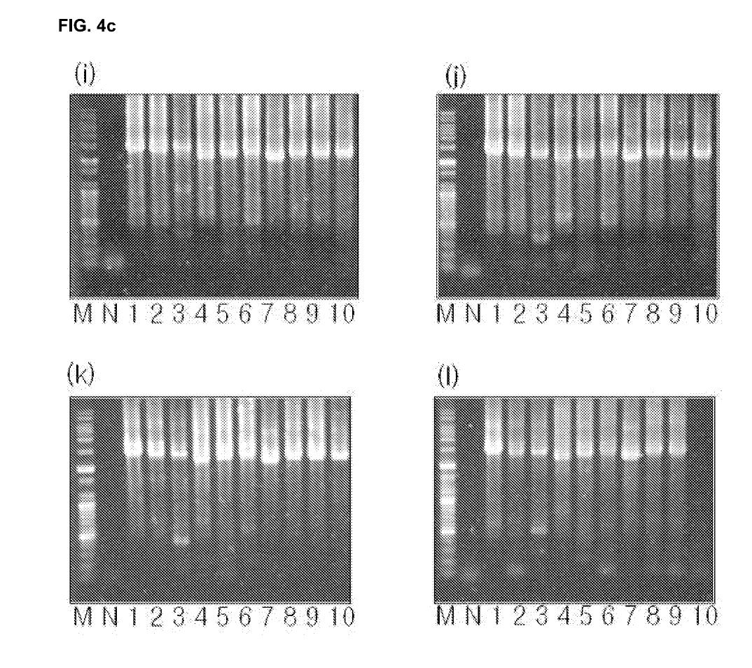

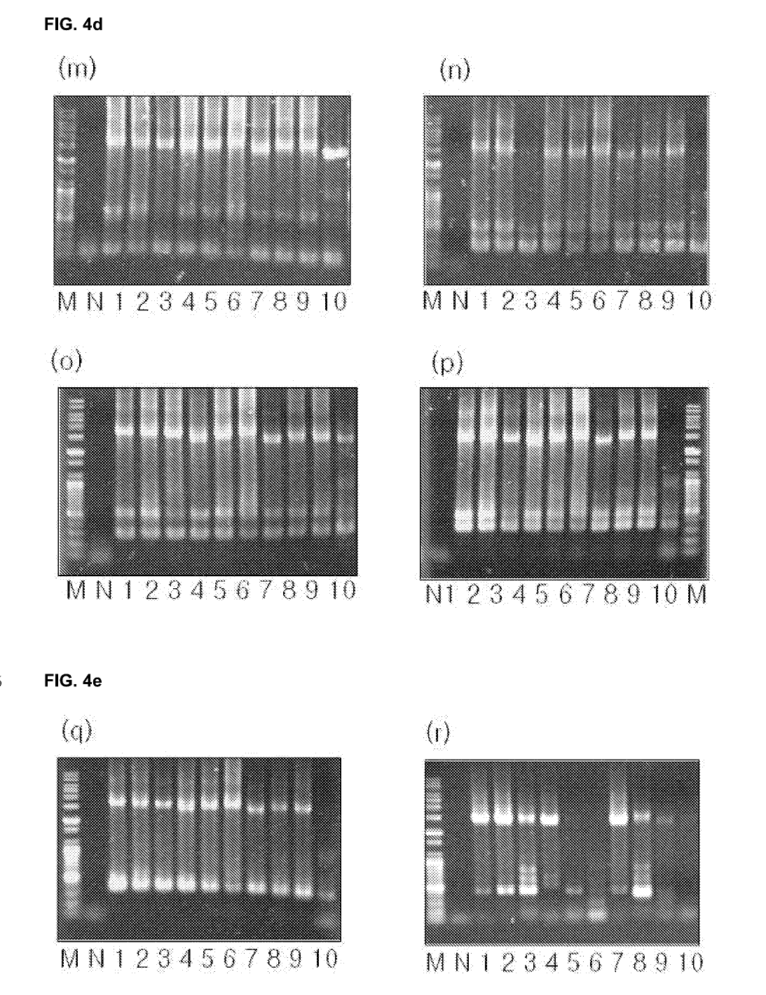

FIG. 4 depicts the result of PCR amplification with a pair of primers designed by using the bacterial-specific nucleotide sequence of the present invention. FIG. 4a to 4r illustrate the PCR amplification with the forward primers 16S-1387F designed by using 16S rDNA and the reverse primers (temporary SEQ NO: 42, 46, 48, 49, 54, 64, 70, 90, 91, 93, 94, 99, 105, 115, 117, 120, 122, 132) designed by using the 23S rDNA of the present invention to detect the presence of microorganism orderly. In all FIGs, lane 1 is the PCR product of Acinetobacter baumannii; lane 2, Aeromonas salmonicida; lane 3, Bacteroides forsythus; lane 4, Clostridium difficile; lane 5, Legionella pneumophilia; lane 6, Morganella morganii; lane 7, Porphyromanas asaccharolytica; lane 8, Proteus mirabilis; lane 9, Mycobacterium tuberculosis; and lane 10, Mycoplasma pneumoniae.

In a preferred embodiment of the PCR method of the present invention, each PCR product of specific bacterial genus is analyzed in Step (2) by using one or more pairs of proper primers. The PCR is performed in a standard strain by using the bacterial genus-specific primers for the amplification described in Example 1.

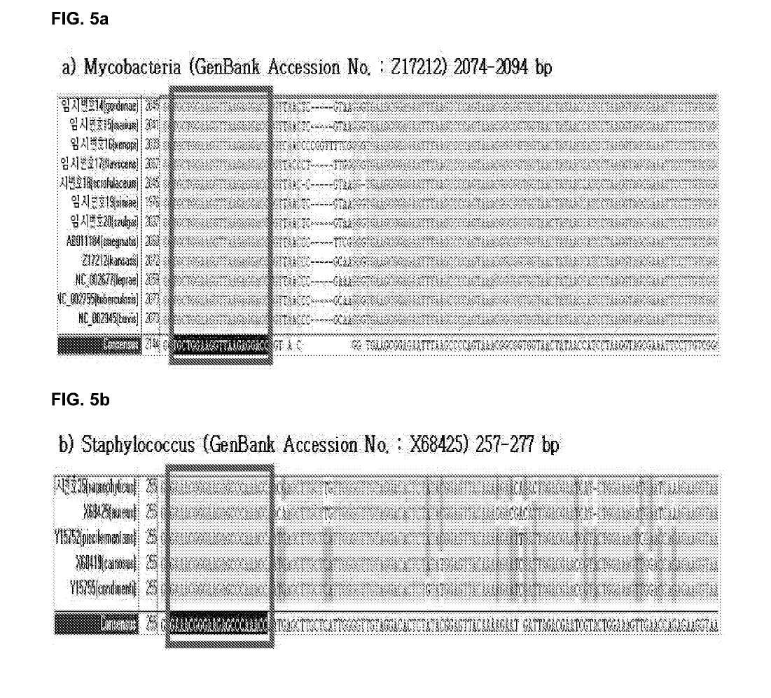

FIG. 5 depicts the multiple alignment of the 23S rDNA gene in the nucleotide sequence of the present invention and the nucleotide sequence already disclosed to design a bacterial genus-specific primer. FIG. 5a depicts the nucleotide sequences of each Mycobacteria sp. in the 23S rDNA gene and FIG. 5b depicts the nucleotide sequences of each Staphylococcus sp. in the 23S rDNA gene to design genus-specific primers and probes.

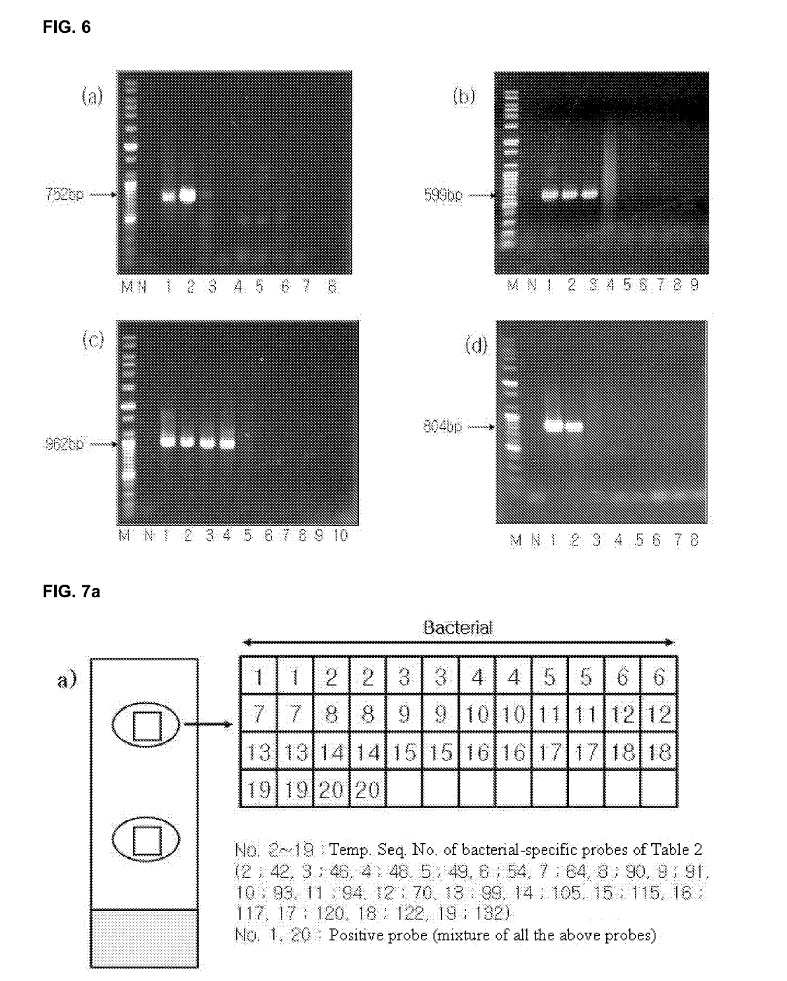

FIG. 6a depicts the PCR amplification of Aeromonas 23S rDNA target sequences with a pair of specific primers of temporary SEQ NO: 199 and SEQ ID NO: 207. Lane 1 is the 752 bp PCR product specific for Aeromonas sp. by using Aeromonas hydrophila as a template; lane 2, Aeromonas salmonicida; lane 3, Mycobacterium xenopi; lane 4, Mycobacterium falconis; lane 5, Streptococcus anginosus; lane 6, Enterococcus faecalis; lane 7, human blood DNA; and lane 8, Hepatitis B virus DNA.

FIG. 6b depicts the PCR amplification of Enterococcus 23S rDNA target sequences with a pair of specific primers of temporary SEQ NO: 699 and SEQ ID NO: 701. Lane 1 is the 599 bp PCR product specific for Enterococcus sp. by using Enterococcus faecalis as a template; lane 2, Enterococcus faecium; lane 3, Enterococcus hirae; lane 4, Aeromonas hydrophila; lane 5, Mycobacterium xenopi; lane 6, Mycobacterium falconis; lane 7, Streptococcus anginosus; lane 8, human blood DNA; and lane 9, Hepatitis B virus DNA. FIG. 6c depicts the PCR amplification of Mycobacteria 23S rDNA target sequences with a pair of specific primers of temporary SEQ NO: 875 and SEQ ID NO: 880. Lane 1 is the 962 bp PCR product specific for Mycobacteria sp. by using Mycobacterium xenopi as a template; lane 2, Mycobacterium flavescence; lane 3, Mycobacterium simiae; lane 4, Mycobacterium tuberculosis; lane 5, Aeromonas hydrophila; lane 6, Mycobacterium falconis; lane 7, Streptococcus anginosus; lane 8, Enterococcus faecalis; lane 9, human blood DNA; and Hepatitis B virus DNA. FIG. 6d depicts the PCR amplification of Streptococcus 23S rDNA target sequences with a pair of specific primers of temporary SEQ NO: 1289 and SEQ ID NO: 1291. Lane 1 is the 804 bp PCR product specific for Streptococcus sp. by using Streptococcus anginosus; lane 2, Streptococcus bovis; lane 3, Aeromonas hydrophila; lane 4, Mycobacterium falconis; lane 5, Mycobacterium xenopi; lane 6, Enterococcus faecalis; lane 7, human blood DNA; and lane 8, Hepatitis B virus DNA.

In a preferred embodiment of the microarray of the present invention, the probes attached onto a substrate have a feature to comprise various kinds in a proper combination for Step (3). Preferably, the probes are optimized to hybridize onto the target region at a time, if reacted and washed under the same condition to detect the presence of microorganism and identify a bacterial genus at a time.

In order to the object of the present invention, the microarray comprising a set of probes attached onto a substrate to detect the presence of microorganism and identify a bacterial genus and species of pathogens that enables a differential diagnosis at a time from a specimen rapidly and exactly, is provided.

In the present invention, “probe” refers to a single-stranded oligonucleotide containing the complementary sequences to a target gene. The oligonucleotides of the present invention can be sense, antisense and complementary sequences selected among all the nucleotide sequences described in the Sequence List, if hybridizing any one of strands of the target gene. The oligonucleotide used as a probe can contain a functional group that does not affect the substantial property for the hybridization. Preferably, the oligonucleotide can be selected among deoxynucleotide (DNA), ribonucleotide (RNA), peptide nucleotide (PNA), locked nucleotide (LNA), dihexynucleotide (HNA), inosine and other modified nucleic acids. In principle, the oligonucleotide can be one or more sequences selected among SEQ ID NO: 1 to 19 or their complementary sequences and contains one or more bacterial-specific sequences. The oligonucleotide can be one or more sequences selected among SEQ ID NO: 20 to 189 or their complementary sequences and contains one or more bacterial genus-specific sequences.

In the present invention, “microorganism” refers to a bacterium and other environmental bacteria causing infectious diseases.

The nucleotide sequences of novel oligonucleotides for a primer and probe that detects the presence of microorganism and identifies a bacterial genus in the present invention are indicated by temporary SEQ NOS, for convenience. In the temporary SEQ NOS, the oligonucleotides described in claims are indicated by regular SEQ ID NOS. The correlation of temporary SEQ NOS and regular SEQ ID NOS is summarized in Table 1. The nucleotide sequences of novel oligonucleotides for a primer and probe to detect the presence of microorganism and identify a bacterial genus in the present invention are summarized in Table 2 and Table 3.

TABLE 1
Correlation of temporary SEQ NOS and regular SEQ ID NOS mentioned in the
invention (regular SEQ ID NOS/temporary SEQ NOS).

| | | | | | | | | | |
|---|---|---|---|---|---|---|---|---|---|
| 1/42 | 2/46 | 3/49 | 4/54 | 5/64 | 6/70 | 7/81 | 8/83 | 9/90 | 10/91 |
| 11/92 | 12/93 | 13/94 | 14/99 | 15/105 | 16/113 | 17/115 | 18/120 | 19/132 | |
| 20/138 | 21/166 | 22/182 | 23/197 | 24/199 | 25/206 | 26/207 | 27/214 | 28/216 | 29/217 |
| 30/221 | 31/222 | 32/224 | 33/225 | 34/228 | 35/234 | 36/240 | 37/255 | 38/278 | 39/284 |
| 40/292 | 41/306 | 42/343 | 43/369 | 44/379 | 45/387 | 46/449 | 47/476 | 48/496 | 49/504 |
| 50/509 | 51/512 | 52/517 | 53/525 | 54/534 | 55/554 | 56/587 | 57/588 | 58/603 | 59/630 |
| 60/636 | 61/637 | 62/638 | 63/642 | 64/648 | 65/653 | 66/654 | 67/656 | 68/659 | 69/663 |
| 70/664 | 71/667 | 72/674 | 73/692 | 74/695 | 75/698 | 76/699 | 77/700 | 78/701 | 79/702 |
| 80/703 | 81/706 | 82/707 | 83/712 | 84/720 | 85/723 | 86/725 | 87/726 | 88/729 | 89/730 |
| 90/731 | 91/732 | 92/734 | 93/738 | 94/741 | 95/743 | 96/750 | 97/754 | 98/755 | 99/756 |
| 100/757 | 101/758 | 102/759 | 103/767 | 104/780 | 105/791 | 106/799 | 107/811 | 108/816 | 109/825 |
| 110/832 | 111/841 | 112/842 | 113/850 | 114/852 | 115/854 | 116/859 | 117/863 | 118/872 | 119/874 |
| 120/875 | 121/878 | 122/879 | 123/880 | 124/881 | 125/882 | 126/884 | 127/888 | 128/889 | 129/890 |
| 130/897 | 131/910 | 132/929 | 133/947 | 134/951 | 135/958 | 136/966 | 137/991 | 138/1070 | 139/1087 |
| 140/1111 | 141/1128 | 142/1132 | 143/1172 | 144/1201 | 145/1222 | 146/1223 | 147/1224 | 148/1225 | 149/1228 |
| 150/1233 | 151/1235 | 152/1240 | 153/1241 | 154/1244 | 155/1245 | 156/1247 | 157/1253 | 158/1254 | 159/1255 |
| 160/1256 | 161/1260 | 162/1261 | 163/1264 | 164/1267 | 165/1268 | 166/1274 | 167/1275 | 168/1279 | 169/1281 |
| 170/1286 | 171/1289 | 172/1292 | 173/1293 | 174/1295 | 175/1297 | 176/1298 | 177/1312 | 178/1350 | 179/1382 |
| 180/1404 | 181/1456 | 182/1469 | 183/1475 | 184/1476 | 185/1478 | 186/1479 | 187/1480 | 188/1483 | 189/1484 |

TABLE 2

Novel bacterial-specific primers/probes

| | | | | |
|---|---|---|---|---|
| | Primer/ | * Target | | Temp. |
| Target Gene | Probe | region | Base Sequence | Seq.No. |

| | | | | |
|---|---|---|---|---|
| 23S rDNA | 23S-128 | 100-128 | GATTCCCGAATGGGGAAACCCA | 38 |
| | 23S-205 | 186-205 | GAACTGAAACATCTAAGTAC | 39 |
| | 23S-250 | 229-250 | GATTCCCTGAGTAGCGGCGAGC | 40 |
| | 23S-253 | 232-253 | TCCCTGAGTAGCGGCGAGCGAA | 41 |
| | 23S-389 | 370-389 | TANGGCGGGACACGTGAAAT | 42 |
| | 23S-399 | 376-399 | GGGACACGTGAAATCCTGTCTGAA | 43 |
| | 23S-431 | 410-431 | CCATCCTCCAAGGCTAAATACT | 44 |
| | 23S-457 | 438-457 | AGTGACCGATAGTGAACNAGTA | 45 |
| | 23S-459 | 441-459 | CCGATAGTGAACCAGTACC | 46 |
| | 23S-461 | 440-461 | ACCGATAGTGAACCAGTACCAG | 47 |
| | 23S-469 | 450-469 | AACCAGTACCGTGAGGGAAA | 48 |
| | 23S-471 | 452-471 | CCAGTACCGTGAGGGAAAGG | 49 |
| | 23S-471-1 | 446-471 | CCAGTACCAGTACCGTGAGGGAAAGG | 50 |
| | 23S-484 | 464-484 | GGGAAAGGCGAAAAGAACCCC | 51 |
| | 23S-506 | 486-506 | GCGAGGGGAGTGAAAGAGAAC | 52 |
| | 23S-515 | 489-515 | AGGGGAGTGAAATAGAACCTGAAACCC | 53 |
| | 23S-520 | 501-520 | NAGAACCTGAAACCGTGTGC | 54 |
| | 23S-573 | 555-573 | GCGTGCCTTTTGTAGAATG | 55 |
| | 23S-633 | 617-633 | AGCCGTAGGGAAACCGA | 56 |
| | 23S-647 | 626-647 | GAAAGCGAGTCTGAATAGGGCG | 57 |
| | 23S-682 | 663-682 | TAGACCCGAAACCNGGTGAT | 58 |
| | 23S-736 | 718-736 | ASTGGAGGACCGAACCSAC | 59 |
| | 23S-782 | 763-782 | TGTGGGTAGGGGTGAAAGGC | 60 |
| | 23S-817 | 794-817 | GGAGATAGCTGGTTCTCSCCGAAA | 61 |
| | 23S-867 | 351-867 | GGGGGTAGAGCACTGTT | 62 |
| | 23S-988 | 969-988 | ANAGGGAAACANCCCAGACC | 63 |
| | 23S-991 | 972-991 | GGGAAACAACCCAGACCGCC | 64 |
| | 23S-1003 | 981-1003 | CCCAGACCGCCAGCTAAGGTCCC | 65 |
| | 23S-1006 | 985-1006 | GACCGCCAGCTAAGGTCCCAAA | 66 |
| | 23S-1008 | 990-1008 | CCAGCTAAGGTCCCCAAAT | 67 |
| | 23S-1034 | 1012-1034 | GTGTAAGTGGGAAAGGATGTGGG | 68 |
| | 23S-1063 | 1042-1063 | AGACAGCGAGGATGTTGGCTTA | 69 |
| | 23S-1075 | 1055-1075 | GTTGGCTTAGAAGCAGCCATC | 70 |
| | 23S-1110 | 1087-1110 | GCGTAATAGCTCACTGGGTCGAGT | 71 |
| | 23S-1137 | 1113-1137 | CCTGCGCGGAAGATGTAGCGGGGCT | 72 |
| | 23S-1198 | 1181-1198 | GGTAGGGGAGCGTTCGT | 73 |
| | 23S-1342 | 1320-1342 | GTGGTCGGCGCAGGGTGAGTCGG | 74 |
| | 23S-1364 | 1345-1369 | CCTAAGGCGAGGCCGAVAKGCGTAG | 75 |
| | 23S-1376 | 1352-1376 | CGAGGCCGAAAGGCGTAGGCGATGG | 76 |
| | 23S-1434 | 1411-1434 | GCGATGGGGGGACGGAGVAGGGTA | 77 |
| | 23S-1574 | 1554-1574 | GCTGCCAAGAAAAGCCTCTAA | 78 |
| | 23S-1617 | 1593-1617 | CCGTACCGCAAACCGACACAGGTGG | 79 |
| | 23S-1648 | 1625-1648 | GAGAATACTAAGGCGCTTGAGAGA | 80 |
| | 23S-1665 | 1643-1665 | GAGAGAACTCGGGTGAAGGAACT | 81 |
| | 23S-1675 | 1655-1675 | GTKAAGGAACTCGGCAAAATG | 82 |
| | 23S-1697 | 1679-1697 | CCGTAACTTCGGGAGAAGG | 83 |
| | 23S-1786 | 1764-1786 | GCGACTGTTTAGGAAAAACACAG | 84 |
| | 23S-1797 | 1777-1797 | AAAAACACAGCACTCTGCAAA | 85 |
| | 23S-1824 | 1808-1824 | GACGTATAGGGTGTGAC | 86 |
| | 23S-1834 | 1812-1834 | TATAGGGTGTGACGCCTGCCCGG | 87 |
| | 23S-1848 | 1821-1848 | TGACGCCTGCCCGGTGCGGGAAGGTTA | 88 |
| | 23S-1903 | 1881-1903 | ATGGAAGCCCCGGTAAACGGCGG | 89 |
| | 23S-1906 | 1890-1906 | CCVGTAAACGGCGGCCG | 90 |
| | 23S-1921 | 1900-1921 | CGGCGGCCGTAACTATAACGGTCC | 91 |
| | 23S-1931 | 1910-1931 | CTATAACGGTCCTAAGGTAGCG | 92 |
| | 23S-1941 | 1921-1941 | CTAAGGTAGCGAAATTCCTTG | 93 |
| | 23S-1961 | 1941-1961 | GTCGGGTAAGTTCCGACCTGC | 94 |
| | 23S-1966 | 1943-1966 | CGGGTAAGTTCCGACCGGCACGAA | 95 |
| | 23S-1970 | 1947-1970 | TAAGTTCCGACCTGCACGAATGGC | 96 |
| | 23S-2013 | 1994-2013 | ACGGGAGACTCGGTGAAATT | 97 |
| | 23S-2018 | 1998-2018 | GAGACTCGGTGAAATTGGAGT | 98 |
| | 23S-2069 | 2049-2069 | GACGGAAAGACCCCGTGAACC | 99 |
| | 23S-2075 | 2054-2075 | AAAGACCCCGTGGAGCTTTACT | 100 |
| | 23S-2125 | 2105-2125 | TGTGTAGGATAGGTGGGAGGC | 101 |
| | 23S-2131 | 2110-2131 | AGGATAGGTGGGAGGCTTTGAA | 102 |
| | 23S-2177 | 2162-2177 | TTGAAATACCACCCTT | 103 |
| | 23S-2250 | 2230-2250 | GGTGGGCAGTTTGACTGGGGC | 104 |
| | 23S-2252 | 2232-2252 | TGGGTAGTTTGACTGGGGCGG | 105 |
| | 23S-2258 | 2233-2258 | GGGGAGTTTGACTGGGGCGGTCGCCT | 106 |
| | 23S-2260 | 2237-2260 | AGTTTGACTGGGGCGGTCGCTCC | 107 |
| | 23S-2264 | 2246-2264 | GGGGCGGTCGCCTCCTAAA | 108 |
| | 23S-2288 | 2267-2288 | GTAACGGAGGCGCCCGAAGGTT | 109 |
| | 23S-2351 | 2336-2351 | AGCTTGACTGCGAGAC | 110 |
| | 23S-2375 | 2354-2375 | ACAAGTCGAGCAGGGGCGAAAG | 111 |
| | 23S-2399 | 2378-2399 | GGGCGTAGTGATCCGGTGGTTC | 112 |
| | 23S-2401 | 2383-2401 | TAGTGATCCGGTGGTTCTG | 113 |
| | 23S-2425 | 2401-2425 | GGATGGAAGGGCCATCGCTCAACGG | 114 |
| | 23S-2431 | 2412-2431 | CCATCGCTCAACGGATAAAA | 115 |
| | 23S-2436 | 2416-2436 | CGCTCAACGGATAAAAGGTAC | 116 |
| | 23S-2443 | 2423-2443 | CGGATAAAAGGTACTCCGGGG | 117 |
| | 23S-2456 | 2434-2456 | TACGCCGGGGATAACAGGCTGAT | 118 |
| | 23S-2481 | 2461-2481 | CCCAAGAGTTCATATCGACGG | 119 |
| | 23S-2504 | 2486-2504 | GTTTGGCACCTCGATGTCG | 120 |
| | 23S-2511 | 2486-2511 | GTTTGGCACCTCGATGTCGGCTCATC | 121 |
| | 23S-2517 | 2497-2517 | CGATGTCGGCTCATCACATCC | 122 |
| | 23S-2525 | 2503-2525 | CGGCTCATCGCATCCTGGGGCTG | 123 |
| | 23S-2536 | 2513-2536 | CATCCTGGGGCTGGAGTGGGTCCC | 124 |
| | 23S-2542 | 2519-2542 | GGGGCTGGAGTGGGTCCCAAGGGT | 125 |
| | 23S-2551 | 2531-2551 | GGTCCCAAGGGTCCGGCTGTT | 126 |
| | 23S-2563 | 2545-2563 | GGCTGTTCGCCATTTAAAG | 127 |
| | 23S-2588 | 2565-2588 | GGTACGCGAGCTGGGTTSAGAACG | 128 |
| | 23S-2596 | 2572-2596 | GAGCTGGGTTGAGAACGTCGTGAGA | 129 |
| | 23S-2599 | 2577-2599 | GGGTTCAGAACGTCGTGAGACAG | 130 |
| | 23S-2604 | 2583-2604 | AGAACGTCGTGAGACAGTTCGG | 131 |
| | 23S-2607 | 2588-2607 | GTCGTGAGACAGTTCGGTCC | 132 |
| | 23S-2612 | 2589-2612 | TCGTGAGACAGTTCGGTCCCTATC | 133 |
| | 23S-2665 | 2647-2665 | CGTAGTACGAGAGGACCGG | 134 |
| | 23S-2756 | 2733-2756 | GATAASSGCTGAAAGCATCTAAGC | 135 |

Target regions of standard strain: E. Coli (GenBank Accession No.: AJ278710) is referred for nucleotide sequence analysis

Code names of mixed bases: M: A+C, W: A+T, Y: C+T, R: A+G, K: G+T, S: G+C, V: G+A+C, N: A+G+C+T

TABLE 3

Novel bacterial genus-specific primers/probes for differential diagnosis

| | | | | | |
|---|---|---|---|---|---|
| Target | | Primer/ | Target | | Temp. |
| Gene | Gene Name | Probe | Region | Base Sequence | Seq.No. |

| | | | | | |
|---|---|---|---|---|---|
| 23S | Acinetobacter | Acin-35 | 11-35 | TTAAGTGCATGTGGTGGATGCCTTG | 136 |
| rDNA | | Acin-144 | 118-144 | AATGGGGGAACCCACCTACTTTAAGGT | 137 |
| | | Acin-148 | 127-148 | ACCCACCTACTTTAAGGTAGGT | 138 |
| | | Acin-186 | 161-186 | ATACATAGTGTTGCAAGGCGAACGAG | 139 |
| | | Acin-217 | 196-217 | AACATCTCAGTAGCCTTAGGAA | 140 |
| | | Acin-220 | 201-220 | CTCAGTACCCTTAGGAAAAG | 141 |
| | | Acin-309 | 280-309 | TGTGTGTTTTAGTGGAACGCTCTGGGAAGT | 142 |
| | | Acin-314 | 295-314 | AACGCTCTGGGAAGTGCGAA | 143 |
| | | Acin-321 | 299-321 | CTCTGGGAAGTGCGAACGTAGAG | 144 |
| | | Acin-348 | 325-348 | GATATTCCCGTACACGAAAGGGCA | 145 |
| | | Acin-358 | 333-358 | CGTACACGAAAGGGCACACATAATGA | 146 |
| | | Acin-365 | 343-365 | AGGGCACACATAATGATGACGAG | 147 |
| | | Acin-374 | 351-374 | CATAATGATGACGAGTAGGGCGAG | 148 |
| | | Acin-395 | 371-395 | CGAGGCACGTGAAACCTTGTCTGAA | 149 |
| | | Acin-506 | 478-506 | CCCTGTGAGGGGAGTGAAATAGATCCTGA | 150 |
| | | Acin-594 | 575-594 | GCGACTTATATTCAGTAGCG | 151 |
| | | Acin-627 | 604-627 | GTATAGGGGAGCCGTAGAGAAATC | 152 |
| | | Acin-646 | 624-646 | AATCGAGTCTTAATAGGGCGTTT | 153 |
| | | Acin-661 | 635-661 | AATAGGGCGTTTAGTTGCTGGGTATAG | 154 |
| | | Acin-731 | 712-731 | TAACTGGAGGACCGAACCCA | 155 |
| | | Acin-850 | 827-850 | CGCCTCGGACGAATACCATAGGGG | 156 |
| | | Acin-860 | 833-860 | GGACGAATACCATAGGGGGTAGAGCACT | 157 |
| | | Acin-865 | 843-865 | CATAGGGGGTAGAGCACTGTTTC | 158 |
| | | Acin-918 | 899-918 | GCAAACTCCGAATACCTATG | 159 |
| | | Acin-923 | 904-323 | CTCCGAATACCTATGAGTAC | 160 |
| | | Acin-951 | 932-951 | AGACAGACTGCGGGTGCTAA | 161 |
| | | Acin-993 | 964-993 | AGAGGAAAACAATCCAGAGCGCCAGCTAAG | 162 |
| | | Acin-1008 | 984-1008 | GCCAGCTAAGGCCCCAAAATCATAG | 163 |
| | | Acin-1141 | 1122-1141 | GTAACGGGGCTAAAACTATG | 164 |
| | | Acin-1150 | 1124-1150 | AACGGGGCTAAAACTATGTGCCGAAGC | 165 |
| | | Acin-1154 | 1133-1154 | AAAACTATGTGCCGAAGCTGCG | 166 |
| | | Acin-1157 | 1138-1157 | TATGTGCCGAAGCTGCGGAT | 167 |
| | | Acin-1275 | 1249-1275 | GACGTGAGTAACGACAAAACGGGTGAA | 168 |
| | | Acin-1286 | 1260-1286 | CGACAAAACGGGTGAAAAACCCGTTCG | 169 |
| | | Acin-1303 | 1283-1303 | TTCGCCGAAAGACCAAGGGTT | 170 |
| | | Acin-1313 | 1287-1313 | CCGAAAGACCAAGGGTTCCAGTCCAAC | 171 |
| | | Acin-1330 | 1311-1330 | AACGTTAATCGGGGCTGGGT | 172 |
| | | Acin-1334 | 1315-1334 | TTAATCGGGGCTGGGTGAGT | 173 |
| | | Acin-1413 | 1394-1413 | CTTGTGTGTAATGCGATGAG | 174 |
| | | Acin-1424 | 1396-1424 | TCTGTGTAATGCGATGAGAGGACGGAGAA | 175 |
| | | Acin-1517 | 1498-1517 | CTATGCTGAGATCTGATAGC | 176 |
| | | Acin-1524 | 1501-1524 | TGCTGAGATCTGATAGCAAGCTGT | 177 |
| | | Acin-1576 | 1556-1576 | TCCAGGAAAAGTCTCTAAGCT | 178 |
| | | Acin-1589 | 1570-1589 | CTAAGCTTCAGTTACACAGG | 179 |
| | | Acin-1594 | 1574-1594 | GCTTCAGTTACACAGGAATCG | 180 |
| | | Acin-1630 | 1609-1630 | ACAGGTGGTGAGGTCGAGTAGA | 181 |
| | | Acin-1636 | 1614-1636 | TGGTCAGGTCGAGTAGACCAAGG | 182 |
| | | Acin-1641 | 1622-1641 | TCGAGTAGACCAAGGCGCTT | 183 |
| | | Acin-1653 | 1624-1653 | GAGTAGACCAAGGCGCTTGAGAGAACTCTG | 184 |
| | | Acin-1705 | 1685-1705 | TTCGGGAGAAGGTACGCTGTT | 185 |
| | | Acin-1774 | 1752-1774 | CAGGCCGCTGCAACTGTTTATTA | 186 |
| | | Acin-1980 | 1955-1980 | GCACGAATGGCATAATGATGGCGGCG | 187 |
| | | Acin-1986 | 1965-1986 | CATAATGATGGCGGCGCTGTCT | 188 |
| | | Acin-2012 | 1986-2012 | TCCAGCAGAGGCTCAGTGAAATCGAAA | 189 |
| | | Acin-2016 | 1995-2016 | GGCTCAGTGAAATCGAAATCGC | 190 |
| | | Acin-2112 | 2093-2112 | CCTTACTTGTGTAGGATAGG | 191 |
| | | Acin-2119 | 2096-2119 | TACTTGTGTAGGATAGGTGGGAGG | 192 |
| | | Acin-2317 | 2292-2317 | GTGGTCGGAAATCACGCGTAGAGTAT | 193 |
| | | Acin-2325 | 2303-2325 | TCACGCGTAGAGTATAAAGGCAA | 194 |
| | | Acin-2355 | 2332-2355 | GCTTAACTGCGAGACCCACAAGTC | 195 |
| | | Acin-2364 | 2345-2364 | ACCCACAAGTCGAGCAGGTA | 196 |
| | Aeromonas | Aer-315 | 296-315 | AGTGGAAYGGTCCTGGAAAG | 197 |
| | | Aer-543 | 524-543 | TACAAGCAGTGGGAGCCCTT | 198 |
| | | Aer-660 | 640-660 | CTGGGCGTCTAGTTGCAAGGT | 199 |
| | | Aer-750 | 722-750 | AGGACCGAACCCACTAACGTTGCAAAGTT | 200 |
| | | Aer-1153 | 1134-1153 | CTCAAACCAGGCACCGAAGC | 201 |
| | | Aer-1274 | 1254-1274 | CGTAAGTAACGATAAAGGGGG | 202 |
| | | Aer-1278 | 1257-1278 | AAGTAACGATAAAGGGGGTGAA | 203 |
| | | Aer-1287 | 1262-1287 | ACGATAAAGGGGGTGAAAAGCCTCCT | 204 |
| | | Aer-1398 | 1378-1398 | GCAGGTTAATATTCCTGCACG | 205 |
| | | Aer-1400 | 1381-1400 | GGTTAATATTCCTGCACGAC | 206 |
| | | Aer-1411 | 1392-1411 | CTGCACGACTTGTAATTGCG | 207 |
| | | Aer-1566 | 1540-1566 | CCATTGATGCCCTGCTTCCAGGAAAAG | 208 |
| | | Aer-1761 | 1742-1761 | TCGGAGTGACCAGATGGCTG | 209 |
| | | Aer-1776 | 1750-1776 | ACCAGATGGCTGGGACTGTTTATCAAA | 210 |
| | | Aer-2009 | 1984-2009 | TCCACCCGAGACTCAGTGAAATCGAA | 211 |
| | | Aer-2194 | 2175-2194 | GTTTGATGTTCTAACGCAGG | 212 |
| | | Aer-2353 | 2329-2353 | CAGCTTAACTGCGAGACGGACAGGT | 213 |
| | | Aer-2636 | 2610-2636 | CGTGGGCGTTGGATGATTGAAGGGAGT | 214 |
| | | Aer-2691 | 2671-2691 | CTCTGGTGTTCGGGTTGTCAC | 215 |
| | | Aer-2720 | 2700-2720 | CACTGCCCGGTAGCTAAGTTC | 216 |
| | Bacillus | Baci-143 | 122-143 | GAGGAAGAGAAAGCAAATGCGA | 217 |
| | | Baci-194 | 175-194 | AGCCCAAACCAAGAGGCTTG | 218 |
| | | Baci-202 | 181-202 | AACCAAGAGGCTTGCCTCTTGG | 219 |
| | | Baci-214 | 195-214 | CCTCTTGGGGTTGTAGGACA | 220 |
| | | Baci-688 | 662-688 | TCAGGTAACACTGAATGGAGGCCCGAA | 221 |
| | | Baci-818 | 799-818 | AAGAGTCTTGGAGGTAGAGC | 222 |
| | | Baci-870 | 849-870 | ACCGAATTCAGTCAAACTCCGA | 223 |
| | | Baci-869 | 850-869 | CCGAATTCAGTCAAACTCCG | 224 |
| | | Baci-1087 | 1067-1087 | GACTCTGCGCCGAAAATGTAC | 225 |
| | | Baci-1089 | 1063-1089 | GAGTGACTCTGCGCCGAAAATGTACCG | 226 |
| | | Baci-1365 | 1346-1365 | ATTCCTGTACCACCTCYTYA | 227 |
| | | Baci-2106 | 2087-2106 | AGCGCTAGCTTCGGTGGAGG | 228 |
| | Bacterioides | Bact-4985599 | 4985579-4985599 | CCTTTGATCCAGAGATTTCCG | 229 |
| | | Bact-4985613 | 4985584-4985613 | GATCCAGAGATTTCCGAATGGGACAACCCG | 230 |
| | | Bact-4985620 | 4985599-4985620 | GAATGGGAGAACCCGGCATTCT | 231 |
| | | Bact-4985632 | 4985604-4985632 | GGACAACCCGGCATTCTGAAGGAATGTCA | 232 |
| | | Bact-4985638 | 4985619-4985638 | CTGAAGGAATGTCATCCATC | 233 |
| | | Bact-4985650 | 4985631-4985650 | CATCCATCTTTGATGGAAGC | 234 |
| | | Bact-4985746 | 4985724-4985746 | CGAACGGGGAATAGCCCAAACCA | 235 |
| | | Bact-4985780 | 4985758-4985780 | GCATGTGTGGGGTTGTAGGACCA | 236 |
| | | Bact-4985784 | 4985765-4985784 | TGGGGTTGTAGGACCACGAT | 237 |
| | | Bact-4985793 | 4985767-4985793 | GGGTTGTAGGACCACGATGTCGCAAGA | 238 |
| | | Bact-4985800 | 4985776-4985800 | GACCACGATGTCGCAAGACATTTGA | 239 |
| | | Bact-4985804 | 4985785-4985804 | GTCGCAAGACATTTGATGAG | 240 |
| | | Bact-4985818 | 4985798-4985818 | TGATGAGTAGAATCCTCTGGA | 241 |
| | | Bact-4985821 | 4985802-4985821 | GAGTAGAATCCTCTGGAAAG | 242 |
| | | Bact-4985830 | 4985809-4985830 | ATCCTCTGGAAAGTTGAACCAT | 243 |
| | | Bact-4985836 | 4985816-4985836 | GGAAAGTTGAACCATAGACGG | 244 |
| | | Bact-4985840 | 4985819-4985840 | AAGTTGAACCATAGACGGTGAT | 245 |
| | | Bact-4985889 | 4985862-4985889 | TTAAGCGTAGTGGTATCCTGAGTAGCGC | 246 |
| | | Bact-4985903 | 4985884-4985903 | TAGCGCGGGACACGAGAAAT | 247 |
| | | Bact-4985913 | 4985887-4985913 | CGCGGGACACGAGAAATCTTGCGTGAA | 248 |
| | | Bact-4985915 | 4985896-4985915 | CGAGAAATCTTGCGTGAATC | 249 |
| | | Bact-4985921 | 4985899-4985921 | GAAATCTTGCGTGAATCTGCCGG | 250 |
| | | Bact-4985993 | 4985974-4985993 | GTGAAGGAAAGGTGAAAAGC | 251 |
| | | Bact-4985995 | 4985976-4985995 | GAAGGAAAGGTGAAAAGCAC | 252 |
| | | Bact-4985999 | 4985979-4985999 | GGAAAGGTGAAAAGCACTTCG | 253 |
| | | Bact-4986011 | 4985992-4986011 | GCACTTCGAATAGAAGAGTG | 254 |
| | | Bact-4986023 | 4986004-4986023 | GAAGAGTGAAATAGTCCCTG | 255 |
| | | Bact-4986033 | 4986009-4986033 | GTGAAATAGTCCCTGAAACCGTGCG | 256 |
| | | Bact-4986041 | 4986021-4986041 | CTGAAACCGTGCGCCTACAAG | 257 |
| | | Bact-4986058 | 4986039-4986058 | AAGCGGTCGGAGCTGCTTAA | 258 |
| | | Bact-4986071 | 4986044-4986071 | GTCGGAGCTGCTTAAGCAGTGACGGCGT | 259 |
| | | Bact-4986115 | 4986089-4986115 | CCTACGAGTTACTTTTTCCGGCAAGGT | 260 |
| | | Bact-4986128 | 4986100-4986128 | CTTTTTCCGGCAAGGTTAAGCATCTTGAG | 261 |
| | | Bact-4986143 | 4986117-4986143 | AAGCATCTTGAGATGTGCAGCCGAAGC | 262 |
| | | Bact-4986148 | 4986127-4986148 | AGATGTGCAGCCGAAGCGAAAG | 263 |
| | | Bact-4986164 | 4986138-4986164 | CGAAGCGAAAGCGAGTCTGAACAGGGC | 264 |
| | | Bact-4986185 | 4986165-4986185 | GTCGAGTCGGAAGGAGTAGAC | 265 |
| | | Bact-4986257 | 4986234-4986257 | TAACTGGAGGACCGAACCGATAAG | 266 |
| | | Bact-4986265 | 4986241-4986265 | AGGACCGAACCGATAAGCGTTGAAA | 267 |
| | | Bact-4986269 | 4986249-4986269 | ACCGATAAGCGTTGAAAAGCT | 268 |
| | | Bact-4986285 | 4986261-4986285 | TGAAAAGCTTCCGGATGAACTGAGG | 269 |
| | | Bact-4986288 | 4986268-4986288 | CTTCCGGATGAACTGAGGGTG | 270 |
| | | Bact-4986297 | 4986278-4986297 | AACTGAGGGTGGGGGTGAAA | 271 |
| | | Bact-4986309 | 4986285-4986309 | GGTGGGGGTGAAAGGCTAATCAAAC | 272 |
| | | Bact-4986414 | 4986395-4986414 | AGGGCTTCACCGCCTATCAA | 273 |
| | | Bact-4986423 | 4986397-4986423 | GGCTTCACCGCCTATCAAGTCTTGATA | 274 |
| | | Bact-4986439 | 4986417-4986439 | CTTGATAAACTCCGAATGCGCAT | 275 |
| | | Bact-4986447 | 4986423-4986447 | AAACTCCGAATGCGCATTAGTTCTA | 276 |
| | | Bact-4986454 | 4986434-4986454 | GCGCATTAGTTCTATCACAGG | 277 |
| | | Bact-4986466 | 4986441-4986466 | AGTTCTATCACAGGAGTGAGGGCATG | 278 |
| | | Bact-4986503 | 4986482-4986503 | GTCCTAAAGGAGAAGAATCCAG | 279 |
| | | Bact-4986528 | 4986504-4986528 | ACCATCAGCTAAGGTCCCCAAATAA | 280 |
| | | Bact-4986530 | 4986511-4986530 | GCTAAGGTCCCCAAATAAAC | 281 |
| | | Bact-4986565 | 4986546-4986565 | CGAAGTCAGATTGCTAAGAC | 282 |
| | | Bact-4986569 | 4986549-4986569 | AGTCAGATTGCTAAGACAGCT | 283 |
| | | Bact-4986573 | 4986552-4986573 | CAGATTGCTAAGACAGCTAGGA | 284 |
| | | Bact-4986648 | 4986625-4986648 | TCGAGGAGTTTGGCGTGGATAATA | 285 |
| | | Bact-4986657 | 4986634-4986657 | TTGGCGTGGATAATAATCGGGGAT | 286 |
| | | Bact-4986682 | 4986661-4986682 | GTGTTTTACCGAAGCTATGGGA | 287 |
| | | Bact-4986689 | 4986665-4986689 | TTTACCGAAGCTATGGGATCAGTAA | 288 |
| | | Bact-4986747 | 4986719-4986747 | TCGAAGGTGAAGCGTGAGCTTTGCTGGAG | 289 |
| | | Bact-4986752 | 4986733-4936752 | TGAGCTTTGCTGGAGCGTGT | 290 |
| | | Bact-4986759 | 4986737-4986759 | CTTTGCTGGAGCGTGTGGAAAAG | 291 |
| | | Bact-4986766 | 4986742-4986766 | CTGGAGCGTGTGGAAAAGCAAATGT | 292 |
| | | Bact-4986801 | 4986773-4986801 | AAGTAACGATAAAGGGGGTGAGAAACCCC | 293 |
| | | Bact-4986826 | 4986801-4986826 | CCTCGCCGAAAGACTAAGGTTTCCTG | 294 |
| | | Bact-4986837 | 4986814-4386837 | CTAAGGTTTCCTGATCAACGCTAA | 295 |
| | | Bact-4986864 | 4986842-4986864 | ATCAGGGTTAGTCGGGTCCTAAG | 296 |
| | | Bact-4986904 | 4986883-4986904 | GCCGATGGCCAGAACAGGTTAAT | 297 |
| | | Bact-4987018 | 4986999-4987018 | ATCCCAGGCAAATCCGGGAT | 298 |
| | | Bact-4987029 | 4987003-4987029 | CAGGCAAATCCGGGATGAGAGTCGAAC | 299 |
| | | Bact-4987038 | 4987014-4987038 | GGGATGAGAGTCGAACCTGATAGTA | 300 |
| | | Bact-4987072 | 4987050-4987072 | TCGGAACAATCCAATAGTGCGTG | 301 |
| | | Bact-4987100 | 4987077-4987100 | CATACTCCCAAGAAAATCCGCTAA | 302 |
| | | Bact-4987102 | 4987083-4987102 | CCCAAGAAAATCCGCTAAAC | 303 |
| | | Bact-4987142 | 4987122-4987142 | TACCGCAAACGGACACACGTA | 304 |
| | | Bact-4987147 | 4987125-4987147 | CGCAAACGGACACACGTAGTCGG | 305 |
| | | Bact-4987156 | 4987133-4987156 | GACACACGTAGTCGGGTTGAATAT | 306 |
| | | Bact-4987158 | 4987139-4987158 | CGTAGTCGGGTTGAATATAC | 307 |
| | | Bact-4987166 | 4987145-4987166 | CGGGTTGAATATACTAAGGCGC | 308 |
| | | Bact-4987180 | 4987154-4987180 | TATACTAAGGCGCTTGAGTGATTCACG | 309 |
| | | Bact-4987184 | 4987163-4987184 | GCGCTTGAGTGATTCACGGTTA | 310 |
| | | Bact-4987188 | 4987168-4987188 | TGAGTGATTCACGGTTAAGGA | 311 |
| | | Bact-4987190 | 4987171-4987190 | GTGATTCACGGTTAAGGAAC | 312 |
| | | Bact-4987198 | 4987175-4987198 | TTCACGGTTAAGGAACTAGGCAAA | 313 |
| | | Bact-4987209 | 4987185-4987209 | AGGAACTAGGCAAATTGACCCTGTA | 314 |
| | | Bact-4987212 | 4987192-4987212 | AGGAAATTGACCCTGTAACT | 315 |
| | | Bact-4987240 | 4987215-4987240 | GGGATAAAGGGTCCCAACGAGAGTTG | 316 |
| | | Bact-4987257 | 4987233-4987257 | GAGAGTTGGGCGCAGAGAATAGGTC | 317 |
| | | Bact-4987356 | 4987328-4987356 | CCGGTGCTGGAAGGTTAAGAGGAGATGTG | 318 |
| | | Bact-4987389 | 4987366-4987389 | GAAGCATTGAATTGAAGCCCCAGT | 319 |
| | | Bact-4987474 | 4987455-4987474 | GCACGAATGGTGTAATGATC | 320 |
| | | Bact-4987477 | 4987458-4987477 | CGAATGGTGTAATGATCTGG | 321 |
| | | Bact-4987483 | 4987462-4987483 | TGGTGTAATGATCTGGACACTG | 322 |
| | | Bact-4987485 | 4987466-4987485 | GTAATGATCTGGACACTGTC | 323 |
| | | Bact-4987551 | 4987530-4987551 | ATTACCCGCGATGGGACGAAAA | 324 |
| | | Bact-4987559 | 4987540-4987559 | ATGGGACGAAAAGACCCCGT | 325 |
| | | Bact-4987619 | 4987595-4987619 | ATTGATGTGTAGGATAGGCCGGAGA | 326 |
| | | Bact-4987684 | 4987665-4987684 | CGGCCCTTTGATTATTTGAG | 327 |
| | | Bact-4987774 | 4987751-4987774 | AGTAACGGAGGCTTCTAAAGGTGC | 328 |
| | | Bact-4987833 | 4987814-4987833 | ATAAGGGCGCTTGACTGGGA | 329 |
| | | Bact-4987868 | 4987839-4987868 | ACAAGTCGATCAGGTAGGAAACTAGAGCAT | 330 |
| | | Bact-4987914 | 4987885-4987914 | CGTATGGAAGGGACATCGCTCAAAGGATAA | 331 |
| | | Bact-4987950 | 4987926-4987950 | GGGATAACAGGCTGATCCCTCCCAA | 332 |
| | | Bact-4987976 | 4987954-4987976 | CTCATATCGACGGAGGGGTTTGG | 333 |
| | | Bact-4988127 | 4988100-4988127 | TCGTGGGCGTATGAAATTTGCGTGGCTG | 334 |
| | Bordetella | Bord-28 | 4-23 | CAAGCGACTAAGTGCATATGGTGGA | 335 |
| | | Bord-40 | 11-40 | CTAAGTGCATATGGTGGATGCCTTGGCGAT | 336 |
| | | Bord-79 | 52-79 | AAGGACGTAGTAGCCTGCGAAAAGCTGC | 337 |
| | | Bord-123 | 96-123 | AGCATTGATCCGCAGATATCCGAATGGG | 338 |
| | | Bord-155 | 136-155 | AAGCGGTATCCCTGGCTGAA | 339 |
| | | Bord-258 | 238-258 | TAGTGGCGAGCGAAATCGGAA | 340 |
| | | Bord-273 | 245-273 | GAGCGAAATCGGAAGAGCCTTTACGATTT | 341 |
| | | Bord-276 | 254-276 | CGGAAGAGCCTTTACGATTTAGC | 342 |
| | | Bord-295 | 275-295 | GCATTTTGCATAGTCGAACGG | 343 |
| | | Bord-302 | 280-302 | TTGCATAGTCGAACGGAATGGAA | 344 |
| | | Bord-360 | 340-360 | TGCAGAGTGTGGAACTAGGCG | 345 |
| | | Bord-370 | 342-370 | CAGAGTGTGGAACTAGGCGTAAGAGAAGT | 346 |
| | | Bord-375 | 353-375 | ACTAGGCGTAAGAGAAGTAGGGC | 347 |
| | | Bord-381 | 360-381 | GTAAGAGAAGTAGGGCGGGACA | 348 |
| | | Bord-544 | 524-544 | CAGTCGGAGCCTCTTTATGGG | 349 |
| | | Bord-546 | 527-546 | TCGGAGCCTCTTTATGGGGT | 350 |
| | | Bord-550 | 529-550 | GGAGCCTCTTTATGGGGTGACG | 351 |
| | | Bord-556 | 535-556 | TCTTTATGGGGTGACGGCGTAC | 352 |
| | | Bord-742 | 718-742 | AGGACCGAACCCACTAGTGTTGAAA | 353 |
| | | Bord-766 | 738-766 | TGAAAAACTAGGGGATGAGCTGTGGATAG | 354 |
| | | Bord-872 | 843-872 | CAGGGGGTAGAGCACTGTTATGGCTAGGGG | 355 |
| | | Bord-879 | 855-879 | CACTGTTATGGCTAGGGGGTCATGG | 356 |
| | | Bord-900 | 871-900 | GGGTCATGGCGACTTACCAAACCATGGCAA | 357 |
| | | Bord-902 | 883-902 | CTTACCAAACCATGGCAAAC | 358 |
| | | Bord-932 | 911-932 | CCTGCAAGTACAGCTTGGGAGA | 359 |
| | | Bord-936 | 908-936 | ATACCTGCAAGTACAGCTTGGGAGACAGA | 360 |
| | | Bord-1167 | 1148-1167 | CGGGTGTGCACTTTTAGTGC | 361 |
| | | Bord-1372 | 1344-1372 | AGGCAGAGATGCGTAGCTGATGGGAAGCT | 362 |
| | | Bord-1379 | 1356-1379 | GTAGCTGATGGGAAGCTGGTTAAT | 363 |
| | | Bord-1405 | 1384-1405 | CAGCACCGTCGTACAGTGCGAT | 364 |
| | | Bord-1443 | 1420-1443 | CGGAAGGTCATCAGGGTGTTGGAC | 365 |
| | | Bord-1446 | 1426-1446 | GTCATCAGGGTGTTGGACGTC | 366 |
| | | Bord-1455 | 1436-1455 | TGTTGGACGTCCCTGTTGCT | 367 |
| | | Bord-1477 | 1455-1477 | TGCATTGAAGATGGCGCTTAGGC | 368 |
| | | Bord-1498 | 1478-1498 | AAATCCGGGCGCGAGAATCAA | 369 |
| | | Bord-1509 | 1488-1509 | GCGAGAATCAAGGGTGTGGCAC | 370 |
| | | Bord-1512 | 1489-1512 | CGAGAATCAAGGGTGTGGCACGAG | 371 |
| | | Bord-1585 | 1564-1585 | CTTCAGCTGTACGAGACCGTAC | 372 |
| | | Bord-1694 | 1671-1694 | AACTTCGGGAGAAGGTATACCCTG | 373 |
| | | Bord-1706 | 1677-1706 | GGGAGAAGGTATACCCTGGTAGTGTGAAGC | 374 |
| | | Bord-1740 | 1719-1740 | CATGAAGGGGTCGCAGAGAATC | 375 |
| | | Bord-2075 | 2046-2075 | ACTGTAGCTTTGCATTGGACTGTGAACCGG | 376 |
| | | Bord-2078 | 2059-2078 | ATTGGACTGTGAACCGGCCT | 377 |
| | | Bord-2083 | 2064-2083 | ACTGTGAACCGGCCTGTGTA | 378 |
| | | Bord-2123 | 2100-2123 | CAGAACTCGAGTCGCCAGATTCGA | 379 |
| | | Bord-2262 | 2233-2262 | CCAAAGCGTAACGGAGGAGTTCGAAGGTAC | 380 |
| | | Bord-2600 | 2581-2600 | CGTGGGCGTTGGATACTTGA | 381 |
| | | Bord-2774 | 2755-2774 | ACTAGATCCCCCTGAAGGGT | 382 |
| | | Bord-2830 | 2810-2830 | AAGCGCAGTAATGCGTTAAGC | 383 |
| | Borrelia | Borr-435430 | 435406-435430 | ACCTGGTATTCTTCCAGGACCTTA | 384 |
| | | Borr-435441 | 435414-435441 | TTCTTCCAGGACCCTTAAAGGATATCTC | 385 |
| | | Borr-435519 | 435491-435519 | GAACGTAGCTACCCAGCACTTACCCTTGG | 386 |
| | | Borr-435562 | 435543-435562 | TTCGTCCATCTCGGTCCTCT | 387 |
| | | Borr-435574 | 435549-435574 | CATCTCGGTCCTCTCGTACTAAAGAT | 388 |
| | | Borr-435582 | 435558-435582 | CCTCTCGTACTAAAGATAGCTCCTC | 389 |
| | | Borr-435598 | 435576-435598 | GCTCCTCTCAAATATCCAACGCT | 390 |
| | | Borr-435613 | 435584-435613 | CAAATATCCAACGCTTGTGGCAGATAGGGA | 391 |
| | | Borr-435719 | 435699-435719 | TAGGATGCGATGAGCCGACAT | 392 |
| | | Borr-435744 | 435725-435744 | TGCCAAACCCTTCCGTCGAT | 393 |
| | | Borr-435812 | 435789-435812 | TTATTCGTTAAGTGACGGCGCTTC | 394 |
| | | Borr-435817 | 435795-435817 | GTTAAGTGACGGCGCTTCCACTT | 395 |
| | | Borr-435836 | 435816-435836 | TTGCCACCGCCAGATCACTAA | 396 |
| | | Borr-435844 | 435819-435844 | CCACCGCCAGATCACTAAGACCTACT | 397 |
| | | Borr-435873 | 435846-435873 | TCGTATCTGTTCGACTTGTCAGTCTTAC | 398 |
| | | Borr-435876 | 435856-435876 | TCGACTTGTCAGTCTTACAGT | 399 |
| | | Borr-435899 | 435880-435899 | GCTACCTTATGCCTTTACAC | 400 |
| | | Borr-435924 | 435902-435924 | ACAGAGTGATTTCCAACCACTCT | 401 |
| | | Borr-435928 | 435909-435928 | GATTTCCAACCACTCTAAGG | 402 |
| | | Borr-435934 | 435913-435934 | TCCAACCACTCTAAGGTAACCT | 403 |
| | | Borr-435945 | 435918-435945 | CCACTCTAAGGTAACCTTTGCGCACCTC | 404 |
| | | Borr-435950 | 435929-435950 | TAACCTTTGCGCACCTCCGTTA | 405 |
| | | Borr-435997 | 435970-435997 | CCCAGTCAAACTACCCACCTGGCACTCT | 406 |
| | | Borr-436009 | 435985-436009 | CACCTGGCACTCTCCTCATATTTCT | 407 |
| | | Borr-436070 | 436045-436070 | TCAAGATTGACTCCACTACCCCTGAC | 408 |
| | | Borr-435928 | 435909-435928 | GATTTCCAACCACTCTAAGG | 409 |
| | | Borr-436180 | 436157-436180 | CCGTCTAACCACAAGTAATCGGCA | 410 |
| | | Borr-436185 | 436163-436185 | AACCACAAGTAATCGGCATCTTC | 411 |
| | | Borr-436197 | 436168-436197 | CAAGTAATCGGCATCTTCACCGATACTTCA | 412 |
| | | Borr-436206 | 436187-436026 | CCGATACTTCAATTTCACCG | 413 |
| | | Borr-436219 | 436192-436219 | ACTTCAATTTCACCGAGCTCCACGTTGA | 414 |
| | | Borr-436247 | 436228-436247 | CCAAATCGTTACACCATTCG | 415 |
| | | Borr-436336 | 436317-436336 | CTGGGGCTTAAATTCAATGC | 416 |
| | | Borr-436403 | 436377-436403 | GGCAGGTGTCAGTCCCTATACTTCTCT | 417 |
| | | Borr-436428 | 436405-436428 | TACAGATTTGCAGAGACCTGTGTT | 418 |
| | | Borr-436452 | 436431-436452 | TGGTAAACAGTCGTTTGGACCA | 419 |
| | | Borr-436480 | 436460-436480 | GCTACCTAATTGCTTAGGTCG | 420 |
| | | Borr-436483 | 436464-436483 | CCTAATTGCTTAGGTCGTAC | 421 |
| | | Borr-436496 | 436470-436496 | TGCTTAGGTCGTACTTATCCCGAAGTT | 422 |
| | | Borr-436499 | 436479-436499 | CGTACTTATCCCGAAGTTACG | 423 |
| | | Borr-436507 | 436483-436507 | CTTATCCCGAAGTTACGTACGTATT | 424 |
| | | Borr-436530 | 436508-436530 | TTGCAGAGTTCCTTAACGTGGAT | 425 |
| | | Borr-436532 | 436513-436532 | GAGTTCCTTAACGTGGATTC | 426 |
| | | Borr-436534 | 436515-436534 | GTTCCTTAACGTGGATTCTC | 427 |
| | | Borr-436540 | 436517-436540 | TCCTTTAAGTGGATTCTCTCGCGC | 428 |
| | | Borr-436559 | 436529-436559 | ATTCTCTCGCGCGCCTTAGAATTTTCATCCC | 429 |
| | | Borr-436566 | 436547-436566 | GAATTTTCATCCCACCTACC | 430 |
| | | Borr-436572 | 436549-436572 | ATTTTCATCCCACCTACCTGTGTC | 431 |
| | | Borr-436579 | 436558-436579 | CCACCTACCTGTGTCGGTTTGC | 432 |
| | | Borr-436598 | 436574-436596 | GTTTGCGGTACGGTCCCTTATAG | 433 |
| | | Borr-436605 | 436580-436605 | GGTACGGTCCCTTATAGCCTAACCTT | 434 |
| | | Borr-436607 | 436588-436607 | CCCTTATAGCCTAACCTTAG | 435 |
| | | Borr-436626 | 436606-436626 | AGAAGCTATTTCTTGGGACCT | 436 |
| | | Borr-436630 | 436609-436630 | AGCTATTTCTTGGCACCTTGAC | 437 |
| | | Borr-436640 | 436614-436640 | TTTCTTGGCACCTTGACTACCTACATT | 438 |
| | | Borr-436689 | 436870-436639 | CATCTTAGCTCTCTTAACGG | 439 |
| | | Borr-436696 | 436677-436696 | GCTCTCTTAACGGATTTTCC | 440 |
| | | Borr-436746 | 436721-436746 | TAAACTAGGACAACCATCGCCTAGCA | 441 |
| | | Borr-436781 | 436756-436781 | CTCATGCGTCACTCCATCGAAACTAT | 442 |
| | | Borr-436793 | 436773-436793 | CGAAACTATAAGAGGTACGGG | 443 |
| | | Borr-436830 | 436308-436830 | TCCCATCGACTACACTTTTCAGC | 444 |
| | | Borr-436836 | 436817-436836 | CTACACTTTTCAGCTTTGCC | 445 |
| | | Borr-436844 | 436822-436844 | CTTTTCAGCTTTGCCTTAGGGGC | 446 |
| | | Borr-436851 | 436831-436851 | TTTGCCTTAGGGGCCGACTAA | 447 |
| | | Borr-436866 | 436846-436866 | GACTAACCCTGGGAAGACGAC | 448 |
| | | Borr-436915 | 436894-436915 | GCGAATGGGAATCTCACCCATT | 449 |
| | | Borr-436939 | 436920-436939 | CGTTACTCATACCTGCATTC | 450 |
| | | Borr-436944 | 436924-436944 | ACTCATACCTGCATTCTCACT | 451 |
| | | Borr-436946 | 436927-436946 | CATACCTGCATTCTCACTTC | 452 |
| | | Borr-436950 | 436929-436950 | TACCTGCATTCTCACTTCTGAT | 453 |
| | | Borr-436960 | 436937-436960 | TTCTCACTTCTGATACCTCCATCA | 454 |
| | | Borr-436970 | 436951-436970 | ACCTCCATCAAACTTTCCAG | 455 |
| | | Borr-436999 | 436975-436999 | ACTTCTCTGGCTTACAGAACGCTCC | 456 |
| | | Borr-437007 | 436984-437007 | GCTTACAGAACGCTCCCCTACCAT | 457 |
| | | Borr-437015 | 436990-437015 | AGAACGCTCCCCTACCATCTTAACTT | 458 |
| | | Borr-437018 | 436998-437018 | CCCCTACCATCTTAACTTTCG | 459 |
| | | Borr-437037 | 437017-437037 | CGTTAAGATCCAAAGCTTCGG | 460 |
| | | Borr-437045 | 437025-437045 | TCCAAAGCTTCGGTAATGTGT | 461 |
| | | Borr-437056 | 437031-437056 | GCTTCGGTAATGTGTTTAGCCCCGTT | 462 |
| | | Borr-437061 | 437039-437361 | AATGTGTTTAGCCCCGTTACATT | 463 |
| | | Borr-437067 | 437046-437067 | TTAGCCCCGTTACATTATCGGC | 464 |
| | | Borr-437072 | 437051-437072 | CCCGTTACATTATCGGCGCTTA | 465 |
| | | Borr-437077 | 437058-437077 | CATTATCGGCGCTTAAGTAC | 466 |
| | | Borr-437082 | 437060-437082 | TTATCGGCGCTTAAGTACTCGAC | 467 |
| | | Borr-437157 | 437137-437157 | GGCTGTTTACGTACCTAAACC | 468 |
| | | Borr-437160 | 437141-437160 | GTTTACGTACCTAAACCTCC | 469 |
| | | Borr-437166 | 437146-437166 | CGTACCTAAACCTCCTTTTCC | 470 |
| | | Borr-437169 | 437149-437169 | ACCTAAACCTCCTTTTCCACT | 471 |
| | | Borr-437175 | 437156-437175 | CCTCCTTTTCCACTTAACAC | 472 |
| | | Borr-437239 | 437215-437239 | CTCGACTATGAACCTTATCGCCCAT | 473 |
| | | Borr-437286 | 437267-437286 | GCATTCGGAGTTTAACTGAG | 474 |
| | | Borr-437291 | 437272-437291 | CGGAGTTTAACTGAGTTTGG | 475 |
| | | Borr-437307 | 437281-437307 | ACTGAGTTTGGTACCCTTTGACAGGCC | 476 |
| | | Borr-437311 | 437291-437311 | GTACCCTTTGACAGGCCCTAG | 477 |
| | | Borr-437331 | 437310-437331 | AGCTCAATTAGTGCTCTACCTC | 478 |
| | | Borr-437374 | 437355-437374 | CTTAAATCCATTTCGGGGAG | 479 |
| | | Borr-437380 | 437358-437380 | AAATCCATTTCGGGGAGAACCAG | 480 |
| | | Borr-437421 | 437399-437421 | AGCCTTTCACTCCTATTCACAGC | 481 |
| | | Borr-437445 | 437426-437445 | CCCTGCCTTTTTAAACAGAC | 482 |
| | | Borr-437459 | 437437-437459 | TAAACAGACTAGAGTTCGGCCCT | 483 |
| | | Borr-437465 | 437446-437465 | TAGAGTTCGGCCCTCCACTT | 484 |
| | | Borr-437480 | 437451-437480 | TTCGGCCCTCCACTTGGTTTACCCAAGCT | 485 |
| | | Borr-437489 | 437465-437489 | TGGTTTTACCCAAGCTTCAGCCTGG | 486 |
| | | Borr-437504 | 437475-437504 | CAAGCTTCAGCCTGGCCATAAATAGATCAC | 487 |
| | | Borr-437509 | 437488-437509 | GGCCATAAATAGATCACTCGGC | 488 |
| | | Borr-437516 | 437494-437516 | AAATAGATCACTCGGCTTCGGGT | 489 |
| | | Borr-437519 | 437500-437519 | ATCACTCGGCTTCGGGTCTA | 490 |
| | | Borr-437532 | 437508-437532 | GCTTCGGGTCTACCACATCTAACTA | 491 |
| | | Borr-437540 | 437520-437540 | CCACATCTAACTAAATCGCCC | 492 |
| | | Borr-437559 | 437533-437559 | AATCGCCCTTTTAAGACTCGCTTTCGC | 493 |
| | | Borr-437569 | 437543-437569 | TTAAGACTCGCTTTCGCTTCGACTCCA | 494 |
| | Brucella | Bruc-1107227 | 1107252-1107227 | GGGCAACCCACCTTAGATGACTAGAA | 495 |
| | | Bruc-1107198 | 1107224-1107198 | TCTGTTTTGTTGGAGCAACGCTGGATG | 496 |
| | | Bruc-1107188 | 1107212-1107188 | AGCAACGCTGGATGGGTTTACACC | 497 |
| | | Bruc-1107171 | 1107190-1107171 | ACCCATACAGACCGCTAGGT | 498 |
| | | Bruc-1107113 | 1107140-1107113 | CGCCAGCATTCCTCTGGAATGCGTACGG | 499 |
| | | Bruc-1106929 | 1106551-1106929 | CAGGCCAGTGGCTTTTGTGAATA | 500 |
| | | Bruc-1106664 | 1106686-1106664 | CCAAGGTTTGTCCTGGGTGACAG | 501 |
| | | Bruc-1106660 | 1106680-1106660 | TTTGTCCTGGGTGACAGCGTA | 502 |
| | | Bruc-1106284 | 1106309-1106284 | TCCGAATACCGGGGAGTACTAATCGG | 503 |
| | | Bruc-1106263 | 1106229-1106263 | GGGGAGTACTAATCGGCAGACACACGG | 504 |
| | | Bruc-1105786 | 1105805-1105786 | ACGGATCGCGTGTGTTGTGA | 505 |
| | | Bruc-1105778 | 1105802-1105778 | GATCGCGTGTGTTGTGAGGTCTTAT | 506 |
| | | Bruc-1105775 | 1105794-1105775 | GTGTTGTGAGGTCTTATTGG | 507 |
| | | Bruc-1105606 | 1105630-1105606 | GGAAGAAGCGTGACCTCACTATGGG | 508 |
| | | Bruc-1105594 | 1105622-1105594 | CGTGACCTCACTATGGGCAACCATAGGGG | 509 |
| | | Bruc-1105144 | 1105165-1105144 | TTATGGATGTCTAACTGCGGCC | 510 |
| | Burkholderia | Bur-322 | 300-322 | TGGAAAGTGCGGCCATAGCAGGT | 511 |
| | | Bur-326 | 307-326 | TGCGGCCATAGCAGGTGATA | 512 |
| | | Bur-335 | 312-335 | CCATAGCAGGTGATAGCCCTGTAG | 513 |
| | | Bur-362 | 343-362 | CAGTATGAAAGAACTAGGTG | 514 |
| | | Bur-667 | 648-667 | TTCAGTTGCTGGGCGTAGAC | 515 |
| | | Bur-731 | 707-731 | CGGTAACACGTACTGGAGGTCCGAA | 516 |
| | | Bur-746 | 723-746 | AGGTCCGAACCCACTAACGTTGAA | 517 |
| | | Bur-1162 | 1142-1162 | ATATACCGAAGCTGCGGATGC | 518 |
| | | Bur-1385 | 1362-1385 | GTAGCTGATGGGAAGCAGGTCAAT | 519 |
| | | Bur-1390 | 1371-1390 | GGGAAGCAGGTCAATATTCC | 520 |
| | | Bur-1744 | 1724-1744 | AGGGTGAAGGGGTTGCAATAA | 521 |
| | | Bur-1753 | 1732-1753 | GGGGTTGCAATAAACTGGTGGC | 522 |
| | | Bur-2118 | 2098-2118 | TGGGAGGCTATGAAACCGGAA | 523 |
| | | Bur-2269 | 2247-2269 | GTAACGGAGGAGTACGAAGGTAC | 524 |
| | | Bur-2279 | 2253-2279 | GAGGAGTACGAAGGTACGCTAGGTACG | 525 |
| | | Bur-2802 | 2780-2802 | CCTTGAAGGGTCGTTCGAGACCA | 526 |
| | | Bur-2843 | 2824-2843 | GTAAGCGCAGTAATGCGTTC | 527 |
| | Camphylobacter | Camp-183 | 160-183 | GAGCGAACGAGGGGAATTGAAACA | 528 |
| | | Camp-186 | 166-186 | ACGAGGGGAATTGAAACATCT | 529 |
| | | Camp-204 | 180-204 | AACATCTTAGTACCCTCAGGAAAAG | 530 |
| | | Camp-266 | 246-266 | AAGAGGGCAAACCCAGTGCTT | 531 |
| | | Camp-273 | 252-273 | GCAAACCCAGTGCTTGCACTGG | 532 |
| | | Camp-284 | 262-284 | TGCTTGCACTGGGGGTTGTAGGA | 533 |
| | | Camp-449 | 425-449 | TCGACCACGATCCAACCCTAAATAC | 534 |
| | | Camp-475 | 452-475 | ATACCAGATCGATAGTGCACAAGT | 535 |
| | | Camp-484 | 458-484 | GATCGATAGTGCACAAGTACCGTGAGG | 536 |
| | | Camp-508 | 488-508 | AGGTGAAAAGAACTGAGGTGA | 537 |
| | | Camp-519 | 495-519 | AAGAACTGAGGTGATCAGAGTGAAA | 538 |
| | | Camp-634 | 606-634 | CGAGTTGTGGTGTCTGGCAAGGTTAAGCA | 539 |
| | | Camp-702 | 682-702 | ATGCTGCAGACCCGAAACGAA | 540 |
| | | Camp-713 | 685-713 | CTGCAGACCCGAAACGAAGTGATCTATCC | 541 |
| | | Camp-722 | 699-722 | CGAAGTGATCTATCCATGAGCAAG | 542 |
| | | Camp-735 | 710-735 | ATCCAGCAGCAAGTTGAAGCTAGTGT | 543 |
| | | Camp-747 | 728-747 | GCTAGTGTAAGAACTAGTGG | 544 |
| | | Camp-825 | 804-825 | AAGGCCAATCAAACTTCGTGAT | 545 |
| | | Camp-832 | 812-832 | TCAAACTTCGTGATAGCTGGT | 546 |
| | | Camp-871 | 849-871 | TTAGGTATAGCGTTGTGTCGTAA | 547 |
| | | Camp-882 | 862-882 | TGTGTCGTAATATAAGGGGGT | 548 |
| | | Camp-929 | 906-929 | TACACCAATGTACCAAACCCTATC | 549 |
| | | Camp-970 | 948-970 | ATGTAATCACAGCAGTCAGGCGG | 550 |
| | | Camp-1138 | 1115-1138 | AATAGCTCACTGGTCTAGTGATTT | 551 |
| | | Camp-1148 | 1122-1148 | CACTGGTCTAGTGATTTTGCGCGGAAA | 552 |
| | | Camp-1162 | 1138-1162 | TTGCGCGGAAAATATAACGGGGCTA | 553 |
| | | Camp-1463 | 1443-1463 | TGTGAGAATCATTAACGCCGT | 554 |
| | | Camp-1553 | 1527-1553 | GAAAGGGGTAGACGATGGCAAATTGGT | 555 |
| | | Camp-1786 | 1759-1786 | TTAGCTAATGTTGCCCGTACCGTAAACC | 556 |
| | | Camp-1838 | 1817-1338 | GCGCGTGGAAGAACTCTCTTTA | 557 |
| | | Camp-1849 | 1830-1349 | CTCTCTTTAAGGAACTCTGC | 558 |
| | | Camp-1889 | 1863-1889 | ATCTTCGGTATAAGGTGTGGTTCGCTT | 559 |
| | | Camp-1946 | 1921-1946 | CTTACAACAAAGAGTCCCTCCCGACT | 560 |
| | | Camp-1972 | 1951-1972 | ACCAAAAACACAGCACTCTGCT | 561 |
| | | Camp-1997 | 1975-1997 | CTCGTAAGAGGATGTATAGGGTG | 562 |
| | | Camp-2006 | 1980-2006 | AAGAGGATGTATAGGGTGTGACGCCTG | 563 |
| | | Camp-2035 | 2013-2035 | GCTCGAAGGTTAATTGATGGGGT | 564 |
| | | Camp-2053 | 2026-2053 | TTGATGGGGTTAGCATTAGCGAAGCTCT | 565 |
| | | Camp-2075 | 2050-2075 | CTCTTGATCGAAGCCCGAGTAAACGG | 566 |
| | | Camp-2174 | 2145-2174 | CGTAACGAGATGGGAGCTGTCTCAAAGAGG | 567 |
| | | Camp-2186 | 2157-2186 | GGAGCTGTCTCAAAGAGGGATCCAGTGAAA | 568 |
| | | Camp-2192 | 2170-2192 | AGAGGGATCCAGTGAAATTGTAG | 569 |
| | | Camp-2262 | 2243-2262 | CCTTTACTACAGCTTGACAC | 570 |
| | | Camp-2268 | 2246-2268 | TTACTACAGCTTGACACTGCTAT | 571 |
| | | Camp-2273 | 2252-2273 | CAGCTTGACACTGCTATTTGGA | 572 |
| | | Camp-2298 | 2275-2298 | AAGAATGTGCAGGATAGGTGGGAG | 573 |
| | | Camp-2308 | 2284-2308 | CAGGATAGGTGGGAGGCTTTGAGTA | 574 |
| | | Camp-2350 | 2327-2350 | TGAGCCATTGTTGAGATACCACTC | 575 |
| | | Camp-2391 | 2369-2391 | AACCAGCTTGAGTTATCCTCAAG | 576 |
| | | Camp-2399 | 2374-2399 | GCTTGAGTTATCCTCAAGTGGGACAA | 577 |
| | | Camp-2409 | 2382-2409 | TATCCTCAAGTGGGACAATGTCTGGTGG | 578 |
| | | Camp-2453 | 2424-2453 | GCGGTCGCCTCCCAAATAATAACGGAGGCT | 579 |
| | | Camp-2471 | 2442-2471 | ATAACGGAGGCTTACAAAGGTTGGCTCAGA | 580 |
| | | Camp-2478 | 2454-2478 | TACAAAGGTTGGCTCAGAACGGTTG | 581 |
| | | Camp-2528 | 2505-2528 | TAAGCCAGCTTAACTGCAAGACAT | 582 |
| | | Camp-2539 | 2520-2539 | GCAAGACATACAAGTCAAGC | 583 |
| | | Camp-2580 | 2553-2580 | GGTCTTAGTGATCCGGTGGTTCTGTGTG | 584 |
| | | Camp-2804 | 2784-2804 | TATCTGCCGTGGGCGTAAGAA | 585 |
| | | Camp-2314 | 2788-2814 | TGCCGTGGGCGTAAGAAGATTGAAGAG | 586 |
| | | Camp-2837 | 2811-2837 | AGAGATTTGACCCTAGTACGAGAGGAC | 587 |
| | Chlamydia | Chl-856035 | 856016-856035 | ATAAGCAAAGACCCGGAGGT | 588 |
| | | Chl-856066 | 856040-856066 | GAATGGGGAAACCCGGTAGAGTAATAG | 589 |
| | | Chl-856164 | 856145-856164 | GAAATCGAAGAGATTCCCTG | 590 |
| | | Chl-856172 | 856147-856172 | AATCGAAGAGATTCCCTGTGTAGCGG | 591 |
| | | Chl-856267 | 856247-856267 | TCCTAGTTGAACACATCTGGA | 592 |
| | | Chl-856333 | 856313-856333 | AAGACCGACCTCAACACCTGA | 593 |
| | | Chl-856414 | 856395-856414 | CTAGTCAATGACCGATAGTG | 594 |
| | | Chl-856427 | 856398-856427 | GTCAATGACCGATAGTGAACCAGTACTGTG | 595 |
| | | Chl-856454 | 856427-856454 | GAAGGAAAGGCGAAAAGAACCCTTGTTA | 596 |
| | | Chl-856653 | 856633-856653 | AGTCGTTTGGTTTAGACACGA | 597 |
| | | Chl-856660 | 856641-856660 | GGTTTAGACACGAAACGAAG | 598 |
| | | Chl-856695 | 856670-856695 | TATGACCAGGTTGAAGCATGGGTAAA | 599 |
| | | Chl-856697 | 856678-856697 | GGTTGAAGCATGGGTAAAAC | 600 |
| | | Chl-856704 | 856685-856704 | GCATGGGTAAAACTATGTGG | 601 |
| | | Chl-856714 | 856688-856714 | TGGGTAAAACTATGTGGAGGACCGAAC | 602 |
| | | Chl-856829 | 856806-856829 | TAGGGTTAGCCTCGGATAATAAGC | 603 |
| | | Chl-856838 | 856818-856838 | CGGATAATAAGCTTTTGGGGG | 604 |
| | | Chl-856924 | 856904-856924 | AGCGAGTCCGGGAGATAGACA | 605 |
| | | Chl-856887 | 856968-856987 | GCCGATTAAGGTCCCTAATT | 606 |
| | | Chl-857104 | 857077-857104 | CACCAATCGAGAATCATTGCGCCGATAA | 607 |
| | | Chl-857112 | 857091-857112 | CATTGCGCCGATAATAAACGGG | 608 |
| | | Chl-857205 | 851184-857205 | AAGGTGTACCGGAAGGAGCGCT | 609 |
| | | Chl-857357 | 857331-857357 | AGGCGTAACTGCGTAGACGATGGAGCA | 610 |
| | | Chl-857394 | 857375-857394 | GCACCACCTAAAACTATAGC | 611 |
| | | Chl-857368 | 857344-857368 | TAGACGATGGAGCAGCAGGTTAAAT | 612 |
| | | Chl-857394 | 857369-857394 | ATTCCTGCACCACCTAAAACTATAGC | 613 |
| | | Chl-857458 | 857438-857458 | GTCCGTAGAGCGATGAGAACG | 614 |
| | | Chl-857478 | 857454-857478 | GAACGGTTAGTAGGCAAATCCGCTA | 615 |
| | | Chl-857495 | 857473-857495 | CCGCTAACATAAGATCAGGTCGC | 616 |
| | | Chl-857508 | 857438-857508 | AGGTCGCGATCAAGGGGAAT | 617 |
| | | Chl-857514 | 857491-857514 | GTCGCGATCAAGGGGAATCTTCGG | 618 |
| | | Chl-857536 | 857517-857536 | GAACCGATCGTGTGGAGCGA | 619 |
| | | Chl-857542 | 857523-857542 | ATGGTGTGGAGCGAGGCTTT | 620 |
| | | Chl-857550 | 857526-857550 | GTGTGGAGCGAGGTTTTCAAGAAAT | 621 |
| | | Chl-857580 | 857559-857580 | GCTGTTGATGGTGACCGTACCA | 622 |
| | | Chl-858146 | 858127-858146 | GGGAGTCAACGTTGAAATAC | 623 |
| | | Chl-858168 | 858144-858168 | TACTGGTCTTAACAAGCTGGGAATC | 624 |
| | | Chl-858199 | 858178-858199 | TCCATGAATCTGGAAAGATGGAC | 625 |
| | | Chl-858209 | 858188-858209 | TGGAAGATGGACATTGCCAGAC | 626 |
| | | Chl-858610 | 858591-858610 | TTCGTGGGCGCAGGATACTT | 627 |
| | | Chl-858680 | 858656-858680 | ATGGTGTGTCGGTTGTTTTGCCAAG | 628 |
| | | Chl-858775 | 858754-858775 | AAGGTATCCCAATGAGACTCCA | 629 |
| | | Chl-858782 | 858754-858782 | AAGGTATCCCAATGAGACTCCATGTAGAC | 630 |
| | Citrobacter | Cit-186 | 164-186 | CATAGCGTAATGAAGCGAACCGG | 631 |
| | | Cit-191 | 171-191 | TAATGAAGCGAACCGGGGGAA | 632 |
| | | Cit-356 | 332-356 | AGTCCCGTACACAAAAATGCACAGG | 633 |
| | | Cit-1021 | 992-1021 | GCCAGCTAAGGTCCCAAAGTCACAGTTAAG | 634 |
| | | Cit-1025 | 1004-1025 | CCCAAAGTCACAGTTAAGTGGG | 635 |
| | | Cit-1033 | 1010-1033 | GTCACAGTTAAGTGGGAAACGATG | 636 |
| | | Cit-1151 | 1122-1151 | CGGAAGATGTAACGGGGCTAAACTGTGCAC | 637 |
| | | Cit-1160 | 1136-1160 | GGGCTAAACTGTGCACCGAAGCTGC | 638 |
| | | Cit-1182 | 1158-1182 | TGCGGCAGCGACACTTATGTGTTGT | 639 |
| | | Cit-1186 | 1165-1186 | GCGACACTTATGTGTTGTTGGG | 640 |
| | | Cit-1194 | 1171-1194 | CTTATGTGTTGTTGGGTAGGGGAG | 641 |
| | | Cit-1519 | 1495-1519 | ATCCGGTACCTTTTTAACGCTGAGG | 642 |
| | | Cit-1523 | 1502-1523 | ACCTTTTTAACGCTGAGGTG | 643 |
| | | Cit-1736 | 1717-1736 | GTAGGTGAAGTGGTTTACTC | 644 |
| | | Cit-1740 | 1720-1740 | GGTGAAGTGGTTTACTCATGG | 645 |
| | | Cit-1747 | 1723-1747 | GAAGTGGTTTACTCATGGAGCTGAA | 646 |
| | | Cit-1759 | 1733-1759 | ACTCATGGAGCTGAAATCAGTCGAAGA | 647 |
| | | Cit-1878 | 1858-1878 | TGATGGGGTTATCGTAAGAGA | 648 |
| | | Cit-1883 | 1861-1883 | TGGGGTTATCGTAAGAGAAGCTC | 649 |
| | | Cit-1890 | 1870-1890 | CGTAAGAGAAGCTCTTGATCG | 650 |
| | | Cit-1897 | 1874-1897 | AGAGAAGCTCTTGATCGAAGCCCC | 651 |
| | | Cit-2210 | 2184-2210 | TAATGGCTGGTGTTCTAACGTGCACCC | 652 |
| | | Cit-2216 | 2193-2216 | GTGTTCTAACGTGGACCCGTTACC | 653 |
| | Clostridium | Clo-643 | 624-643 | CGGTATGTAGCAAGGTTAAG | 654 |
| | | Clo-814 | 793-814 | CATGGGATGAGCTGTGGATAGC | 655 |
| | | Clo-827 | 798-827 | GATGAGCTGTGGATAGCGGAGAAATTCCAA | 656 |
| | | Clo-1087 | 1068-1087 | GGATGTGGGATTTCTAAGAC | 657 |
| | | Clo-1104 | 1083-1104 | AAGACAACTAGGATGTTGGCTT | 658 |
| | | Clo-1163 | 1137-1163 | AGCTCACTAGTCAAGAGATCCTGCGCC | 659 |
| | | Clo-1435 | 1414-1435 | GGACAATCGGTTGATATTCCGA | 660 |
| | | Clo-1439 | 1420-1439 | TCGGTTGATATTCCGATACC | 661 |
| | | Clo-1665 | 1643-1665 | GCAAACCGACACAGGTAGGTGAG | 662 |
| | | Clo-1986 | 1965-1986 | ACCCGCACGAATGGCGTAATGA | 663 |
| | | Clo-2075 | 2054-2075 | ATTGGACGGAAAGACCCCGTAG | 664 |
| | | Clo-2090 | 2061-2090 | GGAAAGACCCCGTAGAGCTTTACTGTAGCT | 665 |
| | | Clo-2094 | 2075-2094 | GAGCTTTACTGTAGCTTAGC | 666 |
| | | Clo-2123 | 2102-2123 | TTCGGTATTGTCTGTACAGGAT | 667 |
| | | Clo-2411 | 2388-2411 | GGCTTAGTGATCCGGTGGTACCTC | 668 |
| | | Clo-2474 | 2446-2474 | CTCGGGGATAACAGGCTGATCTCCCCCAA | 669 |
| | Coryne- | Cor-934243 | 934223-934243 | AGCGGCATGTCGCGAGGTTAA | 670 |
| | bacterium | Cor-934344 | 934319-934344 | GTGATCTACCCATGGCCAGTGTGAAG | 671 |
| | | Cor-934388 | 934367-934388 | CGCGAACCCACTTAGGTTGAAA | 672 |
| | | Cor-934800 | 934781-934800 | AAGTACACCGCCGAAGCCGC | 673 |
| | | Cor-934892 | 934871-934892 | TGTGGAGTGTGTGCGAGTGAGA | 674 |
| | | Cor-934899 | 934873-934899 | TGGAGTGTGTGCGAGTGAGAATGCAGG | 675 |
| | | Cor-934911 | 934882-934911 | TGCGAGTGAGAATCGAGGCATGAGTAACGA | 676 |
| | | Cor-934951 | 934931-934951 | TCCGCCGGATGACTAAGGGTT | 677 |
| | | Cor-934985 | 934964-934985 | TAATCTTCCCAGGGTGAGTCGG | 678 |
| | | Cor-935215 | 935194-935215 | TGGATTGTGGTGTAAGCGTGTG | 679 |
| | | Cor-935308 | 935289-935308 | GTGATCCTGTACTGTCGAGA | 680 |
| | | Cor-935322 | 935293-935322 | TCCTGTACTGTCGAGAAAAGCCTCTAGCGA | 681 |
| | | Cor-935505 | 935485-935505 | GGTCGCAGAGAATAGAGGGAA | 682 |
| | | Cor-935574 | 935550-935574 | GTTGATGTATACGGACTGACGCCTG | 683 |
| | | Cor-935735 | 935708-935735 | CTGCACGAATGGCGTAACGACTTCCCTG | 684 |
| | | Cor-935749 | 935720-935749 | CGTAACGACTTCCCTGCTGTCTCAACCACA | 685 |
| | | Cor-935766 | 935743-935766 | AACCACAGGCCCGGTGAAATTGCA | 686 |
| | | Cor-935774 | 935749-935774 | AGGCCCGGTGAAATTGCAGTACGAGT | 687 |
| | | Cor-935827 | 935308-935827 | ACCCCGGGACCTTCACTATA | 688 |
| | | Cor-935834 | 935811-935834 | CCGGGACCTTCACTATAGCTTGGT | 689 |
| | | Cor-935869 | 935847-935869 | TTCGGTTTGTGTAGGATAGGTGG | 690 |
| | | Cor-935935 | 935918-935935 | GAAATACCACTCTGATCGGATTG | 691 |
| | | Cor-935968 | 935947-935968 | CTTGGCCCATGATCTGGGTTGG | 692 |
| | | Cor-935975 | 935954-935975 | CATGATCTGGGTTGGGGACAGT | 693 |
| | | Cor-936080 | 936059-936080 | CAGGTGGTGAGTGTAAGTGCAC | 694 |
| | | Cor-936155 | 936130-936155 | GACTAGTGATCCGGCACCTACTTGTG | 695 |
| | | Cor-936407 | 936386-936407 | GAAGGCTGTCCCTAGTACGAGA | 696 |
| | | Cor-936518 | 933496-936518 | GCATCTAAGCGGGAAGCCTGTTT | 697 |
| | Enterobacter | Entb-1808 | 1789-1808 | TGCGGGTGGAGCTGAAATCA | 698 |
| | Enterococcus | Entc-310 | 291-310 | TTGGGGTTGTAGGACTCCRA | 699 |
| | | Entc-414 | 393-414 | CACCTAGGAGGATCCTGAGTAC | 700 |
| | | Entc-907 | 887-907 | AGAATGATGGAGGTAGAGCAC | 701 |
| | | Entc-2219 | 2198-2219 | GTTATGVCCACTCTAACCCGC | 702 |
| | | Entc-2838 | 2817-2838 | GTAAGATCCCTGAGAGATGATC | 703 |
| | Fusobacterium | Fuso-72 | 47-72 | AGCCGATGAAGGACGTGGTAAGCTGC | 704 |
| | | Fuso-203 | 179-203 | AACATCTAAGTAACGCGAGGAAAAG | 705 |
| | | Fuso-207 | 188-207 | GTAACGCGAGGAAAAAGAAAG | 706 |
| | | Fuso-451 | 425-451 | CTCCCAAGTAACATGGAACACGAGGAA | 707 |
| | | Fuso-464 | 435-464 | ACATGGAACACGAGGAATTCTGTGTGAATC | 708 |
| | | Fuso-1198 | 1175-1198 | CCGACAATGTAACGGGGCTAAGTT | 709 |
| | | Fuso-1316 | 1297-1316 | GAATGCAGGAATAAGTAGCG | 710 |
| | | Fuso-1373 | 1350-1373 | CCAAGGTTTTCAGGGTAAAGCTTG | 711 |
| | | Fuso-1400 | 1380-1400 | CTGAGTAAGCCGGGACCTAAG | 712 |
| | | Fuso-1682 | 1656-1682 | GACACAGGTGGTCAGGATGAGAAATCT | 713 |
| | | Fuso-1171 | 1687-1713 | CGGACAGGCTAACTCTCGTTAAGGAAC | 714 |
| | | Fuso-1720 | 1698-1720 | ACTCTCGTTAAGGAACTCTGCAA | 715 |
| | | Fuso-1812 | 1792-1812 | GTCGCAGTGAAGAGGCTCAAG | 716 |
| | | Fuso-1824 | 1795-1824 | GCAGTGAAGAGGCTCAAGCAACTGTTTAAC | 717 |
| | | Fuso-2025 | 2004-2025 | TGGTGTAATGATTTGAGCGCTG | 718 |
| | | Fuso-2034 | 2011-2084 | ATGATTTGAGCGCTGTCTTGACGG | 719 |
| | | Fuso-2050 | 2031-2050 | ACGGGAGGCCTGGTGAAATT | 720 |
| | | Fuso-2140 | 2120-2140 | GGTATTGGGTTTTGGCATTGC | 721 |
| | | Fuso-2145 | 2124-2145 | TTGGGTTTTGGCATTGCATGTA | 722 |
| | | Fuso-2158 | 2139-2158 | GCATGTATAGGATAGTTGGG | 723 |
| | | Fuso-2217 | 2196-2217 | TCGGTGGAATACCAACCATTCA | 724 |
| | | Fuso-2499 | 2471-2499 | CCTGGGGATAACAGGCTGATCCTACCCGA | 725 |
| | Haemophilus | Has-14505 | 14486-14505 | TGAACCGGTCGAAGATACCA | 726 |
| | | Has-14732 | 14707-14732 | GCACGAATGGCATAATGATGGCCAGG | 727 |
| | | Has-14738 | 14717-14738 | CATAATGATGGCCAGGCTGTCT | 728 |
| | | Has-15050 | 15021-15050 | AGGAGCACGAAGGTTTGCTAATCACGGTCG | 729 |
| | | Has-15055 | 15033-15055 | GTTTGCTAATCACGGTCGGACAT | 730 |
| | Helicobacter | Heli-378 | 362-378 | GGGGTTGAGGACTGCAA | 731 |
| | | Heli-609 | 588-609 | AACCGCAGTGAGCGGAGTGAAA | 732 |
| | | Heli-619 | 591-619 | CGCAGTGAGCGGAGTGAAATAGAACCTGA | 733 |
| | | Heli-665 | 643-665 | GAGCCCTATGATTTATCAGGGTG | 734 |
| | | Heli-674 | 652-674 | GATTTATCAGGGTGATGGACTGC | 735 |
| | | Heli-787 | 766-787 | GTCAGATGCTGCAGACCCGAAG | 736 |
| | | Heli-860 | 836-860 | GAGGACTGAACTCGTACCCATTGAA | 737 |
| | | Heli-868 | 846-868 | CTCGTACCCATTGAAACGGGTTG | 738 |
| | | Heli-886 | 864-886 | GGTTGGGATGAGCTGTGGATAGG | 739 |
| | | Heli-906 | 879-906 | TGGATAGGGGTGAAAGGCCAAACAAACT | 740 |
| | | Heli-958 | 939-958 | AGGTATAGCCTCAAGTGATA | 741 |
| | | Heli-997 | 978-997 | TGATTGGGCTAGGGCTGCTC | 742 |
| | | Heli-1060 | 1039-1060 | CGTATCTTGGGAGTCAGGCGGT | 743 |
| | | Heli-1266 | 1241-1266 | ATAACGGGGCTAAGATAGACACCGAA | 744 |
| | | Heli-1344 | 1323-1344 | ATACCGGTAAGGAGTGCTGGAG | 745 |
| | | Heli-1430 | 1409-1430 | ATCTAAGGTTTCCTACGCGATG | 746 |
| | | Heli-1690 | 1673-1690 | CCGTCGTGCCAAGAAAAG | 747 |
| | | Heli-1749 | 1720-1749 | CGCAAACCGACACAGGTAGATGAGATGAGT | 748 |
| | | Heli-1761 | 1739-1761 | ATGAGATGAGTATTCTAAGGCGC | 749 |
| | | Heli-1856 | 1835-1856 | GGTCTCAGCAAAGAGTCCCTCC | 750 |
| | | Heli-2089 | 2067-2089 | CGAGATGGGAGCTGTCTCAACCA | 751 |
| | | Heli-2801 | 2781-2801 | AGTTGTTCTGCCAAGAGCATC | 752 |
| | | Heli-2920 | 2900-2920 | TGCTTGATAGGGTAGATGTGT | 753 |
| | Klebsiella | Kleb-93 | 74-93 | CGACACACAGCTAATGTGTG | 754 |
| | | Kleb-124 | 105-124 | CAAATTTTCGCGACACGACG | 755 |
| | | Kleb-134 | 108-134 | ATTTTCGCGACACGACGATGTTTTACG | 756 |
| | | Kleb-1891 | 1872-1891 | GTGAAGTGACTTGCTCATGG | 757 |
| | | Kleb-1898 | 1874-1898 | GAAGTGACTTGCTCATGGAGCTGAA | 758 |
| | | Kleb-1903 | 1884-1903 | GCTCATGGAGCTGAAATCAG | 759 |
| | Legionella | Legi-76 | 51-76 | ATACATAGGATGCAAAGGCGAACTCG | 760 |
| | | Legi-147 | 122-147 | AGAGAGATTCTCCAAGTAGCGGCGAG | 761 |
| | | Legi-178 | 156-178 | GAGGAGCCTGGCGTGATTTATTA | 762 |
| | | Legi-213 | 193-213 | ACAATTTGGGAAAGTTGGCGA | 763 |
| | | Legi-236 | 213-236 | ATAGAGGGTGAAAGCCCCGTATAC | 764 |
| | | Legi-248 | 219-248 | GGTGAAAGCCCGTATACGAAGGTTTGATG | 765 |
| | | Legi-255 | 235-255 | ACGAAGGTTTGATGAGGAACT | 766 |
| | | Legi-263 | 239-263 | AGGTTTGATGAGGAACTAGGCACGC | 767 |
| | | Legi-290 | 271-290 | TAGGCCGGGACACGTGAAAT | 768 |
| | | Legi-302 | 277-302 | GGGACACGTGAAATCCTGGTTGAAGA | 769 |
| | | Legi-328 | 306-328 | GTGGACCATCATCCAAGGCTAAA | 770 |
| | | Legi-350 | 331-350 | CTACTTACTGACCGATAGTG | 771 |
| | | Legi-423 | 403-423 | AGAATCTGAAACCGTTTGCGT | 772 |
| | | Legi-472 | 443-472 | GCTGTGTGACTGCGTACCTTTTGTA | 773 |
| | | Legi-520 | 499-520 | GGCGAGGTTAACTGAATAAGGG | 774 |
| | | Legi-571 | 550-571 | CGATAGTCGCTGGGAGTAGACC | 775 |
| | | Legi-593 | 574-593 | AAACCGGGCGATCTAGCCAT | 776 |
| | | Legi-650 | 626-650 | AGGTCCGAACCGGGTAATGTTGAAA | 777 |
| | | Legi-674 | 351-674 | AATTATCGGATGACGTGTGGCTAG | 778 |
| | | Legi-794 | 775-794 | TAGGGGGCTGTCATGGCTTA | 779 |
| | | Legi-842 | 817-842 | TACCGGCTAATTGAATCACGGGAGAC | 780 |
| | | Legi-847 | 825-847 | AATTGAATCACGGGAGACACACG | 781 |
| | | Legi-920 | 892-920 | CCAGCTAAGGTCCCCAAGTACTAGTTAAG | 782 |
| | | Legi-924 | 903-924 | CCCCAAGTACTAGTTAAGTGGG | 783 |
| | | Legi-1064 | 1045-1064 | TAGTCACCGAAGCTGCGGAT | 784 |
| | | Legi-1192 | 1167-1192 | CGATAATGTGGGTGAAAAGCCCACAC | 785 |
| | | Legi-1217 | 1197-1217 | GAAGTCCCAGGTTTCCTGCAC | 786 |
| | | Legi-1242 | 1218-1242 | GACGTTAATCGGAGCAGGGTGAGTC | 787 |
| | | Legi-1282 | 1256-1282 | AGGCTGAAGAGCGTAGTCGATGGGAAC | 788 |
| | | Legi-1329 | 1305-1329 | TTATAAGTGGTGAaaTGGGGACGAA | 789 |
| | | Legi-1337 | 1312-1337 | TGGTGAAGTGGGGACGAAGAAGGCTA | 790 |
| | | Legi-1390 | 1363-1390 | GTACTTGCATGTAGGGGGGAAGACTTGG | 791 |
| | | Legi-1417 | 1395-1417 | TCCGGTTTTCCATAACTCTGAGG | 792 |
| | | Legi-L471 | 1443-1471 | ACAGAGAAGTCATTGATGCCCGGCTTCCA | 793 |
| | | Legi-1492 | 1464-1492 | GGCTTCGAGGAAAAGCTGCTAGCCATAAC | 794 |
| | | Legi-1516 | 1491-1516 | ACTTATAGAGAACCGTACCGCAAACC | 795 |
| | | Legi-1531 | 1511-1531 | CAAACCGACACAGGTGGACAG | 796 |
| | | Legi-1632 | 1611-1632 | GCCCTTTCTGGTGATGGGATTT | 797 |
| | | Legi-1646 | 1626-1646 | GGGATTTACTTTCAGAGCTGG | 798 |
| | | Legi-1652 | 1631-1652 | TTACTTTCAGAGCTGGAGAGGG | 799 |
| | | Legi-1685 | 1663-1685 | CAGGTGGCTGCGACTGTTTATTA | 800 |
| | | Legi-1713 | 1689-1713 | ACACAGCACTCTCCAAATTCGTAAG | 801 |
| | | Legi-1728 | 1707-1728 | TCGTAAGAAGACGTATAGGGTG | 802 |
| | | Legi-1734 | 1711-1734 | AAGAAGAGGTATAGGGTGTGACGC | 803 |
| | | Legi-1784 | 1756-1784 | ATTGATGGGGTTATCTTCGGAGAAGCTCT | 804 |
| | | Legi-2014 | 1989-2014 | TGCACTGGACTTTGATGATGACTGTG | 805 |
| | | Legi-2030 | 2004-2030 | TGATGACTGTGTAGGATAGGTGGGAGG | 806 |
| | | Legi-2039 | 2018-2039 | GATAGGTGGGAGGCTGTGAAGT | 807 |
| | | Legi-2076 | 2054-2076 | TCATGGAGCCGCCCTTGAAATAC | 808 |
| | | Legi-2086 | 2063-2086 | CGCCCTTGAAATACCACCCTGTTG | 809 |
| | | Legi-2098 | 2075-2098 | ACCACCCTGTTGTTATTGAGGTTC | 810 |
| | | Legi-2117 | 2090-2117 | TTGAGGTTCTAACTTGGTCCAGTAATCC | 811 |
| | | Legi-2156 | 2134-2156 | ATGATGGGTAGTTTGACTGGGGC | 812 |
| | | Legi-2199 | 2179-2199 | GAGGAGCACAAAGGTACCCTC | 813 |
| | | Legi-2207 | 2183-2207 | AGCACAAAGGTACCCTCGGTACGGT | 814 |
| | | Legi-2222 | 2202-2222 | TACGGTCGGACATCGTACCAA | 815 |
| | | Legi-2229 | 2205-2229 | GGTCGGACATCGTACCAAGAGTGTA | 816 |
| | | Legi-2235 | 2214-2235 | TCGTACCAAGAGTGTAAAGGCA | 817 |
| | | Legi-2261 | 2241-2261 | GTGCTTGACTGCGAGAGTGAC | 818 |
| | | Legi-2289 | 2263-2289 | GCTCGAGCAGGAACGAAAGTTGGTCTT | 819 |
| | Listeia | Lis-53 | 34-53 | AGGAAGGCAGACCCAGGGAA | 820 |
| | | Lis-111 | 90-111 | AATCGATTTCCTGAGTAGCGGC | 821 |
| | | Lis-158 | 137-158 | AAGAAGCTTGCTTCTTGGGGTT | 822 |
| | | Lis-183 | 163-183 | GACACTCTATACGGAGTTACA | 823 |
| | | Lis-219 | 198-219 | ATGAAGCGGTCTGGAAAGGCCC | 824 |
| | | Lis-275 | 254-275 | CTTTCCCTCCAGAGTGGATCCT | 825 |
| | | Lis-442 | 414-442 | CCTGAAACCGTGTGCCTACAAGTAGTTAG | 826 |
| | | Lis-451 | 425-451 | GTGCCTACAAGTAGTTAGAGCCCGTTA | 827 |
| | | Lis-456 | 435-456 | GTAGTTAGAGCCCGTTAATGGG | 828 |
| | | Lis-538 | 515-538 | GGAAAAAGCGGAGCCGTAGCGAAA | 829 |
| | | Lis-587 | 561-587 | AAGTAACAGGTCGTAGACCCGAAACCA | 830 |
| | | Lis-755 | 733-755 | TTTAGGGCTAGCCTCGAGGTAAA | 831 |
| | | Lis-760 | 739-760 | GCTAGCCTCGAGGTAAAGAGTC | 832 |
| | | Lis-797 | 776-797 | TGTTTGGACTAGGGGCCCTTCT | 833 |
| | | Lis-817 | 791-817 | CCCTTCTCGGGTTACCGAATTCAGATA | 834 |
| | | Lis-1041 | 1021-1041 | GACCCCGCGCCGAAAATGTAC | 835 |
| | | Lis-1076 | 1056-1076 | TTACCGAAACTGTGGATGAAC | 836 |
| | | Lis-1108 | 1087-1108 | GTTCGTGGTAGGAGAGCGTTCT | 837 |
| | | Lis-1141 | 1122-1141 | TCAGACCGGAAGGACTGGTG | 838 |
| | | Lis-1215 | 1186-1215 | GTGAGAATCCCTTCCACCGAATATCTAAGG | 839 |
| | | Lis-1370 | 1352-1370 | GGAATCGCACGAATGGAAA | 840 |
| | | Lis-1392 | 1372-1392 | GTGCGTCCAAGCAGTGAGTGT | 841 |
| | | Lis-1438 | 1418-1438 | CGAAGCATGAGCTGTGATGGG | 842 |
| | | Lis-1472 | 1451-1472 | GTACGGAAGTTCCTGATTTCAC | 843 |
| | | Lis-1482 | 1453-1482 | ACGGAAGTTCCTGATTTCACGCTGTCAAGA | 844 |
| | | Lis-1493 | 1468-1493 | TTCACGCTGTCAAGAAAAGCCTCTAG | 845 |
| | | Lis-1523 | 1502-1523 | GTACTGCCCGTACCGCAAACCG | 846 |
| | | Lis-1630 | 1605-1630 | GGGAGAAGGGGTGCTCTATTAGGGTG | 847 |
| | | Lis-1983 | 1958-1983 | CCCGTGGAGCTTTACTGCAACCTGAT | 848 |
| | | Lis-1991 | 1972-1991 | CTGCAACCTGATATGGAATG | 849 |
| | | Lis-2007 | 1985-2007 | TGGAATGTTTGTACCGCTTGTAC | 850 |
| | | Lis-2069 | 2049-2069 | AGGAGGCAATGGTGGGATACT | 851 |
| | | Lis-2127 | 2106-2127 | AGCGCGTGGGGAGACAGTGTCA | 852 |
| | | Lis-2217 | 2196-2217 | TGGATGGAAATCATTCGCAGAG | 853 |
| | Morganella | Mor-143 | 124-143 | AGCACAGTGAGCGGAGCATA | 854 |
| | | Mor-386 | 367-386 | CGTTGCACTATCATTACCTG | 855 |
| | | Mor-413 | 391-413 | CATAGGGTAATGAAGCGAACCGG | 856 |
| | | Mor-420 | 396-420 | GGTAATGAAGCGAACCGGGAGAACT | 857 |
| | | Mor-473 | 447-473 | GAAATCAAACGAGATTCCCCCAGTAGC | 858 |
| | | Mor-535 | 516-535 | GTGTTAAGAGAACAGTCTGG | 859 |
| | | Mor-545 | 521-545 | AAGAGAACAGTCTGGAAAGGCTGGC | 860 |
| | | Mor-573 | 547-573 | ACAGCAGGTGATAGCCCTGTATCTGAA | 861 |
| | | Mor-584 | 556-584 | GATAGCCCTGTATCTGAAAGCACTGGTGT | 862 |
| | | Mor-593 | 567-533 | ATCTGAAAGCACTGGTGTTGTGAGTCC | 863 |
| | | Mor-620 | 598-620 | AGTAAGGCGGGACACGTGTTATC | 864 |
| | | Mor-1386 | 1357-1386 | AACGGGGCTAAATTATGCACCGAAGCCGCG | 865 |
| | | Mor-1452 | 1431-1452 | CCTGTGAAGGTGACCTGTGAGG | 866 |
| | | Mor-2089 | 2063-2089 | CCGGTGCCGGAAGGTTAATTGATGAGG | 867 |
| | | Mor-2100 | 2075-2100 | GGTTAATTGATGAGGTCAGCCGCAAG | 868 |
| | | Mor-2123 | 2095-2123 | CGCAAGGCGAAGCTTCTGATCGAAGCCCC | 869 |
| | | Mor-2130 | 2108-2130 | TTCTGATCGAAGCCCCGGTAAAC | 870 |
| | | Mor-2408 | 2379-2408 | TGCACGGAGCCATCCTTGAAATACCACCCT | 871 |
| | Mycobacteria | MB-862 | 833-862 | GCCAGGGTGAAGCGCGGGTAAGACCGCGTG | 872 |
| | | MB-872 | 845-872 | CGCGGGTAAGACCGCGTGGAGGCCCGAA | 873 |
| | | MB-1253 | 1234-1253 | TAGCTCACTGGTCAAGTGAT | 874 |
| | | MB-2047 | 2027-2047 | TCCGTGCGAAGTCGCAAGACG | 875 |
| | | MB-2094 | 2074-2094 | TGCTGGAAGGTTAAGAGGACC | 876 |
| | | MB-2134 | 2115-2134 | GCGGAGAATTTAAGCCCCAG | 877 |
| | | MB-2250 | 2229-2250 | GTCTCAACCATAGACTCGGCGA | 878 |
| | | MB-2543 | 2524-2543 | AAGGTTCCCTCAACCTGGWC | 879 |
| | | MB-3006 | 2987-3006 | AGCATCTAAGCGGGAAACCT | 880 |
| | Mycoplasma | MP-261 | 241-261 | TGTGTAGTGGCGAGCGAAAGC | 881 |
| | | MP-454 | 434-454 | GAATCTGCCCAGACCATTGGG | 882 |
| | | MP-522 | 502-522 | GGAAAGGTGAAAAGAACCCAG | 883 |
| | | MP-571 | 550-571 | CATATGCCTACAACGTGTCAGA | 884 |
| | | MP-1125 | 1097-1125 | CTTAGAAGCAGCCATCGTTTAAAGAGTGC | 885 |
| | | MP-1136 | 1108-1136 | CCATCGTTTAAAGAGTGCGTAACAGCTCA | 886 |
| | | MP-1146 | 1127-1146 | TAACAGCTCACTTGTCGAGT | 887 |
| | | MP-1150 | 1129-1150 | ACAGCTCACTTGTCGAGTGTTT | 888 |
| | | MP-1187 | 1171-1187 | GGCTAAGTATATTACCG | 889 |
| | | MP-1837 | 1818-1837 | TGATGTATATGGGGTGACAC | 890 |
| | | MB-2482 | 2460-2482 | GGCTGATACTGCCCAAGAGTTCA | 891 |
| | Neisseria | Nei-63204 | 63183-63204 | CTGAATAAGTGCATCAGGTGGA | 892 |
| | | Nei-63211 | 63187-63211 | ATAAGTGCATCAGGTGGATGCCTTG | 893 |
| | | Nei-63239 | 63218-63239 | ATAGGCGACGAAGGACGTGTAA | 894 |
| | | Nei-63244 | 63224-63244 | GACGAAGGACGTGTAAGCCTG | 895 |
| | | Nei-63278 | 63256-63278 | GGGGAGCTGGCAATAAAGCAATG | 896 |
| | | Nei-63291 | 63264-63291 | GGCAATAAAGCAATGATCCCGCGATGTC | 897 |
| | | Nei-63296 | 63275-63296 | AATGATCCCGCGATGTCCGAAT | 898 |
| | | Nei-63334 | 63315-63334 | CTGTGCAGTATCCTAAGTTG | 899 |
| | | Nei-63369 | 63341-63369 | TAGACTTAGAGAAGCGAACCCGGAGAACT | 900 |
| | | Nei-63381 | 63356-63381 | GAACCCGGAGAACTGAACCATCTAAG | 901 |
| | | Nei-63462 | 63435-63462 | CGGAGGAGCCTGTACGTAATAACTGTCG | 902 |
| | | Nei-63478 | 63459-63478 | GTCGAGATAGAAGAACAAGC | 903 |
| | | Nei-63483 | 63461-63483 | CGAGATAGAAGAACAAGCTGGGA | 904 |
| | | Nei-63516 | 63493-63516 | ATAGTGGGTGACAGTCCCGTATTC | 905 |
| | | Nei-63527 | 63499-63527 | GGTGACAGTCCCGTATTCGAAATCTCAAC | 906 |
| | | Nei-63536 | 63513-63536 | ATTCGAAATCTCAACAGCGGTACT | 907 |
| | | Nei-63557 | 63335-63557 | CTAAGCGTACGAAAAGTAGGGCG | 908 |
| | | Nei-63614 | 63594-63614 | TCCTCCAAGGCTAAATACTCA | 909 |
| | | Nei-63696 | 63677-63696 | GAAACAGAACCTGAAACCTG | 910 |
| | | Nei-63705 | 63680-63705 | ACAGAACCTGAAACCTGATGCATACA | 911 |
| | | Nei-63720 | 63693-63720 | CCTGATGCATACAAACAGTGGGAGCGCC | 912 |
| | | Nei-63748 | 63720-63743 | CCTAGTGGTGTGACTGCGTACCTTTTGTA | 913 |
| | | Nei-63773 | 63752-63773 | TGGGTCAACGACTTACATTCAG | 914 |
| | | Nei-63782 | 63759-63782 | ACGACTTACATTCAGTAGCGAGCT | 915 |
| | | Nei-63833 | 63804-63833 | GGGAAACCGAGTCTTAATAGGGCGATGAGT | 916 |
| | | Nei-63839 | 63816-43839 | CTTAATAGGGCGATGAGTTGCTGG | 917 |
| | | Nei-63848 | 63825-63848 | GCGATGAGTTGCTGGGTGTAGACC | 918 |
| | | Nei-63869 | 63840-63869 | GTGTAGACCCGAAACCGAGTGATCTATCCA | 919 |
| | | Nei-63901 | 63873-63901 | CCAGGTTGAAGGTGCCGTAACAGGTACTG | 920 |
| | | Nei-63911 | 63887-63911 | CCGTAACAGGTACTGGAGGACCGAA | 921 |
| | | Nei-64024 | 63398-64024 | ACTATTTAGGTAGTGCCTCGAGCAAGA | 922 |
| | | Nei-64328 | 64007-64028 | GTAGTGCCTCGAGCAAGACACT | 923 |
| | | Nei-64034 | 64011-64034 | TGCCTCGAGCAAGACACTGATGGG | 924 |
| | | Nei-64076 | 64055-64076 | AGGGGGTTATTGCAACTTACCA | 925 |
| | | Nei-64112 | 64093-64112 | GAATACCATCAAGTGGTTCC | 926 |
| | | Nei-64116 | 64095-64116 | ATACCATCAAGTGGTTCCTCGG | 927 |
| | | Nei-64122 | 64099-64122 | CATCAAGTGGTTCCTCGGGAGACA | 928 |
| | | Nei-64193 | 64174-64193 | GCTAAGGTCCCAAATGATAG | 929 |
| | | Nei-64223 | 64196-64223 | TAAGTGGTAAACGAAGTGGGAAGGCCCA | 930 |
| | | Nei-64327 | 64301-64327 | GGAAGATGTAACGGGGCTCAAATCTAT | 931 |
| | | Nei-64333 | 64311-64333 | ACGGGCTCAAATCTATAACCGA | 932 |
| | | Nei-64345 | 64321-64345 | AATCTATAACCGAAGCTGCGGATGC | 933 |
| | | Nei-64352 | 64333-64352 | AAGCTGCGGATGCCGGTTTA | 934 |
| | | Nei-64368 | 64349-64368 | TTTACCGGCATGGTAGGGGA | 935 |
| | | Nei-64436 | 64416-64436 | TCAGAAGTGCGAATGTTGACA | 936 |
| | | Nei-64445 | 64424-64445 | GCGAATGTTGACATGAGTAGCG | 937 |
| | | Nei-64493 | 64471-64493 | CCGAAAGCCCAAGGTTTCCTGCG | 938 |
| | | Nei-64704 | 64680-64704 | TCTTAACACCGAGAAGTGACGACGA | 939 |
| | | Nei-64727 | 64701-64727 | ACGAGTGTCTACGGACACGAAGCAACC | 940 |
| | | Nei-64743 | 64719-64743 | GAAGCAACCGATACCACGCTTCCAG | 941 |
| | | Nei-64788 | 64761-64788 | CAGTTTGAATCGAACCGTACCGCAAACC | 942 |
| | | Nei-64887 | 64868-64887 | TTCGGGAGAAGGTATGCCCT | 943 |
| | | Nei-64915 | 64888-64915 | CTAAGGTTAAGGACTTGCTCCGTAAGCC | 944 |
| | | Nei-64939 | 64919-64939 | GAGGGTCGCAGAGAATAGGTG | 945 |
| | | Nei-64943 | 64923-64943 | GTCGCAGAGAATAGGTGGCTG | 946 |
| | | Nei-65056 | 65028-65056 | TTGAAGATGTGAGAGCATCGGATCGAAGC | 947 |
| | | Nei-65068 | 65044-65068 | ATCGGATCGAAGCCCCAGTAAACGG | 948 |
| | | Nei-65204 | 65181-65204 | TGAAGTGGTTGTGAAGATGCAATC | 949 |
| | | Nei-65224 | 65205-65224 | TACCCGCTGCTAGACGGAAA | 950 |
| | | Nei-65276 | 65252-65276 | GCATTGGACTTTGAAGTCACTTGTG | 951 |
| | | Nei-65292 | 65266-65292 | AGTCACTTGTGTAGGATAGGTGGGAGG | 952 |
| | | Nei-65302 | 65295-65302 | TGTAGGATAGGTGGGAGGCTTAGAAGCA | 953 |
| | | Nei-65309 | 65285-65309 | GTGGGAGGCTTAGAAGCAGAGACGC | 954 |
| | | Nei-65317 | 65292-63317 | GCTTAGAAGCAGAGACGCCAGTCTCT | 955 |
| | | Nei-65362 | 65339-65362 | CACCCTGGTGTCTTTGAGGTTCTA | 956 |
| | | Nei-65374 | 65345-65374 | GGTGTCTTTGAGGTTCTAACCCAGACCCGT | 957 |
| | | Nei-65378 | 65357-65378 | GTTCTAACCCAGACCCGTCATC | 958 |
| | | Nei-65470 | 65450-65470 | GAAGGTTACCTAGGTCCGGTC | 959 |
| | | Nei-65476 | 65455-65476 | TTACCTAGGTCCGTCGGAAAT | 960 |
| | | Nei-65491 | 65466-65491 | CGGTCGGAAATCGGACTGATAGTGCA | 961 |
| | | Nei-65498 | 65475-65498 | ATCGGACTGATAGTGCAATGGCAA | 962 |
| | | Nei-65633 | 65605-65633 | TCCGGGGATAACAGGCTGATTCCGCCCAA | 963 |
| | Peptococus | Ptc-31 | 4-31 | TAAGTAACTAAGGGCATGCGGTGAATGC | 964 |
| | | Ptc-105 | 80-105 | GTTGAGTCGCAAGCAGACCTTGACAC | 965 |
| | | Ptc-117 | 88-117 | GCAAGCAGACCTTGACACGTAGATATCCGA | 966 |
| | | Ptc-124 | 103-124 | CACGTAGATATCCGAATAGGAC | 967 |
| | | Ptc-136 | 109-136 | GATATCCGAATAGGACAACCCTGCCGGA | 968 |
| | | Ptc-144 | 122-144 | GACAACCCTGCCGGAGTTATGTC | 969 |
| | | Ptc-156 | 134-156 | GGAGTTATGTCCGGCAACCTTAA | 970 |
| | | Ptc-166 | 139-166 | TATGTCCGGCAACCTTAAAGCCAATCCA | 971 |
| | | Ptc-183 | 163-183 | TCCATAACTTTAAGGAGGGCA | 972 |
| | | Ptc-188 | 167-188 | TAACTTTAAGGAGGGCAACCCG | 973 |
| | | Ptc-248 | 222-248 | GAAAGAAAACTCGATTCCCCCAGTAGC | 974 |
| | | Ptc-284 | 263-284 | GAAGAGCCCAAACCGAGCAATC | 975 |
| | | Ptc-292 | 271-292 | CAAACCGAGCAATCGGGGTAAG | 976 |
| | | Ptc-305 | 277-305 | GAGCAATCGGGGTAAGGACACTCAAAAAG | 977 |
| | | Prc-362 | 339-362 | CAGCCATAGAAGGTTAAAGCCCTG | 978 |
| | | Ptc-392 | 372-392 | AGACAAAATATCCGGAGTGGA | 979 |
| | | Ptc-398 | 375-398 | CAAAATATCCGGAGTGGATCCGGA | 980 |
| | | Ptc-419 | 400-419 | TACCACGAGGCACGAGGAAT | 981 |
| | | Ptc-430 | 410-430 | CACGAGGAATCTCGTGGGAAG | 982 |
| | | Ptc-468 | 440-468 | CCACCCCCCAAGGCTAAATACTCCCCGGC | 983 |
| | | Ptc-533 | 509-533 | CACCTCGGAAGAGGAGTGAAATAGA | 984 |
| | | Ptc-557 | 529-557 | ATAGAACCTGAAACCGCATGCTTACAATC | 985 |
| | | Ptc-563 | 540-563 | AACCGCATGCTTACAATCAGTCAC | 986 |
| | | Ptc-583 | 560-583 | TCACAGCTCCACATGCGAGTAGTG | 987 |
| | | Ptc-595 | 569-595 | CACATGCGAGTAGTGGCGTACTTTTTG | 988 |
| | | Ptc-611 | 588-611 | ACTTTTTGTAGAACGGACCGGCGA | 989 |
| | | Ptc-681 | 654-681 | AAGCAAGTCTTAATAGGGCGCAAGTTTC | 990 |
| | | Ptc-686 | 665-686 | AATAGGGCGCAAGTTTCTTGG | 991 |
| | | Ptc-698 | 672-698 | CGCAAGTTTCTTGGCGCAGACCCGAAA | 992 |
| | | Ptc-717 | 695-717 | GAAACCGGGTGATCTACCCATGA | 993 |
| | | Ptc-729 | 701-729 | GGGTGATCTACCCATGAGCAGGTTGAAGC | 994 |
| | | Ptc-740 | 712-740 | CCATGAGCAGGTTGAAGCGTTGGTAAAAC | 995 |
| | | Ptc-744 | 725-744 | GAAGCGTTGGTAAAACAACG | 996 |
| | | Ptc-777 | 751-777 | ACCGAACCAGGTGTCGTTGAAAAGACA | 997 |
| | | Ptc-798 | 773-798 | AGACATTGGATGACTTGTGGGTAGGG | 998 |
| | | Ptc-929 | 903-929 | GGCTTCACCGCCTACCAAATCTTATCA | 999 |
| | | Ptc-950 | 927-950 | TCAAACTCAGAATGCCGTAGGGGA | 1000 |
| | | Ptc-958 | 933-958 | TCAGAATGCCGTAGGGGAGTTACTTG | 1001 |
| | | Ptc-970 | 942-970 | CGTAGGGGAGTTACTTGGGAGTCAGACTA | 1002 |
| | | Ptc-975 | 953-975 | TACTTGGGAGTCAGACTATGGGG | 1003 |
| | | Ptc-987 | 959-987 | GGAGTCAGACTATGGGGGATAAGCTTCAT | 1004 |
| | | Ptc-992 | 970-992 | ATGGGGGATAAGCTTCATAGTCA | 1005 |
| | | Ptc-1014 | 988-1014 | AGTCAAAAGGGAAAGAACCCAGACCGT | 1006 |
| | | Ptc-1022 | 998-1022 | GAAAGAACCCAGACCGTCGTCTAAG | 1007 |
| | | Ptc-1063 | 1044-1063 | GGAAAAGGATGTAGAATCCC | 1008 |
| | | Ptc-1070 | 1048-1070 | AAGGATGTAGAATCGCTCAGACA | 1009 |
| | | Ptc-1107 | 1088-1107 | GAAGCAGCCATCATTAAGAG | 1010 |
| | | Ptc-1114 | 1090-1114 | AGCAGCCATCATTAAGAGAGTGCGT | 1011 |
| | | Ptc-1156 | 1134-1156 | GGTTCCGCGCCGAAAATGTAACG | 1012 |
| | | Ptc-1172 | 1147-1172 | AAATGTAACGGGGCTCAAACACCACA | 1013 |
| | | Ptc-1183 | 1158-1183 | GGCTCAAACACCACACCGAAGACACG | 1014 |
| | | Ptc-1237 | 1218-1237 | AGAACGAAGCGAGAGCGCAA | 1015 |
| | | Ptc-1280 | 1261-1280 | GAGAATGCCGGTATAAGTAC | 1016 |
| | | Ptc-1286 | 1263-1286 | GAATGCCGGTATAAGTACACGATA | 1017 |
| | | Ptc-1310 | 1288-1310 | AAGAGGTGAGAATCCTCTTCGCC | 1018 |
| | | Ptc-1411 | 1392-1411 | GGGAAACAGGTAGAAATTCC | 1019 |
| | | Ptc-1417 | 1398-1417 | CAGGTAGAAATTCCTGTACC | 1020 |
| | | Ptc-1447 | 1425-1447 | TTTAATGACCACGCAGTGACGCA | 1021 |
| | | Ptc-1458 | 1438-1458 | CAGTGACGCAGAAGGGTACAG | 1022 |
| | | Ptc-1487 | 1466-1487 | CCAGTTGGTGAGGCGTTCAAGC | 1023 |
| | | Ptc-1511 | 1491-1511 | TAGGGCGATGCGTAGGCAAAT | 1024 |
| | | Ptc-1522 | 1502-1522 | GTAGGCAAATCCGCGCATCAC | 1025 |
| | | Ptc-1537 | 1518-1537 | ATCACGAGCCTGAGACGTGA | 1026 |
| | | Ptc-1555 | 1528-1555 | TGAGACGTGACGACGAGTGAAACATAGT | 1027 |
| | | Ptc-1575 | 1553-1575 | AGTAACGAAGTCCTGAATCCCCC | 1028 |
| | | Ptc-1588 | 1568-1588 | AATCCCCCGCTGCCAAGAAAA | 1029 |
| | | Ptc-1599 | 1575-1599 | CGCTGCCAAGAAAAGCTGCTAAGGA | 1030 |
| | | Ptc-1624 | 1600-1624 | AAATAAGATAGCCCGTACCGCAAAC | 1011 |
| | | Ptc-1651 | 1632-1651 | GGTAGACAGGTAGAGAATAC | 1032 |
| | | Ptc-1677 | 1656-1677 | GCGCGCGAGATAACTCTTGTTA | 1033 |
| | | Ptc-1705 | 1678-1705 | AGGAACTCGGCAAAATGAACCCGTAACT | 1034 |
| | | Ptc-1737 | 1718-1737 | TTGCTCCCCCGTGTGAGTAT | 1035 |
| | | Ptc-1744 | 1721-1744 | CTCCCCCGTGTGAGTATGAACAAT | 1036 |
| | | Ptc-1963 | 1741-1763 | CAATACAGAGCATAAGGGAGCCG | 1037 |
| | | Ptc-1772 | 1751-1772 | CATAAGGGAGCCGCAGAGAAGA | 1038 |
| | | Ptc-1791 | 1769-1791 | AAGAGGTCCAGGCGACTGTTTAG | 1039 |
| | | Ptc-1796 | 1774-1796 | GTCCAGGCGACTGTTTAGCAAAA | 1040 |
| | | Ptc-1811 | 1791-1811 | GCAAAAACACAGGTCATTGCC | 1041 |
| | | Ptc-1821 | 1797-1821 | ACACAGGTCATTGCCAAATCGTAAG | 1042 |
| | | Ptc-1828 | 1808-1828 | TGCCAAATCGTAAGATCACGT | 1043 |
| | | Ptc-1847 | 1821-1847 | GATCACGTATAATGGCTGACGCCTGCC | 1044 |
| | | Ptc-2004 | 1975-2004 | CGTAACGATCTGGACGCTGTCTGAACAAGG | 1045 |
| | | Ptc-2028 | 2009-2028 | CGGTGAAATTGAATTACCGG | 1046 |
| | | Ptc-2104 | 2076-2104 | TTACTGGACCCTGATATTGGGTTTCGGTT | 1047 |
| | | Ptc-2132 | 2107-2132 | TTATGTACAGCATAGGTGGGAGACAG | 1048 |
| | | Ptc-2137 | 2117-2137 | CATAGGTGGGAGACAGCGAAG | 1049 |
| | | Ptc-2154 | 2134-2154 | GAAGCGAGAGCGCCAGTTTTC | 1050 |
| | | Ptc-2161 | 2140-2161 | AGAGCGCCAGTTTTCGCAGAGT | 1051 |
| | | Ptc-2170 | 2144-2170 | CGCCAGTTTTCGCAGAGTCACCCTTGG | 1052 |
| | | Ptc-2178 | 2155-2178 | GCAGAGTCACCCTTGGGATACCAC | 1053 |
| | | Ptc-2189 | 2163-2189 | ACCCTTGGGATACCACCCTTAAGTGAT | 1054 |
| | | Ptc-2237 | 2217-2237 | GGTTATTGGACATTGTCAGGC | 1055 |
| | | Ptc-2242 | 2220-2242 | TATTGGACATTGTCAGGCAGGCA | 1056 |
| | | Ptc-2251 | 2225-2251 | GACATTGTCAGGCAGGCAGTTTGACTG | 1057 |
| | | Ptc-2308 | 2282-2308 | CGCTCAAAGGTTCCCTCAGAACGGATG | 1058 |
| | | Ptc-2319 | 2291-2319 | GTTCCCTCAGAACGGATGGAAATCGTTCA | 1059 |
| | | Ptc-2322 | 2303-2322 | CGGATGGAAATCGTTCATAG | 1060 |
| | | Ptc-2333 | 2315-2333 | GTTCATAGAGTGTAAAGGC | 1061 |
| | | Ptc-2390 | 2371-2390 | GGAAGGAAACTTGGACTTAG | 1062 |
| | | Ptc-2400 | 2378-2400 | AACTTGGACTTAGTGATCCGGCG | 1063 |
| | | Ptc-2404 | 2384-2404 | GACTTAGTGATCCGGCGGTTG | 1064 |
| | | Ptc-2625 | 2596-2625 | TGAGACAGTTCGGTCCCTATCCATCGTAG | 1065 |
| | | Ptc-2637 | 2610-2637 | TCCCTATCCATCGTAGGCGTAGGATATT | 1066 |
| | | Ptc-2641 | 2620-2641 | TCGTAGGCGTAGGATATTTGAG | 1067 |
| | | Ptc-2647 | 2624-2647 | AGGCGTAGGATATTTGAGAGGCAC | 1068 |
| | | Ptc-2667 | 2644-2667 | GCACTGACCCTAGTACGAGAGGAC | 1069 |
| | | Ptc-2692 | 2671-2692 | GTTGGACACACCGCTGGTTAAC | 1070 |
| | | Ptc-2699 | 2678-2699 | ACACCGCTGGTTAACCGGTTGT | 1071 |
| | | Ptc-2708 | 2683-2708 | GCTGGTTAACCGGTTGTCGTGCCAAC | 1072 |
| | | Ptc-2732 | 2710-2732 | GCATAGCCGGGTAGCTAAGTGTG | 1073 |
| | | Ptc-2742 | 2718-2742 | GGGTAGCTAAGTGTGGCCATGATAA | 1074 |
| | | Ptc-2753 | 2726-2753 | AAGTGTGGCCATGATAAACGCTGAAAGC | 1075 |
| | | Ptc-2777 | 2748-2777 | GAAAGCATCTAAGCGTGAAGCAGCCCTCAA | 1076 |
| | | Ptc-2797 | 2777-2797 | AGATGAGATATCCCACTAGGA | 1077 |
| | | Ptc-2800 | 2781-2800 | GAGATATCCCACTAGGATAC | 1078 |
| | | Ptc-2815 | 2794-2815 | AGGATACTAGATAAGACCCCAG | 1079 |
| | | Ptc-2826 | 2798-2826 | TACTAGATAAGACCCCAGAGAGACGAACT | 1080 |
| | | Ptc-2853 | 2828-2853 | GTAGATAGGTCGGGCGTGTAAGAAGA | 1081 |
| | | Ptc-2860 | 2837-2860 | TCGGGCGTGTAAGAAGAGCAATCT | 1082 |
| | Plesiomonas | Ple-63 | 44-63 | AGGCGATGAAGGACGTCGAT | 1083 |
| | | Ple-68 | 48-68 | GATGAAGGACGTCGATGGGAC | 1084 |
| | | Ple-75 | 55-75 | GACGTCGATGGGACGGAAAAG | 1085 |
| | | Ple-82 | 59-82 | TCGATGGGACGGAAAAGGGTTGGT | 1086 |
| | | Ple-92 | 65-92 | GGACGGAAAAGGGTTGGTGAGCTGGGAT | 1087 |
| | | Ple-103 | 84-103 | AGCTGGGATGGAGCGCTATA | 1088 |
| | | Ple-131 | 106-131 | CAACCATGTCCGAATGGGGAAACCCA | 1089 |
| | | Ple-142 | 116-142 | CGAATGGGGAAACCCACCTAAGATAAC | 1090 |
| | | Ple-147 | 128-147 | CCCACCTAAGATAACTTAGG | 1091 |
| | | Ple-186 | 164-186 | CATAGCTTAATGAGGCGAACCGG | 1092 |
| | | Ple-220 | 194-220 | CAAACATCTAAGTACCCCAGGAAAAGA | 1093 |
| | | Ple-224 | 205-224 | GTACCCGAGGAAAAGAAATC | 1094 |
| | | Ple-304 | 278-304 | ATCAGTGGATGTGTTAGTGGAACGGAT | 1095 |
| | | Ple-309 | 287-309 | TGTGTTAGTGGAACGGATTGGAA | 1096 |
| | | Ple-317 | 292-317 | TAGTGGAACGGATTGGAAAGTCCGGC | 1097 |
| | | Ple-324 | 301-324 | GGATTGGAAAGTCCGGCGATACAG | 1098 |
| | | Ple-348 | 326-348 | GTGATAGCCCCGTACACGAAGAC | 1099 |
| | | Ple-364 | 334-364 | CCCGTACACGAAGACGGATTGCTGTGAGCTC | 1100 |
| | | Ple-390 | 371-390 | TAGGACGGGACACGTGGTAT | 1101 |
| | | Ple-418 | 389-418 | ATCCTGTTTGAAGATAGGGGGGACCATCCT | 1102 |
| | | Ple-467 | 444-467 | CGATAGCGAACCAGTACCGTGAAG | 1103 |
| | | Ple-475 | 451-475 | GAACCAGTACCGTGAAGGGAAAGGC | 1104 |
| | | Ple-513 | 486-513 | CCTGTGAGGGGAGTGTGAAATAGAACCT | 1105 |
| | | Ple-543 | 514-543 | AAAACCGTGTACGTACAAGCAGTGGGAGCA | 1106 |
| | | Ple-666 | 643-666 | TAACTGGGCGAATTAGTTGCACCC | 1107 |
| | | Ple-674 | 649-674 | GGCGAATTAGTTCCACCCGAAACCCG | 1108 |
| | | Ple-940 | 917-940 | GTAGAGTGCTATGCGGGAGACACA | 1109 |
| | | Ple-1173 | 1146-1173 | CGAAGCTGCGGCAATGACATTTAGGTGT | 1110 |
| | | Ple-1190 | 1166-1190 | TTAGGTGTTATTGGGCTAGGGGAGC | 1111 |
| | | Ple-1193 | 1173-1193 | TTATTGGGCTAGGGGAGCGTT | 1112 |
| | | Ple-1201 | 1180-1201 | GCTAGGGGAGCGTTCTGTAAGC | 1113 |
| | | Ple-1419 | 1390-1419 | ATTCCCGTACTTGTGGTAACTGCGAAGGGG | 1114 |
| | | Ple-1434 | 1414-1434 | AAGGGGGGACGGAGAAAGTTA | 1115 |
| | | Ple-1448 | 1419-1448 | GGGACGGAGAAAGTTAGGCTATCCGGGCGA | 1116 |
| | | Ple-1512 | 1493-1512 | CCGGTCTTTCATTAACACTG | 1117 |
| | | Ple-1517 | 1498-1517 | CTTTCATTAACACTGAGGCG | 1118 |
| | | Ple-1540 | 1513-1540 | AGGCGTGATGACGAAGCACTACGGTGCT | 1119 |
| | | Ple-1560 | 1538-1560 | GCTGAAGTAGCTGATACTACGCT | 1120 |
| | | Ple-1569 | 1543-1569 | AGTAGCTGATACTACGCTTCCAGGAAA | 1121 |
| | | Ple-1599 | 1579-1599 | GCATCAGGTTACGAGAAATCG | 1122 |
| | | Ple-1722 | 1703-1722 | ACGCTGACGGTAGGTGAAGT | 1123 |
| | | Ple-1728 | 1707-1728 | TGACGGTAGGTGAAGTCCCTTG | 1124 |
| | | Ple-1746 | 1725-1746 | CTTGCGGATGGAGCTGAAGTCA | 1125 |
| | | Ple-2208 | 2185-2208 | TGATGTTCTAACGTTGTCCCGTAA | 1126 |
| | | Ple-2213 | 2191-2213 | TCTAACGTTGTCCCCTAATCCGG | 1127 |
| | | Ple-2218 | 2197-2318 | GTTGTCCCGTAATCCGGGATAC | 1128 |
| | | Ple-2227 | 2204-2227 | CGTAATCCGGGATACGGACAGTGT | 1129 |
| | | Ple-2234 | 2213-2234 | GCATACGGACAGTGTCTGGTGG | 1130 |
| | Porphyromonas | Porp-121942 | 121918-121942 | CTGCGAAAAGCTGCGGGAATCGGCA | 1131 |
| | | Porp-121953 | 121931-121953 | CGGGAATCGGCACATAGGAATTG | 1132 |
| | | Porp-121962 | 121937-121962 | TCGGGACATACGAATTGATCGGCAGA | 1133 |
| | | Porp-121974 | 121947-121974 | CGAATTGATCCGCAGATATCGGAATGGG | 1134 |
| | | Porp-121981 | 121958-121981 | GCAGATATCCGAATGGGGCAACCC | 1135 |
| | | Porp-122011 | 121987-122011 | GCCAAGGCCTGACACATGAATTGAT | 1136 |
| | | Porp-122029 | 122007-122029 | TTGATTTCATGAGCGAACGCGGC | 1137 |
| | | Porp-122046 | 122021-122046 | GAACGCGGGGAACTGAAACATCTCAT | 1138 |
| | | Porp-122052 | 122033-122052 | CTGAAACATCTCATTACCCG | 1139 |
| | | Porp-122063 | 122037-122063 | AACATCTCATTACCCGTAGGAGAAGAA | 1140 |
| | | Porp-122093 | 122069-122093 | AAGTGATTCCCTCAGTAGTGGCGAG | 1141 |
| | | Porp-122115 | 122094-122115 | CGAACGGGGATTAGCCCAAACC | 1142 |
| | | Porp-122189 | 122170-122189 | GATATAGGAGAACCTACTGG | 1143 |
| | | Porp-122195 | 122174-122195 | TAGGAGAACCTACTGGAAAGTA | 1144 |
| | | Porp-122257 | 122229-122257 | TATCGAACAATAGACGACGGAGCACCTGA | 1145 |
| | | Porp-122319 | 122297-122319 | GCCCATCCCGTAAGGCTAAATAC | 1146 |
| | | Porp-122379 | 122358-122379 | AGGTGAAAAGAACCTCGAACAG | 1147 |
| | | Porp-122416 | 122396-122416 | CTGAACCCGTCTCCCTACAAG | 1148 |
| | | Porp-122424 | 122405-122424 | TCTGCCTACAAGCGCTAGGA | 1149 |
| | | Porp-122434 | 122414-122434 | AAGCGGTAGGAGCGCCATTAA | 1150 |
| | | Porp-122491 | 122465-122491 | CCTACGAGTTACTGTTTGTGGCAAGGT | 1151 |
| | | Porp-122523 | 122508-122523 | ATAATCAAGACAAGGAGCGGAAGG | 1152 |
| | | Porp-122528 | 122506-122528 | AAGACAAGGAGCCGAAGCGAAAG | 1153 |
| | | Porp-122558 | 122530-122558 | GAGTCTTAAAAGGGCGCCCATTTAGTCAC | 1154 |
| | | Porp-122571 | 122544-122571 | CGCCCATTTAGTCACGAGCAGTAGACGC | 1155 |
| | | Porp-122576 | 122555-122576 | TCACGAGCAGTAGACGCGAAAC | 1156 |
| | | Porp-122649 | 122625-122649 | AGGACCGAATCGGTAAGCGTTGAAA | 1157 |
| | | Porp-122654 | 122633-122654 | ATCGGTAAGCGTTGAAAAGCTT | 1158 |
| | | Porp-122671 | 122650-122671 | AGCTTTCGAATGAACTGAGGGT | 1159 |
| | | Porp-122682 | 122654-122682 | TTCGAATGAACTGAGGGTAGGGGTGAAAG | 1160 |
| | | Porp-122693 | 122667-122693 | AGGGTAGGGGTGAAAGGGTAATCAAAC | 1161 |
| | | Porp-122753 | 122734-122753 | GCCTGTTGGATGTTATGATG | 1162 |
| | | Porp-122788 | 122763-122788 | GACTGATTGGATGCGAGGGTTTCACC | 1163 |
| | | Porp-122814 | 122794-122814 | TCAAGTCCAGATAAAGTCCGA | 1164 |
| | | Porp-122819 | 122798-122819 | GTCCAGATAAACTCGGAATGCA | 1165 |
| | | Porp-122840 | 122817-122840 | GCATGATAATTGACCGATGGAGTG | 1166 |
| | | Porp-122844 | 122823-122844 | TAATTGACCGATGGAGTGAGGG | 1167 |
| | | Porp-122874 | 122850-122874 | TGCTAAGGTCGATGTCCGAGAGGA | 1168 |
| | | Porp-122884 | 122858-122884 | GTCCATGTCCGAGAGGAGAAGAATCCG | 1169 |
| | | Porp-122890 | 122867-122890 | CGAGAGGAGAAGAATCCGGACCAC | 1170 |
| | | Porp-122911 | 122889-122911 | ACCGGCTAAGGTCCCGAAATAAT | 1171 |
| | | Porp-123043 | 123016-123043 | TTGGCATGGATAATACACGGGCATAAGC | 1172 |
| | | Porp-123053 | 123031-123053 | CACGGGCATAAGCAATTTACCGA | 1173 |
| | | Porp-123094 | 123068-123094 | GTAATATATCGGTAGGGGAGCATTCCA | 1174 |
| | | Porp-123144 | 123124-123144 | TCTGGAGTTTCTGGAAAAGCA | 1175 |
| | | Porp-123180 | 123155-123180 | TAAGTAAGGATAAAGGGGGCGAGAAC | 1176 |
| | | Porp-123210 | 123135-123210 | CTCGGCGAAAGACCAAGGTTTGCTGA | 1177 |
| | | Porp-123253 | 123231-123253 | GTTAGTCGGGGCCTAAGGATAAG | 1178 |
| | | Porp-123299 | 123278-123299 | ACCGGTTAATATTCCGGTACTG | 1179 |
| | | Porp-123327 | 123308-123327 | AGCGATGTGGTGACGGAGAA | 1180 |
| | | Porp-123337 | 123310-123337 | CGATGTGGTGACGGAGAAGTGACAGTCC | 1181 |
| | | Porp-123405 | 123386-123405 | TAGGCAAATCCGCCCTGAGA | 1182 |
| | | Porp-123411 | 123391-123411 | AAATCGGCCGTGAGAGTCGAA | 1183 |
| | | Porp-123424 | 123399-123434 | CCTGAGAGTCGAACCTGACAGTACCC | 1184 |
| | | Porp-123430 | 123410-123430 | AACCTGACAGTAGCGGGAGTA | 1185 |
| | | Porp-123474 | 123450-123474 | ACGTAAACCGGCTCCCAAGAAAACC | 1186 |
| | | Porp-123484 | 128460-123484 | GCTGCCAAGAAAACCCGCTAAGCAT | 1187 |
| | | Porp-123493 | 128472-123493 | ACCCGCTAAGCATATTTCTGTG | 1188 |
| | | Porp-123500 | 123481-123500 | GCATATTTCTGTGTTACCCG | 1189 |
| | | Porp-123526 | 123504-123526 | CGTAAACCGACACAGGTGGTTGG | 1190 |
| | | Porp-123538 | 123512-123538 | GACACAGGTGGTTGGGTTGAGTATACT | 1191 |
| | | Porp-123591 | 128564-123591 | AGGAACTAGGCAAAATGGTCCTGTAACT | 1192 |
| | | Porp-123705 | 123686-123705 | GAGGTATATAGTCTGACACC | 1193 |
| | | Porp-123711 | 123088-123711 | GGTATATAGTCTGACACCTGCCCG | 1194 |
| | | Porp-123881 | 123861-123881 | CACTGTCTCAACCGCGATCTC | 1195 |
| | | Porp-123894 | 123868-123894 | TCAACCGCGATCTCGGTGAAATTGTAG | 1196 |
| | | Porp-123933 | 128912-123933 | ATTACCCGCAACGGGACGAAAA | 1197 |
| | | Porp-123985 | 123966-123985 | GTATTTGGGCATCAGATGTG | 1198 |
| | | Porp-124026 | 124005-124026 | AGAAGGGGGTACGCCAGTATTC | 1199 |
| | | Porp-124034 | 124012-124034 | GGTACGCCAGTATTCGTGGAGTC | 1200 |
| | | Porp-124045 | 124017-124045 | GCCAGTATTCGTGGAGTCGATGTTGAAAT | 1201 |
| | | Porp-124054 | 124029-124054 | GGAGTCGATGTTGAAATACGGCCCTT | 1202 |
| | | Porp-124060 | 124038-124060 | GTTGAAATACGGCCCTTTTGATG | 1203 |
| | | Porp-124067 | 124045-124067 | TACGGCCCTTTTGATGTTTGGAT | 1204 |
| | | Porp-124082 | 124059-124082 | TGTTTGGATACTAACTCGCGGCGT | 1205 |
| | | Porp-124101 | 124081-124101 | GTGCGAGGACAGTGTATGGTG | 1206 |
| | | Porp-124140 | 124118-124140 | TGGTCGCCTCCAAAAGCGTAACG | 1207 |
| | | Porp-124170 | 124149-124170 | TAAAGGTACCCTCAGGCCGATT | 1208 |
| | | Porp-124175 | 124155-124175 | TACCCTCAGGCCGATTGGTAA | 1209 |
| | | Porp-124188 | 124167-124188 | GATTGGTAACCGGTCGCAGAGT | 1210 |
| | | Porp-124194 | 124172-124194 | GTAACCGGTCGCAGAGTGTAATG | 1211 |
| | | Porp-124218 | 124197-124218 | ACAAGGGTGCTTGACTGGGAGA | 1212 |
| | | Porp-124269 | 124241-124269 | AACTAGAGCATAGTGATCCGGTGGTTCCG | 1213 |
| | | Porp-124336 | 124307-124336 | CGGGGATAACAGGCTGATCACTCCCAAGAG | 1214 |
| | | Porp-124371 | 124349-124371 | GAGTGGTTTGGCACCTCGATGTG | 1215 |
| | | Porp-124505 | 124477-124505 | TATCTGTTGTGGGCGCAGGAAATTTGCGA | 1216 |
| | | Porp-124543 | 124524-124543 | AGAGGACCGTGTTGGACAGA | 1217 |
| | | Porp-124555 | 124533-124555 | TGTTGGACAGACCCCTGGTTTAC | 1218 |
| | | Porp-124560 | 124541-124560 | AGACCCCTGGTTTACCGGTT | 1219 |
| | | Porp-124570 | 124543-124570 | ACCCCTGGTTTACCGGTTGTACCGCCAG | 1220 |
| | Propion- | Prop-519 | 498-519 | AAGGCCAATCAAACACCGTGAT | 1221 |
| | bacterium | Prop-1834 | 1808-1834 | ACTGTCTCCACCATGAACTCGGTGAAA | 1222 |
| | | Prop-2012 | 1989-2012 | ATACCACTCTGGTCGTTCTGGTTA | 1223 |
| | | Prop-2014 | 1995-2014 | CTCTGGTCGTTCTGGTTATC | 1224 |
| | Providencia | Prov-127 | 106-127 | TCGGTCATTCAAACGAGTGGCA | 1225 |
| | | Prov-135 | 113-135 | TTCAAACGAGTGGCATGAGCGAG | 1226 |
| | | Prov-206 | 179-206 | ACAGGAGTACGTGAGCATTGCGACCACT | 1227 |
| | | Prov-231 | 208-231 | CCCAACGCAGAAGTTCACCACGCA | 1228 |
| | | Prov-235 | 214-235 | GCAGAAGTTCACCAGGCACAGC | 1229 |
| | | Prov-238 | 218-238 | AAGTTCACCACGCACAGCCAT | 1230 |
| | | Prov-240 | 221-240 | TTCACCACGCACAGCCATGA | 1231 |
| | | Prov-246 | 227-246 | ACGCACAGCCATGACAGTCA | 1232 |
| | | Prov-255 | 230-255 | CACAGCCATGACAGTCAGGTGATCGT | 1233 |
| | | Prov-258 | 238-258 | TGACAGTCAGGTGATCGTTGA | 1234 |
| | | Prov-831 | 811-831 | GGGAGCCTTGATTTATCAGGG | 1235 |
| | | Prov-839 | 820-839 | GATTTATCAGGGTGACTGCG | 1236 |
| | | Prov-849 | 823-849 | TTATCAGGGTGACTGCGTACCTTTTGT | 1237 |
| | Pseudomonas | Pseu-20 | 1-20 | GGTCAAGTGAAGAAGCGCAT | 1238 |
| | | Pseu-2039 | 2017-2039 | GTGAAGATGCAGTGTATCCGCGG | 1239 |
| | | Pseu-2042 | 2023-2042 | ATGCAGTGTATCCGCGGCTA | 1240 |
| | | Pseu-2050 | 2031-2050 | TATCCGCGGCTAGACCGAAA | 1241 |
| | | Pseu-2118 | 2095-2118 | TGCTTGTGTAGGATAGGTGGGAGG | 1242 |
| | | Pseu-2290 | 2271-2290 | AGTACGAAGGTGCGCTCAGA | 1243 |
| | | Pseu-2338 | 2316-2338 | TAAAGGCAAAAGCGCGCTTGACT | 1244 |
| | | Pseu-2628 | 2601-2628 | CCTATCTGCCGTGGACGTTTGAGATTTG | 1245 |
| | | Pseu-2636 | 2616-2636 | CGTTTGAGATTTGAGAGGGGC | 1246 |
| | | Pseu-2641 | 2619-2641 | TTGAGATTTGAGAGGGGCTGCTC | 1247 |
| | | Pseu-2644 | 2624-2644 | ATTTGAGAGGGGCTGCTCCTA | 1248 |
| | | Pseu-2773 | 2751-2773 | GGGAAACTTGCCTCAAGATGAGA | 1249 |
| | | Pseu-2779 | 2260-2779 | GCCTCAAGATGAGATCTCAC | 1250 |
| | | Pseu-2819 | 2798-2819 | AAGGGCCGTCGAAGACTACGAC | 1251 |
| | | Pseu-2865 | 2845-2865 | TGTGAGGCGTTGAGCTAACCA | 1252 |
| | | Pseu-2893 | 2872-2893 | ATTGCCCGTGAGGCTTGACCAT | 1253 |
| | Salmonella | Sal-303 | 288-303 | GCATGTGTGTTAGTGGAAGCG | 1254 |
| | | Sal-1726 | 1706-1726 | CACGCTGACACGTAGGTGAAG | 1255 |
| | | Sal-1719 | 1700-1719 | AGAAGGCACGCTGACACGTA | 1256 |
| | Shigella | Shi-216512 | 216490-216512 | ATGCACATACTGTGAGCTCGATG | 1257 |
| | | Shi-216514 | 216495-216514 | CATACTGTGAGCTCGATGAG | 1258 |
| | | Shi-216519 | 216498-216519 | ACTGTGAGCTCGATGAGTAGGG | 1259 |
| | | Shi-216689 | 216670-216689 | AAGCAGTGGGAGCACGCTTA | 1260 |
| | | Shi-216706 | 216686-216706 | CTTAGGCGTGTGACTGCGTAC | 1261 |
| | | Shi-216712 | 216691-216712 | GCGTGTGACTGCGTACCTTTTG | 1262 |
| | | Shi-217022 | 217000-217022 | GGTAGAGCACTGTTTCGGCAAGG | 2263 |
| | | Shi-217029 | 217010-217029 | TGTTTCGGCAAGGGGGTCAT | 1264 |
| | | Shi-217388 | 217363-217388 | CTGTGAGGTATGCTGGAGGTATCAGA | 1265 |
| | | Shi-217658 | 217637-217658 | AATCCGGAAAATCAAGGCCGAG | 1266 |
| | | Shi-217665 | 217643-217665 | GAAAATCAAGGCCGAGGCGTGAT | 1267 |
| | Staphylococcus | Sta-23 | 1-23 | GATTAAGTTATTAAGGGCGCACG | 1268 |
| | | Sta-77 | 57-77 | CGTTACTAACGACGATATGCT | 1269 |
| | | Sta-277 | 257-277 | GAAACGGGAAGAGCCCAAACC | 1270 |
| | | Sta-423 | 404-423 | TGAGTGGATCCTGAGTACGA | 1271 |
| | | Sta-496 | 477-496 | TACTCTCTAGTGACCGATAG | 1272 |
| | | Sta-596 | 575-596 | GTAGTCAGAGCCCGTTAATGGG | 1273 |
| | | Sta-719 | 700-719 | AGTATTTGGTCGTAGACCCG | 1274 |
| | | Sta-1168 | 1149-1168 | TAGTCGAGTGACACTGCGCC | 1275 |
| | | Sta-1283 | 1263-1283 | AAGGAGATGTGGAGCGCTTAG | 1276 |
| | | Sta-1349 | 1330-1349 | TCCACCGATTGACTAAGGTT | 1277 |
| | | Sta-1639 | 1673-1695 | ATGAGAATTCTAAGGTGAGCGAG | 1278 |
| | | Sta-1763 | 1741-1763 | GAGAAGGGGTGCTCTTTAGGGTT | 1279 |
| | | Sta-1792 | 1772-1792 | GAAGAGCCGCAGTGAATAGGC | 1280 |
| | | Sta-1909 | 1888-1909 | TGGTTAGCTTCTGCGAAGCTAC | 1281 |
| | | Sta-2065 | 2044-2065 | CATAGTACCTGTGAAGATGCAG | 1282 |
| | | Sta-2135 | 2116-2135 | TATTGAAATTCGGCACAGCT | 1283 |
| | | Sta-2214 | 2194-2214 | TGGGATACTACCCTAGCTGTG | 1284 |
| | | Sta-2415 | 2396-2415 | AGGGTCGAAAGACGGACTTA | 1285 |
| | | Sta-2836 | 2814-2836 | CCCAACTTCGGTTATAAGATCCC | 1286 |
| | Streptococcus | Str-409 | 389-409 | CCTAGCAGTATCCTGAGTACG | 1287 |
| | | Str-648 | 627-648 | GATGCGAGGTTAAGTTGAAGAG | 1288 |
| | | Str-791 | 770-791 | CAGGGCACGTTGAAAAGTGCTT | 1289 |
| | | Str-1389 | 1367-1389 | GGTTAGTCGGGATCTAAGGAGAG | 1290 |
| | | Str-1593 | 1573-1593 | GCGAAGTTAGTGACGTCACAC | 1291 |
| | | Str-1667 | 1648-1667 | ACAGGTAGTCGAGGCGAGTA | 1292 |
| | | Str-2199 | 2180-2199 | CCCTTGTGTTATGGGTACTC | 1293 |
| | | Str-2226 | 2207-2226 | GATAGGTKATCCCTATCGGA | 1294 |
| | | Str-2253 | 2228-2253 | ACAGTGTCTGACGGGCAGTTTGACTG | 1295 |
| | | Str-2345 | 2326-2345 | GTGTAAAGGTATAAGGGAGC | 1296 |
| | | Str-2652 | 2633-2652 | GGAAATTTGAGAGGATCTGC | 1297 |
| | | Str-2822 | 2803-2822 | ATCAGTAAGAGCCCTGAGAG | 1298 |
| | Treponema | Tre-231971 | 231950-231971 | GCGAATAGTGGTTTACGGTGGA | 1299 |
| | | Tre-232049 | 232026-232049 | GAGGAGCACATGTCCTGTGATCCG | 1300 |
| | | Tre-232054 | 292035-232054 | ATGTCCTGTGATCCGGGGAT | 1301 |
| | | Tre-232062 | 232043-232062 | TGATCCGGGGATGACCGAAT | 1302 |
| | | Tre-232072 | 232050-232072 | GGGATGACCGAATGGGCTAACCC | 1303 |
| | | Tre-292091 | 232064-232091 | CGGTAACCCCACAGGGTAAAGCCTTGTC | 1304 |
| | | Tre-232098 | 232071-232098 | CCGACAGGGTAAAGCCTTGTCATTGCCT | 1305 |
| | | Tre-232108 | 232081-232108 | AAAGCCTTGTCATTGCCTTCCTGAATGA | 1306 |
| | | Tre-232120 | 232094-232120 | TGCCTTCCTGAATGAATAGGGAGGGTA | 1307 |
| | | Tre-232130 | 232103-232130 | GAATGAATAGGGAGGGTAAGGCGAAACT | 1308 |
| | | Tre-292134 | 232115-232134 | AGGGTAAGGCGAAACTGGGT | 1309 |
| | | Tre-232198 | 232173-232198 | GAGAGATTCCGAAAGTAGTGGCGAGC | 1310 |
| | | Tre-232210 | 232182-232210 | CGAAAGTAGTGGCGAGCGAAATTGGAGGA | 1311 |
| | | Tre-232297 | 232270-232297 | AATAATCCGGCCTATAGCAGAAAGGTTT | 1312 |
| | | Tre-232329 | 232301-232329 | GAAAGCCTGACAGAGAGGGTGAAATCCCC | 1313 |
| | | Tre-232335 | 232312-232335 | AGAGAGGGTGAAATCCCCGTATGC | 1314 |
| | | Tre-232342 | 232319-232342 | GTGAAATCCCCGTATGCGGAATGG | 1315 |
| | | Tre-232357 | 232338-232357 | AATGGGGCGGACCTGCTGGT | 1316 |
| | | Tre-232407 | 232379-232407 | ACACGAGGAATCCTGTCGGAATCTGGGTC | 1317 |
| | | Tre-232421 | 232397-232421 | GAATCTGGGTCGACCACGATCTAAG | 1318 |
| | | Tre-232427 | 232405-232427 | GTCGACCACGATCTAAGGCTAAA | 1319 |
| | | Tre-232491 | 232464-232491 | GGGAAAGATGAAAAGAACCCCGGTGAGG | 1320 |
| | | Tre-232525 | 232502-232525 | AGAACCTGAAACCGTAAACCAACA | 1321 |
| | | Tre-232540 | 232519-232540 | ACCAACAACATGTTACAGCCTG | 1322 |
| | | Tre-232348 | 232523-232548 | ACAAGATGTTACAGCCTGTGAGGGTG | 1323 |
| | | Tre-232568 | 232543-232568 | AGGGTGGTAGCGTGCCTTTTGTAGAA | 1324 |
| | | Tre-232643 | 232622-232643 | CGGAGGGAAACCGAGTCTTAAA | 1325 |
| | | Tre-232669 | 232649-232669 | GTGGTGAGTTGTACGTCGTAG | 1326 |
| | | Tre-232677 | 232648-232677 | CGTGGTGAGTTGTACGTCGTAGACCCGAAG | 1327 |
| | | Tre-232702 | 232673-232702 | CGAAGCCAGGGTGATCTAGTTATGAGCAGG | 1328 |
| | | Tre-232746 | 232720-232746 | CCTTGTGGAGGACCGAACTATAATCTG | 1329 |
| | | Tre-232774 | 232754-232774 | AGGTATGGATGACTTGTGACT | 1330 |
| | | Tre-232783 | 232758-232783 | ATGGATGACTTGTGACTAGGAGTGAA | 1331 |
| | | Tre-232792 | 232766-232792 | CTTGTGACTAGGAGTGAAAGGCTAAAC | 1332 |
| | | Tre-232804 | 232783-232804 | AAGGCTAAACAAACCTGGAGAT | 1333 |
| | | Tre-232817 | 232790-232817 | AACAAACCTGGAGATAGCTGGTTCTCCC | 1334 |
| | | Tre-232850 | 232831-232850 | GGGACACCCTTATACAAAAC | 1335 |
| | | Tre-232857 | 232837-232857 | GCCTTATACAAAACTGTCGGA | 1336 |
| | | Tre-232890 | 232870-232890 | GATGGGCTAGGGGGTTTCATC | 1337 |
| | | Tre-232896 | 232876-232896 | CTAGGGGGTTTCATCCCCTAC | 1338 |
| | | Tre-232915 | 232896-232915 | CCAAACCCAATCAAACTCTG | 1339 |
| | | Tre-232948 | 232924-232948 | CAGTCAACGTGTGGGAGTGAGACTG | 1340 |
| | | Tre-232966 | 232939-232966 | AGTGAGACTGCGTGCGACAAGGTTCGTA | 1341 |
| | | Tre-233032 | 233010-233032 | TACCGCTTGAGTGTGAAATGAAG | 1342 |
| | | Tre-233039 | 233018-233039 | GAGTGTGAAATGAAGTGTGGGT | 1343 |
| | | Tre-233049 | 233022-233049 | GTGAAATGAAGTGTGGGTACCTGGACAG | 1344 |
| | | Tre-233119 | 233099-233119 | CTCACTGGTCGAGTACGCATG | 1345 |
| | | Tre-233151 | 233124-233151 | GATAATGTATCGGGGCTAAGCGGTATAC | 1346 |
| | | Tre-233161 | 233136-233161 | GGGCTAAGCGGTATACCGAAGCTACG | 1347 |
| | | Tre-233168 | 233145-233168 | GGTATACCGAAGCTACGGGTCTTG | 1348 |
| | | Tre-233191 | 233165-233191 | CTTGCATTTTTGGTGCAAGGCGGTAGG | 1349 |
| | | Tre-233212 | 233186-233212 | GGTAGGGGAGCATTCCATGTACTGATG | 1350 |
| | | Tre-233252 | 233229-233252 | GAGTTCTGGAGGGGATGGAAGAGA | 1351 |
| | | Tre-233264 | 233235-223264 | TGGAGGGGATGGAAGAGAGAATGCAAGGTAT | 1352 |
| | | Tre-293390 | 233367-233390 | AGGGTCGTAGTCGATGGGAATCCG | 1353 |
| | | Tre-223397 | 233373-233397 | GTAGTCGATGGGAATCCGGTTTATA | 1354 |
| | | Tre-233403 | 233382-233403 | GGGAATCCGGTTTATATTCCGG | 1355 |
| | | Tre-233409 | 233387-233409 | TCCGGTTTATATTCCGGAACCTC | 1356 |
| | | Tre-233421 | 233394-233421 | TATATTCCGGAACCTCTTGCAATTTCGA | 1357 |
| | | Tre-233434 | 233405-233434 | ACCTCTTGCAATTTCGATGGCAGGACGCGT | 1358 |
| | | Tre-233466 | 233438-233465 | GTGAAGCCCGGCCAAAGATTGGTAGTTT | 1359 |
| | | Tre-233489 | 233464-233489 | TTTGGTCTAAGTATCCGAGCCGTTTT | 1360 |
| | | Tre-233518 | 233489-233518 | TAAGAGCGATAGGCAAATCCGTCGTTCGAG | 1361 |
| | | Tre-233554 | 233535-233554 | ACTGGAGCGATGAGCGAAGG | 1362 |
| | | Tre-233566 | 233544-233566 | ATGAGCGAAGGGAAGCAGGTGTA | 1363 |
| | | Tre-233576 | 233549-233576 | CGAAGGGAAGCAGGTGTAGTCATGGCGA | 1364 |
| | | Tre-233609 | 233585-233609 | ACTGTCTAAGGTTAGGTTGCAAGAG | 1365 |
| | | Tre-233617 | 233592-233617 | AAGGTTAGGTTGCAAGAGACCGTACC | 1366 |
| | | Tre-233624 | 233601-233624 | TTGCAAGAGACCGTACCGCAAACC | 1367 |
| | | Tre-233678 | 233658-233678 | CTCGAGAGAACTCGCGTCAAG | 1368 |
| | | Tre-233685 | 233661-233685 | GAGAGAACTCGCGTCAAGGAACTCG | 1369 |
| | | Tre-233694 | 233671-233694 | GCGTCAAGGAACTCGGCAAAATAC | 1370 |
| | | Tre-233704 | 233677-233704 | AGGAACTCGGCAAAATACACACGTAACC | 1371 |
| | | Tre-233724 | 233705-233724 | TCGGGAGAAGTGTGACCCTT | 1372 |
| | | Tre-233736 | 233709-233736 | GAGAAGTGTGACCCTTGCCTTTGGTGAG | 1373 |
| | | Tre-233748 | 233727-233748 | CTTTGGTGAGGGTGGCAGAAAG | 1374 |
| | | Tre-233773 | 233749-233773 | CAGGTCCAGGCGACTGTTTATCAAA | 1375 |
| | | Tre-233795 | 233776-233795 | CATAGCCATCTGCAAATCAG | 1376 |
| | | Tre-233814 | 233795-233814 | GTAATGAGACGTATAGGTGG | 1377 |
| | | Tre-233825 | 233797-233825 | AATGAGACGTATAGGTGGTGACACCTGCC | 1378 |
| | | Tre-233888 | 233864-233888 | CAACGCTTTGAATTGAAGCCCCAGT | 1379 |
| | | Tre-234010 | 233988-234010 | GACGCGAGACTCGGTGAAATTTA | 1380 |
| | | Tre-234018 | 233999-234018 | CGGTGAAATTTATGTACCGG | 1381 |
| | | Tre-234062 | 234033-234062 | ACCCATAGTTAGACGGAAAGACCCCGTGAA | 1382 |
| | | Tre-234069 | 234047-234069 | GGAAAGACCCGTGAACCTTCAC | 1383 |
| | | Tre-234106 | 234084-234106 | GGAACTTGGTTTACCATGTGTAG | 1384 |
| | | Tre-234138 | 234117-234138 | AGACAGAGAAGCTTGGCCGTCA | 1385 |
| | | Tre-234144 | 234123-234144 | AGAAGCTTGGCCGTCAGGTTAG | 1386 |
| | | Tre-234171 | 234150-234171 | GTCAACAGTGAAATACCACCCT | 1387 |
| | | Tre-234182 | 234154-234182 | ACAGTGAAATACCACCCTTGGTACGTCAG | 1388 |
| | | Tre-234193 | 234166-234193 | CACCCTTGGTACGCAGGTTTCTAACCT | 1389 |
| | | Tre-234298 | 234279-234298 | AAGGTCTCCTCACACCGGTT | 1390 |
| | | Tre-234309 | 234288-234309 | TCTCCTCACACCGGTTGGAAATCGGTG | 1391 |
| | | Tre-234322 | 234297-234322 | TTGGAAATCGGTGCGCGAGTGTAAAG | 1392 |
| | | Tre-234355 | 234331-234355 | AGGCTTAACTGCGAGACCGACAGGT | 1393 |
| | | Tre-234365 | 234346-234365 | ACCGACAGGTCGAGCAGATA | 1394 |
| | | Tre-234541 | 234520-234541 | TGAAGCAGGTCCCAAGGGTTTG | 1395 |
| | | Tre-234800 | 234780-234800 | CCCTGAAGGTTGACCTTCCTG | 1396 |
| | Ureaplasma | Urea-147207 | 147178-147207 | CCTTGGGACAAACAGGCGATGAAGGACGTG | 1397 |
| | | Urea-147259 | 147240-147259 | GAGGCTTTAATCCGTTGATC | 1398 |
| | | Urea-147263 | 147242-147263 | GGCTTTAATCCGTTGATCTCCG | 1399 |
| | | Urea-147272 | 147248-147272 | AATCCGTTGATCTCCGAATGAGGAA | 1400 |
| | | Urea-143280 | 147255-147280 | TGATCTCCGAATGAGGAAACTCAATC | 1401 |
| | | Urea-147389 | 147370-147389 | GAAAACGAAGTGATTCCCTG | 1402 |
| | | Urea-147397 | 147372-147397 | AAACGAAGTGATTCCCTGTGTAGCGG | 1403 |
| | | Urea-147443 | 147414-147443 | AGGCCAAACCGAATTTCGATTCGGGGTTGT | 1404 |
| | | Urea-147455 | 147426-147455 | ATTTCGATTCGGGGTTGTAGGACTACAATA | 1405 |
| | | Urea-147501 | 147478-147501 | ATTGGTTGGGAAGCCAAATCATAG | 1406 |
| | | Urea-147509 | 147484-147509 | TGGGAAGCCAAATCATAGAGGGTGAT | 1407 |
| | | Urea-147523 | 147496-147523 | TCATAGAGGGTGATAATCCCGTATACGA | 1408 |
| | | Urea-147559 | 147534-147559 | TTACCTAGTAGGATCCTGAGTAGGGC | 1409 |
| | | Urea-147586 | 147566-147586 | CGTGAAATCCTGTCTGAATCC | 1410 |
| | | Urea-147636 | 147814-147636 | CTAGTTTGTCACCGATAGAGCAT | 1411 |
| | | Urea-147692 | 147670-147692 | GATGGGAGTGAAATAGAACCTGA | 1412 |
| | | Urea-147721 | 147700-147721 | AGCTTACAAGGTGTTAGAGCAC | 1413 |
| | | Urea-147751 | 147723-147751 | TTAATGTGTGATAGCGTGCCTTTTGAAGT | 1414 |
| | | Urea-147779 | 147754-147779 | GAGCCAGCGAGTTATTATAGCATGCG | 1415 |
| | | Urea-147796 | 147771-147796 | TAGCATGCGAGGTTAAATCGTAGAGA | 1416 |
| | | Urea-147807 | 147784-147807 | TAAATCGTAGAGATGGAGCCGTAG | 1417 |
| | | Urea-147813 | 147791-147813 | TAGAGATGGAGCCGTAGGGAAAC | 1418 |
| | | Urea-147866 | 147842-147866 | TATAATAGACGCGAAACGGGGTGAT | 1419 |
| | | Urea-147876 | 147850-147876 | ACGCGAAACGGGGTGATCTATCCATGG | 1420 |
| | | Urea-147889 | 147860-147889 | GGGTGATCTATCCATGGGCAGGTTGAAGGT | 1421 |
| | | Urea-147897 | 147872-147397 | CATGGGCAGGTTGAAGGTGAAGTAAC | 1422 |
| | | Urea-147923 | 147904-147923 | TGGAGGACCGAACCCACTTT | 1423 |
| | | Urea-147979 | 147960-147979 | GAAATTCCAATCGAACTCCG | 1424 |
| | | Urea-148041 | 148022-148041 | GATTATTGGGAATATGGGGG | 1425 |
| | | Urea-148072 | 148045-148072 | AGCACTGAATCTATGATGGCGCCACCTC | 1426 |
| | | Urea-148088 | 148065-148088 | GCCACCTCGGTGTACTGAATAGAA | 1427 |
| | | Urea-148135 | 148110-148135 | ACCTATTCTAGCAGTCAGACAGTGGG | 1428 |
| | | Urea-148184 | 148165-148184 | GCCCAGATCATTAACTAAGG | 1429 |
| | | Urea-148277 | 148258-148277 | CCGTTTAAAGAGTGCGTAAC | 1430 |
| | | Urea-148316 | 148291-148316 | AGAGACTCTGCGCGGAAGATGTAACG | 1431 |
| | | Urea-148330 | 148303-148330 | CGGAAGATGTAACGGGGCTAAGCATATA | 1432 |
| | | Urea-148337 | 148313-148337 | AACGGGGCTAAGCATATAACCGAAG | 1433 |
| | | Urea-148383 | 148355-148383 | TATATGTAGCGGTAGACGAGTGTTGTATA | 1434 |
| | | Urea-148409 | 148386-148409 | GGGCGAAGGTAGACTGTGAAGACT | 1435 |
| | | Urea-148418 | 148392-148418 | AGGTAGACTGTGAAGACTACTGGACTT | 1436 |
| | | Urea-148461 | 148422-148461 | GAGTAACGAATGAGAGTGAG | 1437 |
| | | Urea-148467 | 148448-148467 | CGAATGAGAGTGAGAATCTC | 1438 |
| | | Urea-148499 | 148471-148499 | AACCGATTGACTAAGGGTTCCTGGGCAAG | 1439 |
| | | Urea-148536 | 148510-143536 | CCAGGGTAAGTCGGATCCTAAGGCGAG | 1440 |
| | | Urea-148549 | 148523-148549 | GATCCTAAGGCGAGGCTGAAAAGCGTA | 1441 |
| | | Urea-148627 | 148604-148627 | CGGAGAAGGTTATTATGTGCCGGT | 1442 |
| | | Urea-148643 | 148618-148643 | ATGTGCCGGTTATTGGATTCCGGTTT | 1443 |
| | | Urea-148677 | 148656-148677 | TAGTAAGTTGGCAAATCCGCTT | 1444 |
| | | Urea-148681 | 148660-148681 | AAGTTGCCAAATCCGCTTACTA | 1445 |
| | | Urea-148707 | 148688-148707 | CCAAGTTATGAATACGAGCG | 1446 |
| | | Urea-148715 | 148691-148715 | AGTTATGAATACGAGCGACCCCTTC | 1447 |
| | | Urea-148737 | 148713-148737 | TTCGGGCAGTAGCGAAGACATATAC | 1448 |
| | | Urea-148766 | 148746-148766 | TCCAAGAAAAGCTTCTAGCGT | 1449 |
| | | Urea-148805 | 148777-148805 | GTAGTCCGTACCGAGAACGAACACACGTG | 1450 |
| | | Urea-148839 | 148820-148839 | CCTAAGGTTAGCGAGTTAAC | 1451 |
| | | Urea-148844 | 148825-148844 | GGTTAGCGAGTTAACTACAG | 1452 |
| | | Urea-148858 | 148839-148858 | CTACAGTTAAGGAACTCTGC | 1453 |
| | | Urea-148886 | 148849-148876 | GGAACTCTGCAAATTAACCCCGTACGTT | 1454 |
| | | Urea-148903 | 148879-148903 | CAATAAGGGGTGCTCGCTGTAAAAG | 1455 |
| | | Urea-148911 | 148886-148911 | GGGTGCTCGCTGTAAAAGGTGAGCCG | 1456 |
| | | Urea-148939 | 148917-148939 | AATAGCGAGGGGGGACTGTTTAA | 1457 |
| | | Urea-148972 | 148953-148972 | CTATGCTAAGTCGTAAGACG | 1458 |
| | | Urea-148984 | 148964-148984 | CGTAAGACGATGTATATGGGG | 1459 |
| | | Urea-148995 | 148967-148995 | AAGACGATGTATATGGGGTGACACCTGGC | 1460 |
| | | Urea-149012 | 148985-149012 | TGACACCTGCCCAATGCTGTAAGGTTAA | 1461 |
| | | Urea-149079 | 149054-143079 | AGTGAATGGCGGCCGTAACTATAACG | 1462 |
| | | Urea-149157 | 149135-149157 | AACCATCTCTTGACTGTCTCAAC | 1463 |
| | | Urea-149173 | 149145-149173 | TGACTGTCTCAACTGTAGACTCGGTGAAA | 1464 |
| | | Urea-149184 | 149156-149184 | ACTGTAGACTCGGTGAAATCCTGGTGAGG | 1465 |
| | | Urea-149192 | 149167-149192 | GGTGAAATCCTGGTGAGGGTGAAGAC | 1466 |
| | | Urea-149304 | 149282-149304 | GGAGACTGTGAAGTATACTCGCT | 1467 |
| | | Urea-149310 | 149287-149310 | CTGTGAAGTATACTCGCTAGGGTA | 1468 |
| | | Urea-149338 | 149313-149338 | TGGAGTCAACGTTGGAATACTACCCT | 1469 |
| | | Urea-149342 | 149321-149342 | ACGTTGGAATACTACCCTTGTG | 1470 |
| | | Urea-149386 | 149367-149386 | ATGAATCTGGCTGGGGGACA | 1471 |
| | | Urea-149531 | 149510-149531 | GAGACTTACAAGTCGAACAGGT | 1472 |
| | | Urea-149368 | 149542-149568 | GTCATAGTGATCCGGTGGCTCAGAATG | 1473 |
| | | Urea-149670 | 149644-149670 | GCACTGTTTGGGACCTCGATGTCGACT | 1474 |
| | Vibrio | Vib-2331 | 2311-2331 | TAGTGCAATCGCATAAGCCCG | 1475 |
| | | Vib-1795 | 1776-1795 | CAGAGCACTGTGCAAAATCG | 1476 |
| | | Vib-1608 | 1585-1608 | GGAATCGTACCCCAAACCGACACA | 1477 |
| | | Vib-1561 | 1536-1561 | GTCATTGATGCCATGCTTCCAGGAAA | 1478 |
| | Yersinia | Yer-332 | 313-332 | TCGCACGGTACAGGGTGATA | 1479 |
| | | Yer-336 | 315-336 | GCACGGTACAGGGTGATAGTCC | 1480 |
| | | Yer-626 | 598-626 | AGCAAGGTTAACCGAATAGGGGAGCCGTA | 1481 |
| | | Yer-1231 | 1206-1231 | CCGTTGAAGGTGACCTGTGAGGGTTG | 1482 |
| | | Yer-1509 | 1490-1509 | GGTAAATCCGGTTGCTTATC | 1483 |
| | | Yer-2210 | 2188-2210 | GTTTGATGTTCTAACTCGGCCCC | 1484 |

Target regions of standard strains: Acinetobacter (GenBank Accession No.: X87280), Actinomyces (temporary SEQ NO: 2), Aeromonas (GenBank Accession No.: AF508056), Bacillus (GenBank Accession No.: D11459), Bacteroides (GenBank Accession No.: NC_{—}004663), Bordetella (GenBank Accession No.: X68323), Borrelia (GenBank Accession No.: NC_{—}001318), Brucella (GenBank Accession No.: NC_{—}004311), Burkholderia (GenBank Accession No.: Y17182), Campylobacter (GenBank Accession No.: U09611), Chlamydia (GenBank Accession No.: NC_{—}000117), Citrobacter (GenBank Accession No.: U77928), Clostridium (GenBank Accession No.: M94260), Corynebacterium (GenBank Accession No.: NC_{—}004369), Enterbacter (temporary SEQ NO: 6), Enterococcus (GenBank Accession No.: AJ295298), Fusobacterium (GenBank Accession No.: AJ307974), Haemophilus (GenBank Accession No.: NC_{—}002940), Helicobacter (GenBank Accession No. AB088050), Klebsiella (temporary SEQ NO: 10), Legionella (temporary SEQ NO: 12), Listeria (GenBank Accession No.: X92948), Morganella (temporary SEQ NO: 13), Mycobacteria (GenBank Accession No.: Z17212), Mycoplasma (GenBank Accession No.: X68422), Neisseria (GenBank Accession No.: NC_{—}003112), Peptococcus (GenBank Accession No.: X68428), Plesiomonas (GenBank Accession No.: X65487), Porphyromonas (GenBank Accession No.: NC_{—}002950), Propionibacterium (temporary SEQ NO: 29), Providencia (temporary SEQ NO: 30), Pseudomonas (GenBank Accession No.: Y00432), Salmonella (GenBank Accession No.: U77921), Shigella (GenBank Accession No.: NC_{—}004741), Staphylococcus (GenBank Accession No.: X68425), Streptococcus (GenBank Accession No.: AB096740), Treponema (GenBank Accession No.: NC_{—}000919), Ureaplasma (GenBank Accession No.: NC_{—}002162), Vibrio (GenBank Accession No.: AJ310649), Yersinia (GenBank Accession No.: U77925) are referred for the nucleotide sequence analysis.

### EXAMPLES

Practical and presently preferred embodiments of the present invention are illustrated as shown in the following Examples.

However, it will be appreciated that those skilled in the art, on consideration of this disclosure, may make modifications and improvements within the spirit and scope of the present invention.

### Example 1

### Cell Culture and Separation of Genome DNA

Approximately 100 kinds of microbial strains were purchased from American Type Culture Collection (ATCC, U.S.A) and Korean Collection for Type Cultures (KCTC, Korea). In order to cultivate each microbe, culture medium and condition were adjusted according to the manual recommended by ATCC and KCTC. Cell colonies were collected and injected into 1.5 ml tube. Then, 100 μl of InstaGene matrix (purchased from Bio-Rad, USA) was added and reacted with a water bath at 56° C. for 30 minutes. After stirring for 10 seconds, the resulting cells were heat-treated, stirred again for 10 minutes and centrifuged for 3 minutes at 12,000 rpm to collect a cell supernatant. For negative control groups, tertiary distilled water (referred to as “N” in FIGs), human DNA and viral DNA were utilized to standardize the amplification in following Examples.

Experimental strains used to analyze nucleotide sequences are summarized as follows.

TABLE 4

| | | | ATCC |
|---|---|---|---|
| No. | Genus Name | Strain Name | No. |
| 1-1 | Acinetobacter | Acinetobacter baumannii | 19606 |
| 2-1 | Actinomyces | Actinomyces bovis | 13683 |
| 2-2 | | Actinomyces israeii | 12104 |
| 2-3 | | Actinomyces viscosus | 15988 |
| 3-1 | Aeromonas | Aeromonas hydrophila | 7966 |
| 3-2 | | Aeromonas salmonicida | 33658 |
| 4-1 | Bacillus | Bacillus cereus | 11950 |
| 5-1 | Bacteroides | Bacteroides fragilis | 25285 |
| 5-2 | | Bacteroides thetaiotaomicron | 29741 |
| 5-3 | | Bacteroides forsythus | 25577 |
| 5-4 | | Bacteroides ureolyticus | 33387 |
| 6-1 | Bordetella | Bordetella pertussis | 10380 |
| 7-1 | Borrelia | Borrelia burgdorferi | 35210 |
| 8-1 | Burkholderia | Burkholderia cepacia | 25416 |
| 9-1 | Campylobacter | Campylobacter coli | 33559 |
| 9-2 | | Campylobacter fetus | 15296 |
| 9-3 | | Campylobacter jejuni | 29428 |
| 9-4 | | Campylobacter rectus | 33238 |
| 10-1 | Citrobacter | Citrobacter freundii | 33128 |
| 11-1 | Clostridium | Clostridium difficile | 17857 |
| 11-2 | | Clostridium perfringens | 25768 |
| 11-3 | | Clostridium septicum | 11424 |
| 11-4 | | Clostridium tetani | 10709 |
| 12-1 | Corynebacterium | Corynebacterium diptheriae | 11913 |
| 13-1 | Enterobacter | Enterobacter aerogenes | 13048 |
| 13-2 | | Enterobacter agglomerans | 27155 |
| 13-3 | | Enterobacter cloacae | 13047 |
| 14-1 | Enterococcus | Enterococcus avium | 14025 |
| 14-2 | | Enterococcus faecalis | 19433 |
| 14-3 | | Enterococcus faecium | 8043 |
| 14-4 | | Enterococcus hirde | 10541 |
| 15-1 | Escherichia | Escherichia coli | 10536 |
| 16-1 | Eubacterium | Eubacterium limosum | 10825 |
| 17-1 | Fusobacterium | Fusobacterium mortiferum | 25557 |
| 17-2 | | Fusobacterium necroforum | 25286 |
| 17-3 | | Fusobacterium nucleatum | 25586 |
| 17-4 | | Fusobacterium prausnitzii | 27766 |
| 18-1 | Haemophilus | Haemophilus influenzae | 10211 |
| 18-2 | | Haemophilus parainfluenzae | 33392 |
| 19-1 | Helicobacter | Helicobacter pylori | 43504 |
| 20-1 | Klebsiella | Klebsiella oxytoca | 13182 |
| 20-2 | | Klebsiella pneumoniae | 15380 |
| 21-1 | Legionella | Legionella pneumophilia | 33152 |
| 22-1 | Listeria | Listeria monocytogenes | 19115 |
| 23-1 | Morganella | Morganella morganii | 25830 |
| 24-1 | Mycobacteria | Mycobacterium tuberculosis | 27294 |
| 24-2 | | Mycobacterium avium | 25291 |
| 24-3 | | Mycobacterium intracellulare | 13950 |
| 24-4 | | Mycobacterium fortuitum | 6841 |
| 24-5 | | Mycobacterium chelonae | 35752 |
| 24-6 | | Mycobacterium abscessus | 19977 |
| 24-7 | | Mycobacterium kansasii | 12478 |
| 24-8 | | Mycobacterium gordonae | 14470 |
| 24-9 | | Mycobacterium scrofulace | 19981 |
| 24-10 | | Mycobacterium szulgai | 35799 |
| 24-11 | | Mycobacterium vacoae | 15483 |
| 24-12 | | Mycobacterium xenopi | 19250 |
| 24-13 | | Mycobacterium terrae | 15755 |
| 24-14 | | Mycobacterium flavescence | 14474 |
| 24-15 | | Mycobacterium smegmatis | 21701 |
| 24-16 | | Mycobacterium malmoense | 29571 |
| 24-17 | | Mycobacterium simiae | 25275 |
| 24-18 | | Mycobacterium marinum | 927 |
| 24-19 | | Mycobacterium ulcerans | 19423 |
| 24-20 | | Mycobacterium gastri | 15754 |
| 25-1 | Mycoplasma | Mycoplasma arginini | 23838 |
| 25-2 | | Mycoplasma fermentans | 19989 |
| 25-3 | | Mycoplasma orale | 23714 |
| 25-4 | | Mycoplasma hyorhinis | 17981 |
| 25-5 | | Mycoplasma hominis | 23114 |
| 25-6 | | Mycoplasma salivarium | 23064 |
| 25-7 | | Mycoplasma pirum | 25960 |
| 25-8 | | Mycoplasma arthritidis | 19611 |
| 25-9 | | Mycoplasma cloacale | 35276 |
| 25-10 | | Mycoplasma falconis | 51372 |
| 25-11 | | Mycoplasma genitalium | 33530 |
| 25-12 | | Mycoplasma hyosynoviae | 25591 |
| 25-13 | | Mycoplasma bovis | 27368 |
| 25-14 | | Mycoplasma muris | 33757 |
| 25-15 | | Mycoplasma neurolyticum | 19988 |
| 25-16 | | Mycoplasma opalescens | 27921 |
| 25-17 | | Mycoplasma penetrans | 55252 |
| 25-18 | | Mycoplasma pneumoniae | 15531 |
| 25-19 | | Mycoplasma primatum | 15497 |
| 25-20 | | Mycoplasma pulmonis | 14267 |
| 25-21 | | Mycoplasma spermatophilum | 49695 |
| 25-22 | | Mycoplasma synoviae | 25204 |
| 25-23 | | Mycoplasma faucium | 25293 |
| 26-1 | Neisseria | Neisseria gonorrheae | 19424 |
| 26-2 | | Neisseria meningitidis | 13077 |
| 27-1 | Peptococcus | Peptococcus magnus | 29328 |
| 28-1 | Peptostreptococcus | Peptostreptococcus | 14963 |
| | | asaccharolyticus | |
| 28-2 | | Peptostreptococcus prevotii | 9321 |
| 29-1 | Plesiomonas | Plesiomonas shigelloides | 14029 |
| 30-1 | Porphyromonas | Porphyromonas asaccharolytica | 25260 |
| 30-2 | | Porphyromonas gingivalis | 33277 |
| 31-1 | Prevotella | Prevotella intermedia-corporis | 25611 |
| 31-2 | | Prevotella melaninogenica | 25845 |
| 31-3 | | Prevotella nigrescens | 25261 |
| 31-4 | | Prevotella oralis | 33269 |
| 32-1 | Propionibacterium | Propionibacterium acnes | 6919 |
| 32-2 | | Propionibacterium avium | 25577 |
| 32-3 | | Propionibacterium granulosum | 25564 |
| 33-1 | Proteus | Proteus mirabilis | 29906 |
| 33-2 | | Proteus vulgaris | 13315 |
| 34-1 | Providencia | Providencia stuarti | 29914 |
| 35-1 | Pseudomonas | Pseudomonas aeruginosa | 10145 |
| 36-1 | Salmonella | Salmonella bongori | 43975 |
| 36-2 | | Salmonella enteritidis | 4931 |
| 37-1 | Serratia | Serratia marcescens | 13880 |
| 38-1 | Shigella | Shigella boydii | 8700 |
| 38-2 | | Shigella dysenteriae | 13313 |
| 38-3 | | Shigella flexneri | 9199 |
| 38-4 | | Shigella sonnei | 25931 |
| 39-1 | Staphylococcus | Staphylococcus aureus | 25923 |
| 39-2 | | Staphylococcus epidermidis | 12228 |
| 39-3 | | Staphylococcus saccharolyticus | 14953 |
| 39-4 | | Staphylococcus saprophyticus | 15305 |
| 40-1 | Stenotrophomonas | Stenotrophomonas maltophilia | 13637 |
| 41-1 | Streptococcus | Streptococcus agalactiae | 13813 |
| 41-2 | | Streptococcus anginosus | 31412 |
| 41-3 | | Streptococcus bovis | 33317 |
| 41-4 | | Streptococcus constellatus | 27513 |
| 41-5 | | Streptococcus intermedius | 27335 |
| 41-6 | | Streptococcus pneumoniae | 33400 |
| 41-7 | | Streptococcus pyogenes | 19615 |
| 42-1 | Ureaplasma | Ureaplasma urealyticum | 27618 |
| 43-3 | Veilonella | Veilonella parrula | 10790 |
| 44-1 | Vibrio | Vibrio cholerae | 12022 |
| 44-2 | | Vibrio parahemolyticus | 17802 |
| 44-3 | | Vibrio vulnificus | 27562 |
| 45-1 | Yersinia | Yersinia enterocolitica | 9610 |
| 45-2 | | Yersinia pseudotuberculosis | 29833 |

### Example 2

### Construction of Primers for Microbial Diagnosis

### 1. Design of Bacterial-Specific Primers for Diagnosis of Microorganism

The primers of the present invention for detecting the presence of microorganism were designed on a basis of the multiple alignment and BLAST analysis in 23S rDNA nucleotide sequences of bacterium. The nucleotide sequence having the high homology with that of target microbe, but the low homology with those of other microorganism was determined to design primers of Table 2 corresponding to temporary SEQ NO: 38˜SEQ ID NO: 135. The bacterial-specific primers of the present invention are not limited within the nucleotide sequences of Table 2, but may be modified. Any probe containing the nucleotide sequences if not influencing the property can be designed.

### 2. Design of Bacterial Species-Specific Primers for Diagnosis of Microorganism

The species-specific primers of the present invention are not limited within the nucleotide sequences of Table 3, but may be modified. Any probe containing the nucleotide sequences if not influencing the property can be designed.

### (1) Construction of Specific Primers for Detection of Aeromonas Sp.

In order to amplify a target gene specific for all strains of Aeromonas sp., the 23S rDNA gene was adopted. The nucleotide sequence specific for Aeromonas sp. and having less sequence homology with other microorganism was determined to design primers of Table 3 corresponding to temporary SEQ NO: 197˜SEQ ID NO: 216.

### (2) Construction of Specific Primers for Detection of Enterococcus Sp.

In order to amplify a target gene specific for all strains of Enterococcus sp., the 23S rDNA gene was adopted. The nucleotide sequence specific for Enterococcus sp. and having less sequence homology with other microorganism was determined to design primers of Table 3 corresponding to temporary SEQ NO: 699˜SEQ ID NO: 703.

### (3) Construction of Specific Primers for Detection of Mycobacteria Sp.

In order to amplify a target gene specific for all strains of Mycobacteria sp., the 23S rDNA gene was selected. The nucleotide sequence specific for Mycobacteria sp. and having less sequence homology with other microorganism was determined to design primers of Table 3 corresponding to temporary SEQ NO: 872˜SEQ ID NO: 880.

### (4) Construction of Specific Primers for Detection of Streptococcus Sp.

In order to amplify a target gene specific for all strains of Streptococcus sp., the 23S rDNA gene was adopted. The nucleotide sequence specific for Streptococcus sp. and having less sequence homology with other microorganism was determined to design primers of Table 3 corresponding to temporary SEQ NO: 1287˜SEQ ID NO: 1298.

### Example 3

### Amplification of Target DNAs

In order to detect the presence of microorganism and identify each species of pathogen, DNA primers for the amplification were prepared as follows.

TABLE 5

| | | Temp. |
|---|---|---|
| Strain Name | Primer | Seq. No. |
| Bacteria | 389R | 42 |
| | 459R | 46 |
| | 469R | 48 |
| | 471R | 49 |
| | 520R | 54 |
| | 991R | 64 |
| | 1075R | 70 |
| | 1906R | 90 |
| | 1920R | 91 |
| | 1941R | 93 |
| | 1961R | 94 |
| | 2069R | 99 |
| | 2252R | 105 |
| | 2431R | 115 |
| | 2443R | 117 |
| | 2504R | 120 |
| | 2517R | 122 |
| | 2607R | 132 |
| Genus Aeromonas | Aer-665F | 199 |
| | Aer-1417R | 207 |
| Genus Enterococcus | Entc-310F | 699 |
| | Entc-909R | 701 |
| Geuus Mycobacteria | MB-2089F | 875 |
| | MB-3051R | 880 |
| Genus Streptococcus | Str-791F | 1289 |
| | Str-1595R | 1291 |

16S-1387F primer: primers for the detection of general microorganism designed on basis of 16S rDNA sequence already determined (Applied and Environmental Microbiology, 64(2): 795˜799, 1998).

The above-mentioned sets of primers were utilized to perform a PCR method in each genomic DNA of standard strain separated through the same procedure described in Example 1.

### (1) Preparation of PCR Mixture (25 μl of Final Volume)

PCR mixture was prepared as follows: 100 mM KCl, 20 mM Tris HCl (pH 9.0), 1% Triton X-100, 10 mM deoxynucleoside triphosphates (dATP, dGTP, dTTP, and dCTP), 1.5 mM MgCl₂, A pair of primers (10 pmole respectively), 1 U Taq polymerase (QIAGEN, USA), and 4 μl of template DNA.

### (2) PCR Condition

The reaction mixture was denatured for 3 minutes at 94° C. sufficiently, amplified at 94° C. for 1 minute, at 55° C. for one and a half minute and 72° C. for 2 minutes and finally, extended at 72° C. for 10 minutes.

### Example 4

### Examination of Amplified Products

PCR products amplified through the procedure described in Example 3 were analyzed by performing A gel electrophoresis.

FIG. 4 depicts the PCR result by using a pair of primers amplifying the 23S rDNA target sequence for the bacterial-specific detection. FIG. 4 illustrates the PCR products in approximately 800˜2,500 bp that is amplified with the forward primer 16S-1387F designed by using the 16S rDNA gene and the reverse primer (temporary SEQ NO: 42, 46, 48, 49, 54, 64, 70, 90, 91, 93, 94, 99, 105, 115, 117, 120, 122 or 132) designed by using the 23S rDNA gene of the present invention in a pair and analyzed by performing a gel electrophoresis. In FIG. 4(a)˜FIG. 4(r), lane M is 100 bp Plus DNA ladder as a standard marker of molecular weight; lane N, a negative control group; lane 1˜10 are bacteria: respectively, lane 1 is the PCR product of Acinetobacter baumannii; lane 2, Aeromonas salmonicida; lane 3, Bacteroides forsythus; lane 4, Clostridium difficile; lane 5, Legionella pneumophilia; lane 6, Morganella morganii; lane 7, Porphyromonas asaccharolytica; lane 8, Proteus mirabilis; lane 9, Mycobacterium tuberculosis; and lane 10, Mycoplasma pneumoniae. As a result, it is clarified that the bacterial-specific PCR product are amplified by using each pair of specific primers, discriminating primarily other microorganism such as human DNA and viral DNA. This enables a rapid and precise diagnosis and reduces a diagnostic cost.

FIG. 6 depicts the PCR result by using a pair of primers amplifying the 23S rDNA target sequence for the bacterial genus-specific detection. FIG. 6a illustrates the 752 bp PCR product specific for Aeromonas that is amplified by using a pair of primers (temporary SEQ NO: 199 and SEQ ID NO: 207) and analyzed by performing a gel electrophoresis. FIG. 6b illustrates the 599 bp PCR product specific for Enterococcus that is amplified by using a pair of primers (temporary SEQ NO: 699 and SEQ ID NO: 701) and analyzed by performing a gel electrophoresis. FIG. 6c illustrates the 962 bp PCR product specific for Mycobacteria that is amplified by using a pair of primers (temporary SEQ NO: 875 and SEQ ID NO: 880) and analyzed by performing a gel electrophoresis. FIG. 6d illustrates the 804 bp PCR product specific for Streptococcus that is amplified by using a pair of primers (temporary SEQ NO: 1289 and SEQ ID NO: 1291) and analyzed by performing a gel electrophoresis. As a result, it is confirmed that the PCR products specific for each bacterial genus are amplified by using each pair of specific primers. This enables a rapid and precise diagnosis by identifying a bacterial genus to treat diseases properly, while reducing a diagnostic cost and preventing the abuse of antibiotics.

### Example 5

### Design of Probe for Differential Diagnosis of Bacteria

In order to design the probes of the present invention for detecting the presence of microorganism, the nucleotide sequences of 23S rDNA genes were first determined and analyzed. The probes of the present invention were designed on a basis of the multiple alignment in the 23S rDNA nucleotide sequences of bacteria selected from a group comprising Acinetobacter baumannii, Actinomyces bovis, Aeromonas salmonicida, Bacteroides ureolyticus, Clostridium difficile, Enterobacter aerogens, Enterococcus fecium, Eubacterium limocium, Fusobacterium moltiferum, Klebsiella ocitoca, Klebsiella pneumoniae, Legionella pneumophilia; Morganella morganii; Mycobacterium godone, Mycobacterium marinum, Mycobacterium xenopi, Mycobacterium flavescence, Mycobacterium scroflacium, Mycobacterium simiae, Mycobacterium suzukai, Mycobacterium pirum, Mycobacterium cloacole, Mycobacterium opalescence, Mycobacterium salibarium, Mycobacterium spulmatopi, Neisseria gonorohae, Peptococcus magnas, Propiobacterium evidum, Propiobacterium granulosium, Providencia stuati, Salmonella bongori, Shigella boidi, Shigella discentriae, Shigella sonnei, Staphylococcus chapropiticus, Streptococcus bovis and Yersinia pseudotuberculosis. The probes were designed to have the high homology to bacterial 23S rDNA genes by adopting conservative sequences. In detail, the probes contained the nucleotide sequences of temporary SEQ NO: 38˜SEQ ID NO: 135 as demonstrated in Table 2 and may hybridize 45 kinds of bacterial genera exclusively. The oligonucleotide probes of the present invention specific for bacteria, bacterial genera and bacterial species were synthesized to retain a dT spacer having 15 bases at the 5′-terminus and contain 15˜25 nucleotides. The bacterial-specific probes and the bacterial genus-specific probes in the present invention are not limited within the nucleotide sequences of Table 2 and Table 3, but may be modified. Any probe containing the nucleotide sequences if not influencing the property can be designed. In the present invention, 2 kinds of probes were utilized to conduct the bacterial species-specific detection: In detail, the nucleotide sequence of temporary SEQ NO: (TGCATGACAACAAAG) in Mycobacterium tuberculosis) and the nucleotide sequence of temporary SEQ NO: (GTAAATTAAACCCAAATCCC) in Mycoplasma pneumoniae were adopted.

### Example 6

### Preparation of Target DNA

### 1. Preparation of Target DNA Specific for Bacteria and Bacterial Genera and Species for Differential Diagnosis

In order to amplify target DNAs for the bacterial-specific and the bacterial genus-specific detection, the 23S rDNA gene were amplified in 689 bp and 701 bp of size selectively by using biotin-labeled primers: bio-389F (5′-biotin-TANGGCGGGACACGTGAAAT-3′) and bio-1075R (5′-biotin-GATGGCTGCTTCTAAGCCAAC-3′), and bio-1906F (5′-biotin-CCVGTAAACGGCGGCCG-3′) and bio-2607R (5′-biotin-GGACCGAACTGTCTCACGAC-3′) respectively. In order to perform the bacterial species-specific detection, the ITS region having approximately 700 bp of size was amplified by using the terminal region of 16S rDNA gene (16S-1387F) and the initial end region of 23S rDNA gene (temporary SEQ NO: 42). Each standard bacterial strain separated in Example 1 was examined by performing the PCR with the primers as follows: denaturing at 94° C. for 3 minutes under heat, then repeating to react at 94° C. for 1 minute, 50° C. for 1 minute, and 72° C. for 1 minute 35 times and finally extending at 72° C. for 10 minutes.

### Example 7

### Attachment of Probes onto a Substrate

Above all, one preferable kind of probes were selected in each bacterium, bacterial genus and bacterial species from the probes designed in Example 5, and diluted to 50 pmol by adding a spotting solution. The resulting probes were attached onto a slide glass substrate by using a microarray (Cartesian Technologies, PLXSYS 7500 SQXL Microarryer, USA). Then, the resulting microarray was placed in a slide box at a room temperature for 24 hours or incubated with a dry oven at 50° C. for about 5 hours to fix the probes.

### Example 8

### Washing of Unfixed Probes

In order to remove probes remained not to react onto the substrate, the microarray was washed out by using 0.2% SDS (sodium dodecyl sulfate) at a room temperature and then, washed by using distilled water. Again, the resulting microarray was washed out by using sodium borohydride, then washed out by using boiled distilled water and washed out again by using SDS and distilled water. Then, the surface of substrate was dried completely to finish up the preparation of microarrays.

### Example 9

### Labeling of Probes and Hybridization

In order to prepare single-stranded target DNAs, the biotin-labeled target DNAs prepared in Example 6 were denatured at more than 95° C. under heat and then, cooled at 4° C. In order to hybridize the PCR product and the probes, 10 μl of hybridization solution comprising a reactant solution containing Cy5-streptavidin or Cy3-streptavidin (Amersham Pharmacia biotech., USA) and 1˜5 μl of the target DNA was prepared. The hybridization solution was added to the slide completed to washed out after attaching probes. Then, the resulting slide was covered with a slide cover and reacted at 40° C. for 30 minutes.

### Example 10

### Washing of Unbound DNAs

In order to remove DNA remnants after the hybridization, the cover glass was put off and then, washed out by using by 2×SSC (300 mM NaCl, 30 mM Na-Citrate, pH 7.0) and 0.2×SSC buffer solution orderly. After that, the resulting slide was washed out to dried completely.

### Example 11

### Data Analysis

In order to analyze the experimental data, non-confocal laser scanner, GenePix 4000A (Axon Instruments, USA) was operated to estimate the results.

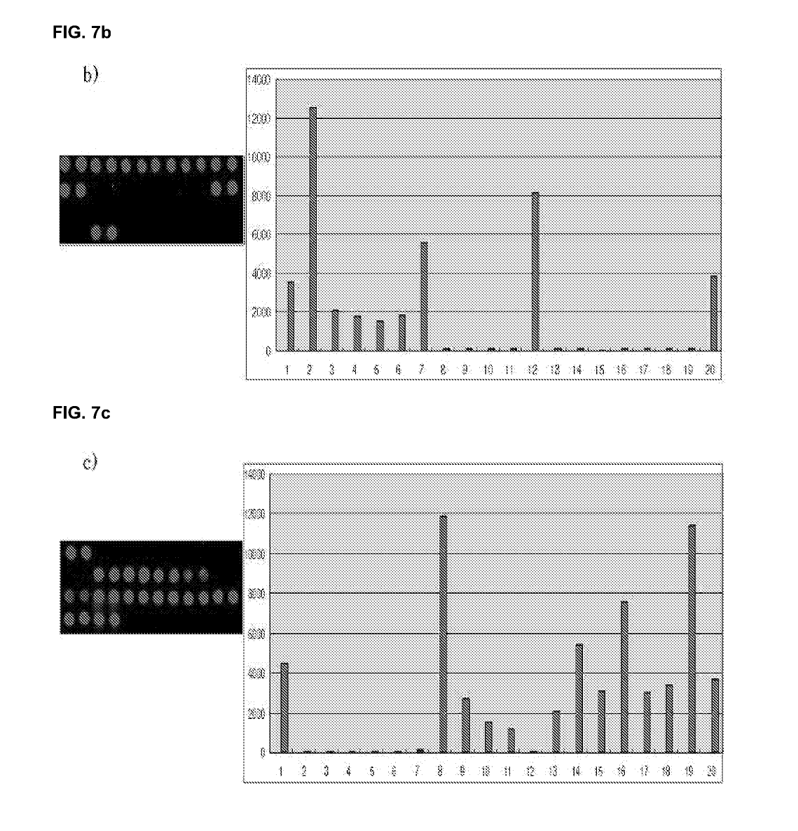

FIG. 7 to FIG. 9 depict the preferred embodiments of microarrays in the present invention. FIG. 7a illustrates the microarray comprising a substrate with one set of probes to detect the presence of microorganism: No. 2˜19 are the temporary SEQ NOS of the bacterial-specific probes in Table 2 (2; 42, 3; 46, 4; 48, 5; 49, 6; 54, 7; 64, 8; 90, 9; 91, 10; 93, 11; 94, 12; 70, 13; 99, 14; 105, 15; 115, 16; 117, 17; 120, 18; 122, 19; 132); No. 1 and 20 are positive probes (a mixture of all probes). FIG. 7b˜6c depict the result of hybridization by using each specific probe after performing the image analysis and estimating the intensity of its image elements. FIG. 7b illustrates the result that is amplified in approximately 680 bp from the initial end region of 23S rDNA gene by using bio-389F primer and bio-1075R primer in order to detect the presence of Mycobacterium tuberculosis, then hybridized with the bacterial-specific probes (the numbers of probes are denoted with temporary SEQ NOS: —2; 42, 3; 46, 4; 48, 5; 49, 6; 54, 7; 64, 12; 70) and analyzed resulting images to estimate the intensity of their image elements. FIG. 7c illustrates the result that is amplified in approximately 700 bp from the posterior end of 23S rDNA gene by using bio-1906F primer and bio-2607R primer in order to detect the presence of Streptococcus anginosus, then hybridized with the bacterial-specific probes (the numbers of probes are denoted with temporary SEQ NOS: —8; 90, 9; 91, 10; 93, 11; 94, 13; 99, 14; 105, 15; 115, 16; 117, 17; 120, 18; 122, 19; 132) and analyzed resulting images to estimate the intensity of its image elements. As a result, it is confirmed that the all bacterial-specific probes appear a positive signal, even if varied in the intensity of image elements.

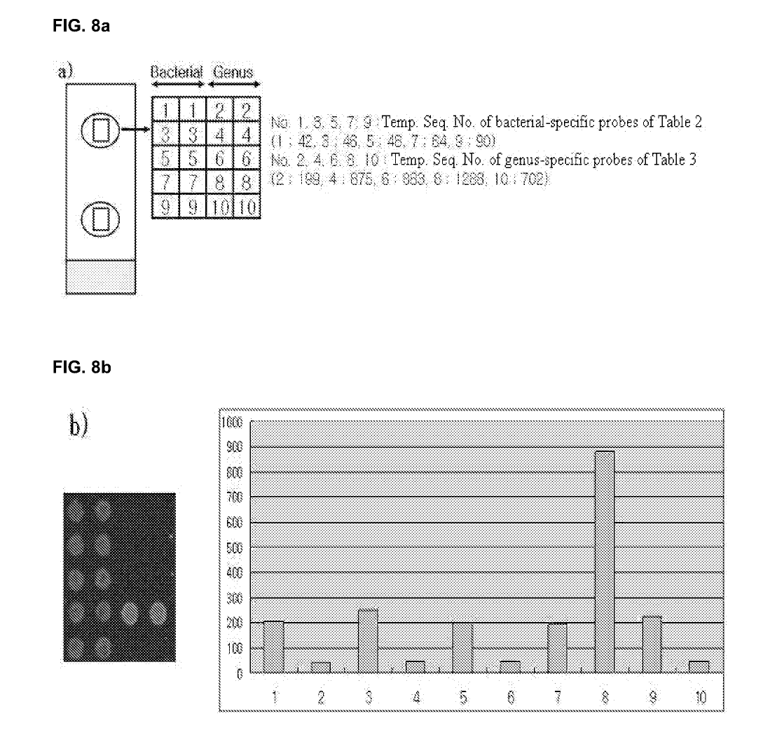

FIG. 8a depicts the microarray comprising a substrate with one set of probes to detect the presence of microorganism and identify a bacterial genus. No. 1, 3, 5, 7 and 9 are the temporary SEQ NOS of the bacterial-specific probes in Table 2 (1; 42, 3; 46, 5; 48, 7; 64, 9; 90) and No. 2, 4, 6, 8 and 10, the temporary SEQ NOS of the bacterial genus-specific probes in Table 3 (2; 199, 4; 875, 6; 883, 8; 1288, 10; 702). FIG. 8b depicts the result of hybridization by using the specific probes for Streptococcus sp. after performing the image analysis and estimating the intensity of its image elements. As a result, it is verified that the bacterial-specific probes, 1, 3, 5, 7 and 9 appear a positive signal and Streptococcus genus-specific probe 8 (temporary SEQ NO: 1288) appears a positive signal from the bacterial genus-specific probes

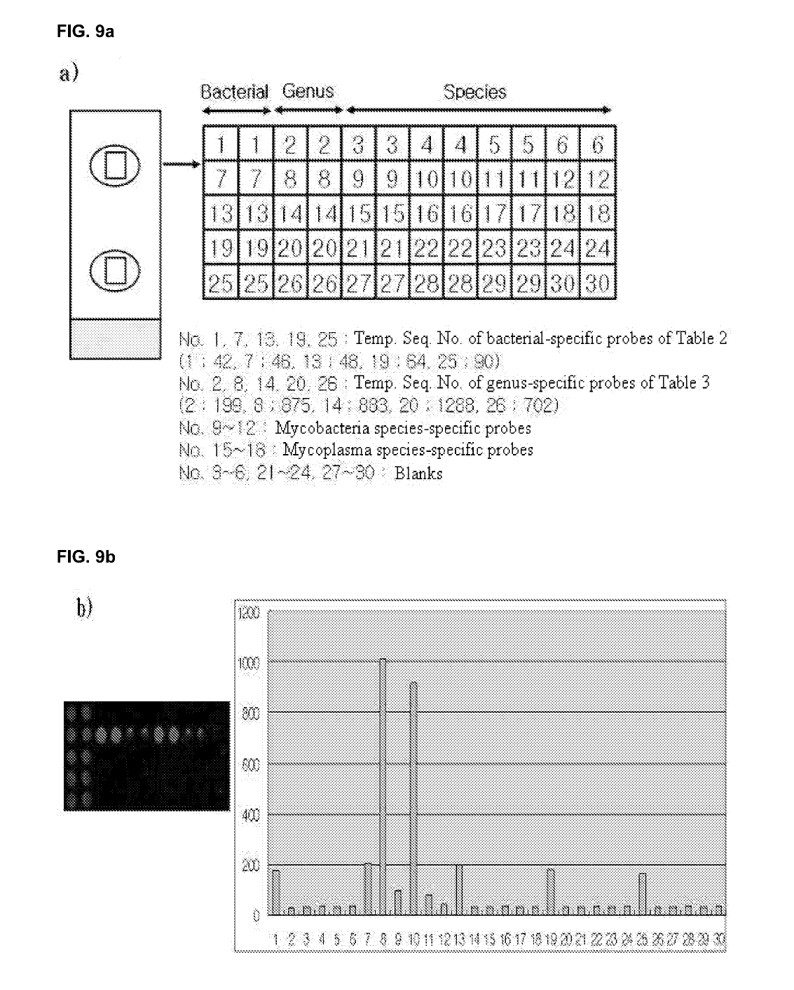

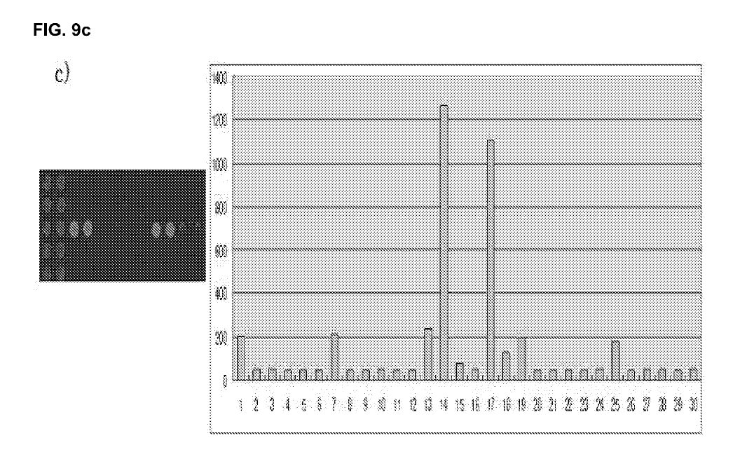

FIG. 9a depicts the microarray comprising a substrate with one set of probes to detect the presence of microorganism and to identify a bacterial genus and species together. No. 1, 7, 13, 19 and 25 are the temporary SEQ NOS of the bacterial-specific probes in Table 2 (1; 42, 7; 46, 13; 48, 19; 64, 25; 90); No. 2, 8, 14, 20 and 26, the temporary SEQ NOS of the bacterial genus-specific probes in Table 3 (2; 199, 8; 875, 14; 883, 20; 1288, 26; 702); No. 9˜12, Mycobacteria sp. specific probes; No. 15˜19, Mycoplasma sp. specific probes; and No 3˜6, 20˜24, and 27˜30 are blanks. FIG. 9b depicts the result of hybridization by using specific probes for genus Mycobacteria sp. and Mycobacterium tuberculosis (temporary SEQ NOS: 42, 46, 49, 64, 91 and 875), after performing the image analysis and estimating the intensity of its image elements. As a result, it is confirmed that the bacterial-specific probes, 1, 7, 13, 19 and 25 appear a positive signal, Mycobacterium genus-specific probe 8 (temporary SEQ NO: 875) appears a positive signal from the bacterial genus-specific probes and the bacterial species-specific probe appears a positive signal in Mycobacterium tuberculosis. FIG. 9c depicts the result of hybridization by using specific probes for Mycoplasma sp. and Mycoplasma pneumoniae (temporary SEQ NO: 42, 46, 49, 64, 91, 883) after performing the image analysis and estimating the intensity of its image elements. As a result, it is confirmed that the bacterial-specific probes, 1, 7, 13, 19 and 25 appear a positive signal, Mycobacterium genus-specific probe 14 (temporary SEQ NO: 883) appears a positive signal from the bacterial genus-specific probes and the bacterial species-specific probe appears a positive signal in Mycoplasma pneumoniae. As a consequence, the bacterial-specific and the bacterial genus and species-specific probes are reacted simultaneously to detect the presence of microorganism and identify a bacterial genus and species exactly at a time. Therefore, the present invention permits a rapid differential diagnosis to manipulate and treat diseases properly and further reduces the diagnostic cost.

The probes adopted in Examples are exemplary and can be varied in the layout of arrangement by using the novel oligonucleotides designed above.

Those skilled in the art will appreciate that the conceptions and specific embodiments disclosed in the foregoing description may be readily utilized as a basis for modifying or designing other embodiments for carrying out the same purposes of the present invention.

Those skilled in the art will also appreciate that such equivalent embodiments do not depart from the spirit and scope of the invention as set forth in the appended claims.

### INDUSTRIAL APPLICABILITY

As illustrated and confirmed above, the present invention provides the bacterial-specific and the bacterial genus and species-specific oligonucleotides designed by the target nucleotide sequences to the 23S rDNA, the PCR method using the same as a primer and the microarray using the same as a probe to detect and diagnose differentially all the microorganism such as pathogens, food-poisoning bacteria, bacteria contaminating biomedical products and environmental pollutants. In addition, the present invention provides the diagnostic kits combining the bacterial-specific and the bacterial genus and species-specific primers and probes designed by the 23S rDNA domain and the ITS region. That is to say, in the present invention, the presence of microorganism is detected by the primary screening, and if detected, microorganism is identified by the secondary screening for a differential diagnosis. Accordingly, the present invention provides the diagnostic method that is rapid and sensitive to reduce a medical cost, prevent the abuse of antibiotics and enable a proper treatment. Furthermore, several 23S rDNA genes of bacteria are newly found and determined in the nucleotide sequences to design novel oligonucleotides for a differential diagnosis. Accordingly, the present invention provides the primers and probes containing one or more target sequences that can be used to develop a very specific and sensitive method for a differential diagnosis of microorganism and the diagnostic kits comprising the same, like a PCR kit and a microarray kit.

1. A microarray comprising one or more bacterial-specific oligonucleotides for the detection of all bacteria which contain any one sequence selected among SEQ ID NO: 1 to 19 or its complementary sequence and one or more genus-specific oligonucleotides for specific detection at the bacterial genus level which contain any one sequence selected among SEQ ID NO: 20 to 189 or its complementary sequence, attached onto a substrate as probes

2. The microarray according to claim 1, which further comprises one or more species-specific oligonucleotides for the detection of the bacterial species as probes.

3. The microarray according to claim 1, wherein the probe is any one selected from a group consisting of nucleic acid analogs (or DNA minics) [deoxynucleotide (DNA), ribonucleotide (RNA), peptide nucleotide (PNA), locked nucleotide (LNA) and dihexynucleotide (HNA)].

4. The microarray according to claim 1, wherein the substrate is made of slide glass, plastic, membrane, semi-conductive chip, silicon, gel, nano material, ceramic, metallic substance, optical fiber, or their mixture.

5. A diagnostic kit for polymerase chain reaction (PCR), comprising one or more bacterial-specific oligonucleotides for the detection of all bacteria which contain any one sequence selected among SEQ ID NO: 1 to 19 or its complementary sequence and one or more genus-specific oligonucleotides for specific detection at the bacterial genus level which contain any one sequence selected among SEQ ID NO: 20 to 189 or its complementary sequence, as one set of primers.

6. The diagnostic kit for polymerase chain reaction, which further comprises one or more species-specific oligonucleotides for the detection of the bacterial species as primers.

7. A method for the detection and identification of bacteria, comprising following steps: (1) isolating nucleic acids from a specimen; (2) amplifying a target DNA within the nucleic acids by using the diagnostic kit of claim 5; and (3) analysing the amplified DNA by performing a gel electrophoresis.

8. The method for the detection and identification of bacteria according to claim 7, wherein the step (2) amplifying a target DNA is accomplished by Hot-start PCR, Nested PCR, Multiplex PCR, reverse transcriptase PCR (RT-PCR), degenerate oligonucleotide primer PCR (DOP PCR), Quantitative RT-PCR, In-Situ PCR, Micro PCR, or Lab-on a chip PCR.

9. A method for the detection and identification of bacteria, comprising following steps: (1) isolating nucleic acids from a specimen; (2) amplifying a target DNA within the nucleic acids; (3) hybridizing the amplified DNA with the probe onto the microarray of claim 1; and (4) detecting a signal generated from the DNA hybrid.

10. The method for the detection and identification of bacteria according to any one claim among claim 7 to claim 9, wherein one or more bacteria selected from a group consisting of genus Acinetobacter (SEQ ID NO: 20 to 22), genus Aeromonas (SEQ ID NO: 23 to 28), genus Bacillus (SEQ ID NO: 29 to 34), genus Bacteroides (SEQ ID NO: 35 to 41), genus Bordetella (SEQ ID NO: 42 to 44), genus Borrelia (SEQ ID NO: 45 to 47), genus Brucella (SEQ ID NO: 48 to 50), genus Burkholderia (SEQ ID NO: 51 to 53), genus Campylobacter (SEQ ID NO: 54 to 56), genus Chlamydia (SEQ ID NO: 57 to 59), genus Citrobacter (SEQ ID NO: 60 to 65), genus Clostridium (SEQ ID NO: 66 to 71), genus Corynebacterium (SEQ ID NO: 72 to 74), genus Enterbacter (SEQ ID NO: 75), genus Enterococcus (SEQ ID NO: 76 to 80), genus Fusobacterium (SEQ ID NO: 81 to 86), genus Haemophilus (SEQ ID NO: 87 to 89), genus Helicobacter (SEQ ID NO: 90 to 96), genus Klebsiella (SEQ ID NO: 97 to 102), genus Legionella (SEQ ID NO: 103 to 108), genus Listeria (SEQ ID NO: 109 to 114), genus Morganella (SEQ ID NO: 115 to 117), genus Mycobacteria (SEQ ID NO: 118 to 123), genus Mycoplasma (SEQ ID NO: 124 to 129), genus Neisseria (SEQ ID NO: 130 to 135), genus Peptococcus (SEQ ID NO: 136 to 138), genus Plesiomonas (SEQ ID NO: 139 to 141), genus Porphyromonas (SEQ ID NO: 142 to 144), genus Propionibacterium (SEQ ID NO: 145 to 147), genus Providencia (SEQ ID NO: 148 to 151), genus Pseudomonas (SEQ ID NO: 152 to 157), genus Salmonella (SEQ ID NO: 158 to 160), genus Shigella (SEQ ID NO: 161 to 164), genus Staphylococcus (SEQ ID NO: 165 to 170), genus Streptococcus (SEQ ID NO: 171 to 176), genus Treponema (SEQ ID NO: 177 to 179), genus Ureaplasma (SEQ ID NO: 180 to 182), genus Vibrio (SEQ ID NO: 183 to 185), and genus Yersinia (SEQ ID NO: 186 to 189), can be diagnosed simultaneously.

11. A bacterial-specific oligonucleotide for the detection of all bacteria, which contains any one sequence selected among SEQ ID NO: 1 to 19 or its complementary sequence.

12. A genus-specific oligonucleotide for specific detection at the bacterial genus level, which contains any one sequence selected among SEQ ID NO: 20 to 189 or its complementary sequence.


SEQUENCE LISTING (189 sequences)

SEQ ID NO: 1 - DNA, 20 bp, Artificial Sequence
  Bacterial specific primer/probe
tanggcgggacacgtgaaat

SEQ ID NO: 2 - DNA, 19 bp, Artificial Sequence
  Bacterial specific primer/probe
ccgatagtgaaccagtacc

SEQ ID NO: 3 - DNA, 20 bp, Artificial Sequence
  Bacterial specific primer/probe
ccagtaccgtgagggaaagg

SEQ ID NO: 4 - DNA, 20 bp, Artificial Sequence
  Bacterial specific primer/probe
nagaacctgaaaccgtgtgc

SEQ ID NO: 5 - DNA, 20 bp, Artificial Sequence
  Bacterial specific primer/probe
gggaaacaacccagaccgcc

SEQ ID NO: 6 - DNA, 21 bp, Artificial Sequence
  Bacterial specific primer/probe
gttggcttagaagcagccatc

SEQ ID NO: 7 - DNA, 23 bp, Artificial Sequence
  Bacterial specific primer/probe
gagagaactcgggtgaaggaact

SEQ ID NO: 8 - DNA, 19 bp, Artificial Sequence
  Bacterial specific primer/probe
ccgtaacttcgggagaagg

SEQ ID NO: 9 - DNA, 17 bp, Artificial Sequence
  Bacterial specific primer/probe
ccvgtaaacggcggccg

SEQ ID NO: 10 - DNA, 24 bp, Artificial Sequence
  Bacterial specific primer/probe
cggcggccgtaactataacggtcc

SEQ ID NO: 11 - DNA, 22 bp, Artificial Sequence
  Bacterial specific primer/probe
ctataacggtcctaaggtagcg

SEQ ID NO: 12 - DNA, 21 bp, Artificial Sequence
  Bacterial specific primer/probe
ctaaggtagcgaaattccttg

SEQ ID NO: 13 - DNA, 21 bp, Artificial Sequence
  Bacterial specific primer/probe
gtcgggtaagttccgacctgc

SEQ ID NO: 14 - DNA, 21 bp, Artificial Sequence
  Bacterial specific primer/probe
gacggaaagaccccgtgaacc

SEQ ID NO: 15 - DNA, 21 bp, Artificial Sequence
  Bacterial specific primer/probe
tgggtagtttgactggggcgg

SEQ ID NO: 16 - DNA, 19 bp, Artificial Sequence
  Bacterial specific primer/probe
tagtgatccggtggttctg

SEQ ID NO: 17 - DNA, 20 bp, Artificial Sequence
  Bacterial specific primer/probe
ccatcgctcaacggataaaa

SEQ ID NO: 18 - DNA, 19 bp, Artificial Sequence
  Bacterial specific primer/probe
gtttggcacctcgatgtcg

SEQ ID NO: 19 - DNA, 20 bp, Artificial Sequence
  Bacterial specific primer/probe
gtcgtgagacagttcggtcc

SEQ ID NO: 20 - DNA, 22 bp, Artificial Sequence
  Genus-specific primer/probe
acccacctactttaaggtaggt

SEQ ID NO: 21 - DNA, 22 bp, Artificial Sequence
  Genus-specific primer/probe
aaaactatgtgccgaagctgcg

SEQ ID NO: 22 - DNA, 23 bp, Artificial Sequence
  Genus-specific primer/probe
tggtcaggtcgagtagaccaagg

SEQ ID NO: 23 - DNA, 20 bp, Artificial Sequence
  Genus-specific primer/probe
agtggaayggtcctggaaag

SEQ ID NO: 24 - DNA, 21 bp, Artificial Sequence
  Genus-specific primer/probe
ctgggcgtctagttgcaaggt

SEQ ID NO: 25 - DNA, 20 bp, Artificial Sequence
  Genus-specific primer/probe
ggttaatattcctgcacgac

SEQ ID NO: 26 - DNA, 20 bp, Artificial Sequence
  Genus-specific primer/probe
ctgcacgacttgtaattgcg

SEQ ID NO: 27 - DNA, 27 bp, Artificial Sequence
  Genus-specific primer/probe
cgtgggcgttggatgattgaagggagt

SEQ ID NO: 28 - DNA, 21 bp, Artificial Sequence
  Genus-specific primer/probe
cactgcccggtagctaagttc

SEQ ID NO: 29 - DNA, 22 bp, Artificial Sequence
  Genus-specific primer/probe
gaggaagagaaagcaaatgcga

SEQ ID NO: 30 - DNA, 27 bp, Artificial Sequence
  Genus-specific primer/probe
tcaggtaacactgaatggaggcccgaa

SEQ ID NO: 31 - DNA, 20 bp, Artificial Sequence
  Genus-specific primer/probe
aagagtcttggaggtagagc

SEQ ID NO: 32 - DNA, 20 bp, Artificial Sequence
  Genus-specific primer/probe
ccgaattcagtcaaactccg

SEQ ID NO: 33 - DNA, 21 bp, Artificial Sequence
  Genus-specific primer/probe
gactctgcgccgaaaatgtac

SEQ ID NO: 34 - DNA, 20 bp, Artificial Sequence
  Genus-specific primer/probe
agcgctagcttcggtggagg

SEQ ID NO: 35 - DNA, 20 bp, Artificial Sequence
  Genus-specific primer/probe
catccatctttgatggaagc

SEQ ID NO: 36 - DNA, 20 bp, Artificial Sequence
  Genus-specific primer/probe
gtcgcaagacatttgatgag

SEQ ID NO: 37 - DNA, 20 bp, Artificial Sequence
  Genus-specific primer/probe
gaagagtgaaatagtccctg

SEQ ID NO: 38 - DNA, 26 bp, Artificial Sequence
  Genus-specific primer/probe
agttctatcacaggagtgagggcatg

SEQ ID NO: 39 - DNA, 22 bp, Artificial Sequence
  Genus-specific primer/probe
cagattgctaagacagctagga

SEQ ID NO: 40 - DNA, 25 bp, Artificial Sequence
  Genus-specific primer/probe
ctggagcgtgtggaaaagcaaatgt

SEQ ID NO: 41 - DNA, 24 bp, Artificial Sequence
  Genus-specific primer/probe
gacacacgtagtcgggttgaatat

SEQ ID NO: 42 - DNA, 21 bp, Artificial Sequence
  Genus-specific primer/probe
gcattttgcatagtcgaacgg

SEQ ID NO: 43 - DNA, 21 bp, Artificial Sequence
  Genus-specific primer/probe
aaatccgggcgcgagaatcaa

SEQ ID NO: 44 - DNA, 24 bp, Artificial Sequence
  Genus-specific primer/probe
cagaactcgagtcgccagattcga

SEQ ID NO: 45 - DNA, 20 bp, Artificial Sequence
  Genus-specific primer/probe
ttcgtccatctcggtcctct

SEQ ID NO: 46 - DNA, 22 bp, Artificial Sequence
  Genus-specific primer/probe
gcgaatgggaatctcacccatt

SEQ ID NO: 47 - DNA, 27 bp, Artificial Sequence
  Genus-specific primer/probe
actgagtttggtaccctttgacaggcc

SEQ ID NO: 48 - DNA, 27 bp, Artificial Sequence
  Genus-specific primer/probe
tctgttttgttggagcaacgctggatg

SEQ ID NO: 49 - DNA, 27 bp, Artificial Sequence
  Genus-specific primer/probe
ggggagtactaatcggcagacacacgg

SEQ ID NO: 50 - DNA, 29 bp, Artificial Sequence
  Genus-specific primer/probe
cgtgacctcactatgggcaaccatagggg

SEQ ID NO: 51 - DNA, 20 bp, Artificial Sequence
  Genus-specific primer/probe
tgcggccatagcaggtgata

SEQ ID NO: 52 - DNA, 24 bp, Artificial Sequence
  Genus-specific primer/probe
aggtccgaacccactaacgttgaa

SEQ ID NO: 53 - DNA, 27 bp, Artificial Sequence
  Genus-specific primer/probe
gaggagtacgaaggtacgctaggtacg

SEQ ID NO: 54 - DNA, 25 bp, Artificial Sequence
  Genus-specific primer/probe
tcgaccacgatccaaccctaaatac

SEQ ID NO: 55 - DNA, 21 bp, Artificial Sequence
  Genus-specific primer/probe
tgtgagaatcattaacgccgt

SEQ ID NO: 56 - DNA, 27 bp, Artificial Sequence
  Genus-specific primer/probe
agagatttgaccctagtacgagaggac

SEQ ID NO: 57 - DNA, 20 bp, Artificial Sequence
  Genus-specific primer/probe
ataagcaaagacccggaggt

SEQ ID NO: 58 - DNA, 24 bp, Artificial Sequence
  Genus-specific primer/probe
tagggttagcctcggataataagc

SEQ ID NO: 59 - DNA, 29 bp, Artificial Sequence
  Genus-specific primer/probe
aaggtatcccaatgagactccatgtagac

SEQ ID NO: 60 - DNA, 24 bp, Artificial Sequence
  Genus-specific primer/probe
gtcacagttaagtgggaaacgatg

SEQ ID NO: 61 - DNA, 30 bp, Artificial Sequence
  Genus-specific primer/probe
cggaagatgtaacggggctaaactgtgcac

SEQ ID NO: 62 - DNA, 25 bp, Artificial Sequence
  Genus-specific primer/probe
gggctaaactgtgcaccgaagctgc

SEQ ID NO: 63 - DNA, 25 bp, Artificial Sequence
  Genus-specific primer/probe
atccggtacctttttaacgctgagg

SEQ ID NO: 64 - DNA, 21 bp, Artificial Sequence
  Genus-specific primer/probe
tgatggggttatcgtaagaga

SEQ ID NO: 65 - DNA, 24 bp, Artificial Sequence
  Genus-specific primer/probe
gtgttctaacgtggacccgttacc

SEQ ID NO: 66 - DNA, 20 bp, Artificial Sequence
  Genus-specific primer/probe
cggtatgtagcaaggttaag

SEQ ID NO: 67 - DNA, 30 bp, Artificial Sequence
  Genus-specific primer/probe
gatgagctgtggatagcggagaaattccaa

SEQ ID NO: 68 - DNA, 27 bp, Artificial Sequence
  Genus-specific primer/probe
agctcactagtcaagagatcctgcgcc

SEQ ID NO: 69 - DNA, 22 bp, Artificial Sequence
  Genus-specific primer/probe
acccgcacgaatggcgtaatga

SEQ ID NO: 70 - DNA, 22 bp, Artificial Sequence
  Genus-specific primer/probe
attggacggaaagaccccgtag

SEQ ID NO: 71 - DNA, 22 bp, Artificial Sequence
  Genus-specific primer/probe
ttcggtattgtctgtacaggat

SEQ ID NO: 72 - DNA, 22 bp, Artificial Sequence
  Genus-specific primer/probe
tgtggagtgtgtgcgagtgaga

SEQ ID NO: 73 - DNA, 22 bp, Artificial Sequence
  Genus-specific primer/probe
cttggcccatgatctgggttgg

SEQ ID NO: 74 - DNA, 26 bp, Artificial Sequence
  Genus-specific primer/probe
gactagtgatccggcacctacttgtg

SEQ ID NO: 75 - DNA, 20 bp, Artificial Sequence
  Genus-specific primer/probe
tgcgggtggagctgaaatca

SEQ ID NO: 76 - DNA, 20 bp, Artificial Sequence
  Genus-specific primer/probe
ttggggttgtaggactccra

SEQ ID NO: 77 - DNA, 22 bp, Artificial Sequence
  Genus-specific primer/probe
cacctaggaggatcctgagtac

SEQ ID NO: 78 - DNA, 21 bp, Artificial Sequence
  Genus-specific primer/probe
agaatgatggaggtagagcac

SEQ ID NO: 79 - DNA, 21 bp, Artificial Sequence
  Genus-specific primer/probe
gttatgvccactctaacccgc

SEQ ID NO: 80 - DNA, 22 bp, Artificial Sequence
  Genus-specific primer/probe
gtaagatccctgagagatgatc

SEQ ID NO: 81 - DNA, 20 bp, Artificial Sequence
  Genus-specific primer/probe
gtaacgcgaggaaaagaaag

SEQ ID NO: 82 - DNA, 27 bp, Artificial Sequence
  Genus-specific primer/probe
ctcccaagtaacatggaacacgaggaa

SEQ ID NO: 83 - DNA, 21 bp, Artificial Sequence
  Genus-specific primer/probe
ctgagtaagccgggacctaag

SEQ ID NO: 84 - DNA, 20 bp, Artificial Sequence
  Genus-specific primer/probe
acgggaggcctggtgaaatt

SEQ ID NO: 85 - DNA, 20 bp, Artificial Sequence
  Genus-specific primer/probe
gcatgtataggatagttggg

SEQ ID NO: 86 - DNA, 29 bp, Artificial Sequence
  Genus-specific primer/probe
cctggggataacaggctgatcctacccga

SEQ ID NO: 87 - DNA, 20 bp, Artificial Sequence
  Genus-specific primer/probe
tgaaccggtcgaagatacca

SEQ ID NO: 88 - DNA, 30 bp, Artificial Sequence
  Genus-specific primer/probe
aggagcacgaaggtttgctaatcacggtcg

SEQ ID NO: 89 - DNA, 23 bp, Artificial Sequence
  Genus-specific primer/probe
gtttgctaatcacggtcggacat

SEQ ID NO: 90 - DNA, 17 bp, Artificial Sequence
  Genus-specific primer/probe
ggggttgaggactgcaa

SEQ ID NO: 91 - DNA, 22 bp, Artificial Sequence
  Genus-specific primer/probe
aaccgcagtgagcggagtgaaa

SEQ ID NO: 92 - DNA, 23 bp, Artificial Sequence
  Genus-specific primer/probe
gagccctatgatttatcagggtg

SEQ ID NO: 93 - DNA, 23 bp, Artificial Sequence
  Genus-specific primer/probe
ctcgtacccattgaaacgggttg

SEQ ID NO: 94 - DNA, 20 bp, Artificial Sequence
  Genus-specific primer/probe
aggtatagcctcaagtgata

SEQ ID NO: 95 - DNA, 22 bp, Artificial Sequence
  Genus-specific primer/probe
cgtatcttgggagtcaggcggt

SEQ ID NO: 96 - DNA, 22 bp, Artificial Sequence
  Genus-specific primer/probe
ggtctcagcaaagagtccctcc

SEQ ID NO: 97 - DNA, 20 bp, Artificial Sequence
  Genus-specific primer/probe
cgacacacagctaatgtgtg

SEQ ID NO: 98 - DNA, 20 bp, Artificial Sequence
  Genus-specific primer/probe
caaattttcgcgacacgacg

SEQ ID NO: 99 - DNA, 27 bp, Artificial Sequence
  Genus-specific primer/probe
attttcgcgacacgacgatgttttacg

SEQ ID NO: 100 - DNA, 20 bp, Artificial Sequence
  Genus-specific primer/probe
gtgaagtgacttgctcatgg

SEQ ID NO: 101 - DNA, 25 bp, Artificial Sequence
  Genus-specific primer/probe
gaagtgacttgctcatggagctgaa

SEQ ID NO: 102 - DNA, 20 bp, Artificial Sequence
  Genus-specific primer/probe
gctcatggagctgaaatcag

SEQ ID NO: 103 - DNA, 25 bp, Artificial Sequence
  Genus-specific primer/probe
aggtttgatgaggaactaggcacgc

SEQ ID NO: 104 - DNA, 26 bp, Artificial Sequence
  Genus-specific primer/probe
taccggctaattgaatcacgggagac

SEQ ID NO: 105 - DNA, 28 bp, Artificial Sequence
  Genus-specific primer/probe
gtacttgcatgtaggggggaagacttgg

SEQ ID NO: 106 - DNA, 22 bp, Artificial Sequence
  Genus-specific primer/probe
ttactttcagagctggagaggg

SEQ ID NO: 107 - DNA, 28 bp, Artificial Sequence
  Genus-specific primer/probe
ttgaggttctaacttggtccagtaatcc

SEQ ID NO: 108 - DNA, 25 bp, Artificial Sequence
  Genus-specific primer/probe
ggtcggacatcgtaccaagagtgta

SEQ ID NO: 109 - DNA, 22 bp, Artificial Sequence
  Genus-specific primer/probe
ctttccctccagagtggatcct

SEQ ID NO: 110 - DNA, 22 bp, Artificial Sequence
  Genus-specific primer/probe
gctagcctcgaggtaaagagtc

SEQ ID NO: 111 - DNA, 21 bp, Artificial Sequence
  Genus-specific primer/probe
gtgcgtccaagcagtgagtgt

SEQ ID NO: 112 - DNA, 21 bp, Artificial Sequence
  Genus-specific primer/probe
cgaagcatgagctgtgatggg

SEQ ID NO: 113 - DNA, 23 bp, Artificial Sequence
  Genus-specific primer/probe
tggaatgtttgtaccgcttgtac

SEQ ID NO: 114 - DNA, 22 bp, Artificial Sequence
  Genus-specific primer/probe
agcgcgtggggagacagtgtca

SEQ ID NO: 115 - DNA, 20 bp, Artificial Sequence
  Genus-specific primer/probe
agcacagtgagcggagcata

SEQ ID NO: 116 - DNA, 20 bp, Artificial Sequence
  Genus-specific primer/probe
gtgttaagagaacagtctgg

SEQ ID NO: 117 - DNA, 27 bp, Artificial Sequence
  Genus-specific primer/probe
atctgaaagcactggtgttgtgagtcc

SEQ ID NO: 118 - DNA, 30 bp, Artificial Sequence
  Genus-specific primer/probe
gccagggtgaagcgcgggtaagaccgcgtg

SEQ ID NO: 119 - DNA, 20 bp, Artificial Sequence
  Genus-specific primer/probe
tagctcactggtcaagtgat

SEQ ID NO: 120 - DNA, 21 bp, Artificial Sequence
  Genus-specific primer/probe
tccgtgcgaagtcgcaagacg

SEQ ID NO: 121 - DNA, 22 bp, Artificial Sequence
  Genus-specific primer/probe
gtctcaaccatagactcggcga

SEQ ID NO: 122 - DNA, 20 bp, Artificial Sequence
  Genus-specific primer/probe
aaggttccctcaacctggwc

SEQ ID NO: 123 - DNA, 20 bp, Artificial Sequence
  Genus-specific primer/probe
agcatctaagcgggaaacct

SEQ ID NO: 124 - DNA, 21 bp, Artificial Sequence
  Genus-specific primer/probe
tgtgtagtggcgagcgaaagc

SEQ ID NO: 125 - DNA, 21 bp, Artificial Sequence
  Genus-specific primer/probe
gaatctgcccagaccattggg

SEQ ID NO: 126 - DNA, 22 bp, Artificial Sequence
  Genus-specific primer/probe
catatgcctacaacgtgtcaga

SEQ ID NO: 127 - DNA, 22 bp, Artificial Sequence
  Genus-specific primer/probe
acagctcacttgtcgagtgttt

SEQ ID NO: 128 - DNA, 17 bp, Artificial Sequence
  Genus-specific primer/probe
ggctaagtatattaccg

SEQ ID NO: 129 - DNA, 20 bp, Artificial Sequence
  Genus-specific primer/probe
tgatgtatatggggtgacac

SEQ ID NO: 130 - DNA, 28 bp, Artificial Sequence
  Genus-specific primer/probe
ggcaataaagcaatgatcccgcgatgtc

SEQ ID NO: 131 - DNA, 20 bp, Artificial Sequence
  Genus-specific primer/probe
gaaacagaacctgaaacctg

SEQ ID NO: 132 - DNA, 20 bp, Artificial Sequence
  Genus-specific primer/probe
gctaaggtcccaaatgatag

SEQ ID NO: 133 - DNA, 29 bp, Artificial Sequence
  Genus-specific primer/probe
ttgaagatgtgagagcatcggatcgaagc

SEQ ID NO: 134 - DNA, 25 bp, Artificial Sequence
  Genus-specific primer/probe
gcattggactttgaagtcacttgtg

SEQ ID NO: 135 - DNA, 22 bp, Artificial Sequence
  Genus-specific primer/probe
gttctaacccagacccgtcatc

SEQ ID NO: 136 - DNA, 30 bp, Artificial Sequence
  Genus-specific primer/probe
gcaagcagaccttgacacgtagatatccga

SEQ ID NO: 137 - DNA, 22 bp, Artificial Sequence
  Genus-specific primer/probe
aatagggcgcaagtttcttggc

SEQ ID NO: 138 - DNA, 22 bp, Artificial Sequence
  Genus-specific primer/probe
gttggacacaccgctggttaac

SEQ ID NO: 139 - DNA, 28 bp, Artificial Sequence
  Genus-specific primer/probe
ggacggaaaagggttggtgagctgggat

SEQ ID NO: 140 - DNA, 25 bp, Artificial Sequence
  Genus-specific primer/probe
ttaggtgttattgggctaggggagc

SEQ ID NO: 141 - DNA, 22 bp, Artificial Sequence
  Genus-specific primer/probe
gttgtcccgtaatccgggatac

SEQ ID NO: 142 - DNA, 23 bp, Artificial Sequence
  Genus-specific primer/probe
cgggaatcggcacatacgaattg

SEQ ID NO: 143 - DNA, 28 bp, Artificial Sequence
  Genus-specific primer/probe
ttggcatggataatacacgggcataagc

SEQ ID NO: 144 - DNA, 29 bp, Artificial Sequence
  Genus-specific primer/probe
gccagtattcgtggagtcgatgttgaaat

SEQ ID NO: 145 - DNA, 27 bp, Artificial Sequence
  Genus-specific primer/probe
actgtctccaccatgaactcggtgaaa

SEQ ID NO: 146 - DNA, 24 bp, Artificial Sequence
  Genus-specific primer/probe
ataccactctggtcgttctggtta

SEQ ID NO: 147 - DNA, 20 bp, Artificial Sequence
  Genus-specific primer/probe
ctctggtcgttctggttatc

SEQ ID NO: 148 - DNA, 22 bp, Artificial Sequence
  Genus-specific primer/probe
tcggtcattcaaacgagtggca

SEQ ID NO: 149 - DNA, 24 bp, Artificial Sequence
  Genus-specific primer/probe
cccaacgcagaagttcaccacgca

SEQ ID NO: 150 - DNA, 26 bp, Artificial Sequence
  Genus-specific primer/probe
cacagccatgacagtcaggtgatcgt

SEQ ID NO: 151 - DNA, 21 bp, Artificial Sequence
  Genus-specific primer/probe
gggagccttgatttatcaggg

SEQ ID NO: 152 - DNA, 20 bp, Artificial Sequence
  Genus-specific primer/probe
atgcagtgtatccgcggcta

SEQ ID NO: 153 - DNA, 20 bp, Artificial Sequence
  Genus-specific primer/probe
tatccgcggctagacggaaa

SEQ ID NO: 154 - DNA, 23 bp, Artificial Sequence
  Genus-specific primer/probe
taaaggcaaaagcgcgcttgact

SEQ ID NO: 155 - DNA, 28 bp, Artificial Sequence
  Genus-specific primer/probe
cctatctgccgtggacgtttgagatttg

SEQ ID NO: 156 - DNA, 23 bp, Artificial Sequence
  Genus-specific primer/probe
ttgagatttgagaggggctgctc

SEQ ID NO: 157 - DNA, 22 bp, Artificial Sequence
  Genus-specific primer/probe
attgcccgtgaggcttgaccat

SEQ ID NO: 158 - DNA, 21 bp, Artificial Sequence
  Genus-specific primer/probe
gcatgtgtgttagtggaagcg

SEQ ID NO: 159 - DNA, 21 bp, Artificial Sequence
  Genus-specific primer/probe
cacgctgacacgtaggtgaag

SEQ ID NO: 160 - DNA, 20 bp, Artificial Sequence
  Genus-specific primer/probe
agaaggcacgctgacacgta

SEQ ID NO: 161 - DNA, 20 bp, Artificial Sequence
  Genus-specific primer/probe
aagcagtgggagcacgctta

SEQ ID NO: 162 - DNA, 21 bp, Artificial Sequence
  Genus-specific primer/probe
cttaggcgtgtgactgcgtac

SEQ ID NO: 163 - DNA, 20 bp, Artificial Sequence
  Genus-specific primer/probe
tgtttcggcaagggggtcat

SEQ ID NO: 164 - DNA, 23 bp, Artificial Sequence
  Genus-specific primer/probe
gaaaatcaaggccgaggcgtgat

SEQ ID NO: 165 - DNA, 23 bp, Artificial Sequence
  Genus-specific primer/probe
gattaagttattaagggcgcacg

SEQ ID NO: 166 - DNA, 20 bp, Artificial Sequence
  Genus-specific primer/probe
agtatttggtcgtagacccg

SEQ ID NO: 167 - DNA, 20 bp, Artificial Sequence
  Genus-specific primer/probe
tagtcgagtgacactgcgcc

SEQ ID NO: 168 - DNA, 23 bp, Artificial Sequence
  Genus-specific primer/probe
gagaaggggtgctctttagggtt

SEQ ID NO: 169 - DNA, 22 bp, Artificial Sequence
  Genus-specific primer/probe
tggttagcttctgcgaagctac

SEQ ID NO: 170 - DNA, 23 bp, Artificial Sequence
  Genus-specific primer/probe
cccaacttcggttataagatccc

SEQ ID NO: 171 - DNA, 22 bp, Artificial Sequence
  Genus-specific primer/probe
cagggcacgttgaaaagtgctt

SEQ ID NO: 172 - DNA, 20 bp, Artificial Sequence
  Genus-specific primer/probe
acaggtagtcgaggcgagta

SEQ ID NO: 173 - DNA, 20 bp, Artificial Sequence
  Genus-specific primer/probe
cccttgtgttatggctactc

SEQ ID NO: 174 - DNA, 26 bp, Artificial Sequence
  Genus-specific primer/probe
acagtgtctgacgggcagtttgactg

SEQ ID NO: 175 - DNA, 20 bp, Artificial Sequence
  Genus-specific primer/probe
ggaaatttgagaggatctgc

SEQ ID NO: 176 - DNA, 20 bp, Artificial Sequence
  Genus-specific primer/probe
atcagtaagagccctgagag

SEQ ID NO: 177 - DNA, 28 bp, Artificial Sequence
  Genus-specific primer/probe
aataatccggcctatagcagaaaggttt

SEQ ID NO: 178 - DNA, 27 bp, Artificial Sequence
  Genus-specific primer/probe
ggtaggggagcattccatgtactgatg

SEQ ID NO: 179 - DNA, 30 bp, Artificial Sequence
  Genus-specific primer/probe
acccatagttagacggaaagaccccgtgaa

SEQ ID NO: 180 - DNA, 30 bp, Artificial Sequence
  Genus-specific primer/probe
aggccaaaccgaatttcgattcggggttgt

SEQ ID NO: 181 - DNA, 26 bp, Artificial Sequence
  Genus-specific primer/probe
gggtgctcgctgtaaaaggtgagccg

SEQ ID NO: 182 - DNA, 26 bp, Artificial Sequence
  Genus-specific primer/probe
tggagtcaacgttggaatactaccct

SEQ ID NO: 183 - DNA, 21 bp, Artificial Sequence
  Genus-specific primer/probe
tagtgcaatggcataagcccg

SEQ ID NO: 184 - DNA, 20 bp, Artificial Sequence
  Genus-specific primer/probe
cacagcactgtgcaaaatcg

SEQ ID NO: 185 - DNA, 26 bp, Artificial Sequence
  Genus-specific primer/probe
gtcattgatgccatgcttccaggaaa

SEQ ID NO: 186 - DNA, 20 bp, Artificial Sequence
  Genus-specific primer/probe
tcgcacggtacagggtgata

SEQ ID NO: 187 - DNA, 22 bp, Artificial Sequence
  Genus-specific primer/probe
gcacggtacagggtgatagtcc

SEQ ID NO: 188 - DNA, 20 bp, Artificial Sequence
  Genus-specific primer/probe
ggtaaatccggttgcttatc

SEQ ID NO: 189 - DNA, 23 bp, Artificial Sequence
  Genus-specific primer/probe
gtttgatgttctaactcggcccc